(19) United States
(12) Patent Application Publication
(10) Pub. No.: US 2021/0077469 A1
(43) Pub. Date: Mar. 18, 2021

(54) PHARMACEUTICAL COMBINATIONS OF EGFR INHIBITORS AND METHODS OF USE THEREOF

(71) Applicant: DANA-FARBER CANCER INSTITUTE, INC., BOSTON, MA (US)

(72) Inventors: NATHANAEL S. GRAY, JAMAICA PLAIN, MA (US); DRIES DE CLERCQ, BOSTON, MA (US); JAEBONG JANG, BOSTON, MA (US); PASI JANNE, NEEDHAM, MA (US); CIRIC TO, BOSTON, MA (US); MICHAEL ECK, BOSTON, MA (US); EUNYOUNG PARK, BOSTON, MA (US); DAVID HEPPNER, BOSTON, MA (US)

(21) Appl. No.: 16/971,021

(22) PCT Filed: Feb. 20, 2019
(86) PCT No.: PCT/US19/18770

§ 371 Date: Aug. 19, 2020

Related U.S. Application Data
(60) Provisional application No. 62/632,798, filed on Feb. 20, 2018.

(51) Int. Cl. A61K 31/427; A61K 31/496; A61K 31/497

(52) U.S. Cl. CPC A61K 31/427; A61K 31/497; A61K 31/496

(57) ABSTRACT

The application relates to a pharmaceutical combination of an allosteric EGFR inhibitor of Formula I: (I), or a pharmaceutically acceptable salt, hydrate, or solvate thereof, and an ATP-competitive EGFR inhibitor of Formula I′: (I′), or a pharmaceutically acceptable salt, hydrate, or solvate thereof, which modulates the activity of EGFR, a pharmaceutical composition comprising the combination, and a method of treating or preventing a disease in which EGFR plays a role.

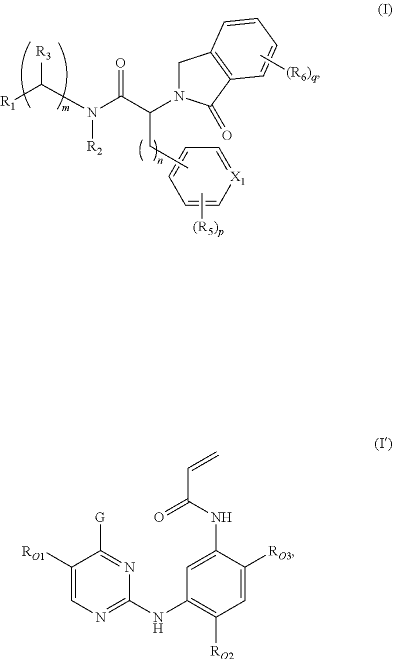

### CROSS-REFERENCE TO RELATED APPLICATION

This application claims priority to, and the benefit of, U.S. Provisional Application No. 62/632,798, filed on Feb. 20, 2018, the entire contents of which are incorporated herein by reference.

### GOVERNMENT SUPPORT

The work described herein was supported by the National Institutes of Health, NIH Grant Nos. R01 CA201049 and P01 CA154303. The U.S. Government has certain rights to the claimed invention.

### BACKGROUND

The epidermal growth factor receptor (EGFR, Erb-B1) belongs to a family of proteins involved in cell proliferation. EGFR overexpression is present in at least 70% of human cancers, such as non-small cell lung carcinoma (NSCLC), breast cancer, glioma, and prostate cancer. The EGFR-TK is therefore widely recognized as a target for the design and development of therapies that can specifically bind and inhibit tyrosine kinase activity and its signal transduction pathway in cancer cells, and thus can serve as diagnostic or therapeutic agents.

EGFR tyrosine kinase inhibitors (TKIs) are effective clinical therapies for EGFR mutant advanced non-small cell lung cancer (NSCLC) patients. However, the vast majority of patients develop disease progression following successful treatment with an EGFR TKI. The most common mechanism of acquired resistance is a secondary mutation T790M, which leads to an increase in ATP affinity, thus making it more difficult for reversible EGFR TKIs gefitinib and erlotinib to bind the EGFR TKI domain. Covalent EGFR inhibitors have emerged as strategies to inhibit EGFR T790M containing cancers. Afatinib is a potent inhibitor of both mutant and wild type (WT) EGFR, but is only effective in EGFR TKI naive EGFR mutant cancers, has a RR of 10% in patients with NSCLC resistant to gefitinib or erotinib, and suffers from toxicities from inhibition of WT EGFR. Other irreversible EGFR inhibitors, such as WZ4002, CO-1686, and AZD9291, overcome many of the limitations of afatinib. They are not only more potent on EGFR T790M, but also selectively inhibit mutant over WT EGFR.

However, all current EGFR TKIs target the ATP binding site, and are rendered impotent by the C797S mutation arising in treated patients. Cetuximab, an anti-EGFR antibody that blocks receptor dimerization is not effective in EGFR-mutant NSCLC, because mutational activation of the kinase is effectively “downstream” of receptor dimerization. Hence, alternative strategies to inhibit EGFR are needed. The present application addresses the need.

### SUMMARY

The present application relates to a pharmaceutical combination comprising an allosteric EGFR inhibitor and an ATP-competitive EGFR inhibitor, which is capable of inhibiting drug resistant forms of EGFR. The application features methods of treating or preventing a disease in which EGFR plays a role in a subject in need thereof by administering to the subject a therapeutically effective amount of an allosteric EGFR inhibitor in combination with (e.g., in temporal proximity with) a therapeutically effective amount of an ATP-competitive EGFR inhibitor. The methods of the application can be used to treat or prevent diseases in which EGFR plays a role by inhibiting the kinase activity of EGFR.

A first aspect of the application relates to a pharmaceutical combination comprising an allosteric EGFR inhibitor and an ATP-competitive EGFR inhibitor.

In one embodiment, the allosteric EGFR inhibitor is a compound of Formula I:

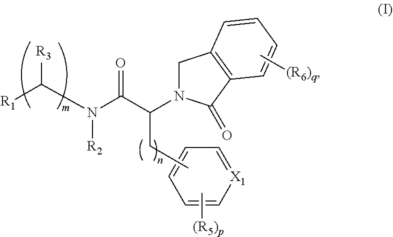

or a pharmaceutically acceptable salt, hydrate, or solvate thereof, wherein each of the variables in Formula I is described herein in detail below.

In one embodiment, the ATP-competitive EGFR inhibitor is a compound of Formula I′:

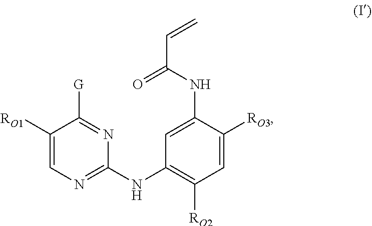

or a pharmaceutically acceptable salt, hydrate, or solvate thereof, wherein each of the variables in Formula I′ is described herein in detail below.

In one embodiment, the allosteric EGFR inhibitor is Compound A:

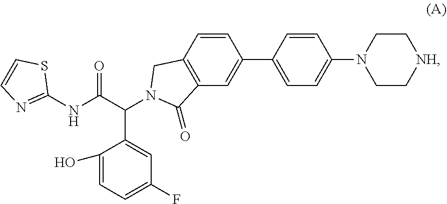 [SMILES: O=C(Nc1nccs1)C(c1cc(F)ccc1O)N1Cc2ccc(-c3ccc(N4CCNCC4)cc3)cc2C1=O]

or a pharmaceutically acceptable salt, hydrate, or solvate thereof, and the ATP-competitive EGFR inhibitor is Compound O:

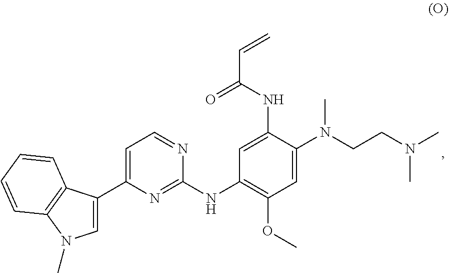 [SMILES: C=CC(=O)Nc1cc(Nc2nccc(-c3cn(C)c4ccccc34)n2)c(OC)cc1N(C)CCN(C)C]

or a pharmaceutically acceptable salt, hydrate, or solvate thereof.

In one embodiment, Compound A is of the following structure:

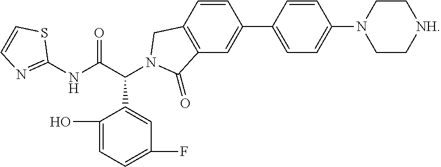 [SMILES: O=C(Nc1nccs1)[C@@H](c1cc(F)ccc1O)N1Cc2ccc(-c3ccc(N4CCNCC4)cc3)cc2C1=O]

Another aspect of the application relates to a pharmaceutical composition comprising a pharmaceutical combination of the application, and a pharmaceutically acceptable carrier.

Another aspect of the application relates to a kit comprising an allosteric EGFR inhibitor, as described herein, and an ATP-competitive EGFR inhibitor, as described herein.

Another aspect of the application relates to a kit comprising a pharmaceutical combination of the application.

Another aspect of the present application relates to a method of inhibiting a kinase (e.g., EGFR). The method comprises administering to a subject in need thereof an effective amount of a pharmaceutical combination of the application, or an effective amount of an allosteric EGFR inhibitor, as described herein, in combination with (e.g., in temporal proximity with) an effective amount of an ATP-competitive EGFR inhibitor, as described herein.

Another aspect of the present application relates to a method of treating or preventing a disease (e.g., a disease in which EGFR plays a role). The method comprises administering to a subject in need thereof an effective amount of a pharmaceutical combination of the application, or an effective amount of an allosteric EGFR inhibitor, as described herein, in combination with (e.g., in temporal proximity with) an effective amount of an ATP-competitive EGFR inhibitor, as described herein.

Another aspect of the present application relates to a method of treating or preventing a disease resistant to an EGFR targeted therapy, such as a therapy with gefitinib, erlotinib, afatinib, AZD9291, CO-1686, or WZ4002. The method comprises administering to a subject in need thereof an effective amount of a pharmaceutical combination of the application, or an effective amount of an allosteric EGFR inhibitor, as described herein, in combination with (e.g., in temporal proximity with) an effective amount of an ATP-competitive EGFR inhibitor, as described herein.

Another aspect of the present application relates to a method of treating or preventing cancer, wherein the cell of the cancer comprises an activated EGFR. The method comprises administering to a subject in need thereof an effective amount of a pharmaceutical combination of the application, or an effective amount of an allosteric EGFR inhibitor, as described herein, in combination with (e.g., in temporal proximity with) an effective amount of an ATP-competitive EGFR inhibitor, as described herein.

Another aspect of the present application relates to a method of treating or preventing cancer in a subject, wherein the subject is identified as being in need of EGFR inhibition for the treatment or prevention of cancer. The method comprises administering to the subject an effective amount of a pharmaceutical combination of the application, or an effective amount of an allosteric EGFR inhibitor, as described herein, in combination with (e.g., in temporal proximity with) an effective amount of an ATP-competitive EGFR inhibitor, as described herein.

Another aspect of the present application relates to a method of treating or preventing cancer, wherein the cell of the cancer comprises an activated ERBB2. The method comprises administering to a subject in need thereof an effective amount of a pharmaceutical combination of the application, or an effective amount of an allosteric EGFR inhibitor, as described herein, in combination with (e.g., in temporal proximity with) an effective amount of an ATP-competitive EGFR inhibitor, as described herein.

Another aspect of the present application relates to a method of treating or preventing cancer in a subject, wherein the subject is identified as being in need of ERBB2 inhibition for the treatment or prevention of cancer. The method comprises administering to the subject an effective amount of a pharmaceutical combination of the application, or an effective amount of an allosteric EGFR inhibitor, as described herein, in combination with (e.g., in temporal proximity with) an effective amount of an ATP-competitive EGFR inhibitor, as described herein.

Another aspect of the present application relates to an allosteric EGFR inhibitor, as described herein, for use in combination (e.g., in a combinational therapy) with an ATP-competitive EGFR inhibitor, as described herein, for

inhibiting a kinase (e.g., EGFR) in a subject in need thereof,

treating or preventing a disease (e.g., a disease in which EGFR plays a role) in a subject in need thereof,

treating or preventing a disease resistant to an EGFR targeted therapy, such as a therapy with gefitinib, erlotinib, afatinib, AZD9291, CO-1686, or WZ4002, in a subject in need thereof,

treating or preventing cancer in a subject in need thereof, wherein the cell of the cancer comprises an activated EGFR or an activated ERBB2, or

treating or preventing cancer in a subject, wherein the subject is identified as being in need of EGFR inhibition or ERBB2 inhibition for the treatment or prevention of cancer.

Another aspect of the present application relates to use of an allosteric EGFR inhibitor, as described herein, in combination (e.g., in a combinational therapy) with an ATP-competitive EGFR inhibitor, as described herein, for

inhibiting a kinase (e.g., EGFR) in a subject in need thereof,

treating or preventing a disease (e.g., a disease in which EGFR plays a role) in a subject in need thereof,

treating or preventing a disease resistant to an EGFR targeted therapy, such as a therapy with gefitinib, erlotinib, afatinib, AZD9291, CO-1686, or WZ4002, in a subject in need thereof,

treating or preventing cancer in a subject in need thereof, wherein the cell of the cancer comprises an activated EGFR or an activated ERBB2, or

treating or preventing cancer in a subject, wherein the subject is identified as being in need of EGFR inhibition or ERBB2 inhibition for the treatment or prevention of cancer.

Another aspect of the present application relates to a combination (e.g., a therapeutic combination) of an allosteric EGFR inhibitor, as described herein, and an ATP-competitive EGFR inhibitor, as described herein, for

inhibiting a kinase (e.g., EGFR) in a subject in need thereof,

treating or preventing a disease (e.g., a disease in which EGFR plays a role) in a subject in need thereof,

treating or preventing a disease resistant to an EGFR targeted therapy, such as a therapy with gefitinib, erlotinib, afatinib, AZD9291, CO-1686, or WZ4002, in a subject in need thereof,

treating or preventing cancer in a subject in need thereof, wherein the cell of the cancer comprises an activated EGFR or an activated ERBB2, or

treating or preventing cancer in a subject, wherein the subject is identified as being in need of EGFR inhibition or ERBB2 inhibition for the treatment or prevention of cancer.

Another aspect of the present application relates to use of a combination (e.g., a therapeutic combination) of an allosteric EGFR inhibitor, as described herein, and an ATP-competitive EGFR inhibitor, as described herein, in

inhibiting a kinase (e.g., EGFR) in a subject in need thereof,

treating or preventing a disease (e.g., a disease in which EGFR plays a role) in a subject in need thereof,

treating or preventing a disease resistant to an EGFR targeted therapy, such as a therapy with gefitinib, erlotinib, afatinib, AZD9291, CO-1686, or WZ4002, in a subject in need thereof,

treating or preventing cancer in a subject in need thereof, wherein the cell of the cancer comprises an activated EGFR or an activated ERB32, or

treating or preventing cancer in a subject, wherein the subject is identified as being in need of EGFR inhibition or ERBB2 inhibition for the treatment or prevention of cancer.

Another aspect of the present application relates to a combination (e.g., a therapeutic combination) of an allosteric EGFR inhibitor, as described herein, and an ATP-competitive EGFR inhibitor, as described herein, for use in the manufacture of a medicament for

inhibiting a kinase (e.g., EGFR) in a subject in need thereof,

treating or preventing a disease (e.g., a disease in which EGFR plays a role) in a subject in need thereof,

treating or preventing a disease resistant to an EGFR targeted therapy, such as a therapy with gefitinib, erlotinib, afatinib, AZD9291, CO-1686, or WZ4002, in a subject in need thereof,

treating or preventing cancer in a subject in need thereof, wherein the cell of the cancer comprises an activated EGFR or an activated ERB32, or

treating or preventing cancer in a subject, wherein the subject is identified as being in need of EGFR inhibition or ERBB2 inhibition for the treatment or prevention of cancer.

Another aspect of the present application relates to use of a combination (e.g., a therapeutic combination) of an allosteric EGFR inhibitor, as described herein, and an ATP-competitive EGFR inhibitor, as described herein, in the manufacture of a medicament for

inhibiting a kinase (e.g., EGFR) in a subject in need thereof,

treating or preventing a disease (e.g., a disease in which EGFR plays a role) in a subject in need thereof,

treating or preventing a disease resistant to an EGFR targeted therapy, such as a therapy with gefitinib, erlotinib, afatinib, AZD9291, CC-1686, or WZ4002, in a subject in need thereof,

treating or preventing cancer in a subject in need thereof, wherein the cell of the cancer comprises an activated EGFR or an activated ERBB2, or

treating or preventing cancer in a subject, wherein the subject is identified as being in need of EGFR inhibition or ERBB2 inhibition for the treatment or prevention of cancer.

Another aspect of the present application relates to a pharmaceutical combination of the application for

inhibiting a kinase (e.g., EGFR) in a subject in need thereof,

treating or preventing a disease (e.g., a disease in which EGFR plays a role) in a subject in need thereof,

treating or preventing a disease resistant to an EGFR targeted therapy, such as a therapy with gefitinib, erlotinib, afatinib, AZD9291, CO-1686, or WZ4002, in a subject in need thereof,

treating or preventing cancer in a subject in need thereof, wherein the cell of the cancer comprises an activated EGFR or an activated ERBB2, or

treating or preventing cancer in a subject, wherein the subject is identified as being in need of EGFR inhibition or ERBB2 inhibition for the treatment or prevention of cancer.

Another aspect of the present application relates to use of a pharmaceutical combination of the application for

inhibiting a kinase (e.g., EGFR) in a subject in need thereof,

treating or preventing a disease (e.g., a disease in which EGFR plays a role) in a subject in need thereof,

treating or preventing a disease resistant to an EGFR targeted therapy, such as a therapy with gefitinib, erlotinib, afatinib, AZD9291, CC-1686, or WZ4002, in a subject in need thereof,

treating or preventing cancer in a subject in need thereof, wherein the cell of the cancer comprises an activated EGFR or an activated ERBB2, or

treating or preventing cancer in a subject, wherein the subject is identified as being in need of EGFR inhibition or ERBB2 inhibition for the treatment or prevention of cancer.

Another aspect of the present application relates to a pharmaceutical combination of the application for use in the manufacture of a medicament for

inhibiting a kinase (e.g., EGFR) in a subject in need thereof,

treating or preventing a disease (e.g., a disease in which EGFR plays a role) in a subject in need thereof,

treating or preventing a disease resistant to an EGFR targeted therapy, such as a therapy with gefitinib, erlotinib, afatinib, AZD9291, CO-1686, or WZ4002, in a subject in need thereof,

treating or preventing cancer in a subject in need thereof, wherein the cell of the cancer comprises an activated EGFR or an activated ERBB2, or

treating or preventing cancer in a subject, wherein the subject is identified as being in need of EGFR inhibition or ERBB2 inhibition for the treatment or prevention of cancer.

Another aspect of the present application relates to use of a pharmaceutical combination of the application in the manufacture of a medicament for

inhibiting a kinase (e.g., EGFR) in a subject in need thereof,

treating or preventing a disease (e.g., a disease in which EGFR plays a role) in a subject in need thereof,

treating or preventing a disease resistant to an EGFR targeted therapy, such as a therapy with gefitinib, erlotinib, afatinib, AZD9291, CO-1686, or WZ4002, in a subject in need thereof,

treating or preventing cancer in a subject in need thereof, wherein the cell of the cancer comprises an activated EGFR or an activated ERBB2, or

treating or preventing cancer in a subject, wherein the subject is identified as being in need of EGFR inhibition or ERBB2 inhibition for the treatment or prevention of cancer.

The present application provides pharmaceutical combinations, kits, and methods to inhibit EGFR, such as EGFR containing one or more mutations, that are useful in the treatment or prevention of diseases such as cancer and metastasis. The present application further provides pharmaceutical combinations and kits with an improved efficacy and/or safety profile relative to known EGFR inhibitors.

The details of the application are set forth in the accompanying description below. Although methods and materials similar or equivalent to those described herein can be used in the practice or testing of the present application, illustrative methods and materials are now described. Other features, objects, and advantages of the application will be apparent from the description and from the claims. In the specification and the appended claims, the singular forms also include the plural unless the context clearly dictates otherwise. Unless defined otherwise, all technical and scientific terms used herein have the same meaning as commonly understood by one of ordinary skill in the art to which this application belongs. The contents of all references (including literature references, issued patents, published patent applications, and co-pending patent applications) cited throughout this application are hereby expressly incorporated herein in their entireties by reference.

### BRIEF DESCRIPTION OF THE DRAWINGS

FIG. 1A is a Western blot showing the levels of phosphorylated EGFR (pEGFR), EGFR, phosphorylated AKT (pAKT), AKT, phosphorylated ERK1/2 (pERK1/2), ERK1/2 and tubulin (as loading control) in H1975 cells harboring L85SR/T790M EGFR treated with DMSO, or with the indicated concentrations of Compound A or Compound O.

FIG. 1B is a plot showing the tumor growth from H1975 cells harboring L858R/T7901M EGFR implanted in animals treated with vehicle, Compound A, or Compound O.

FIG. 2A are Western blots showing the levels of EGFR, phosphorylated EGFR (pEGFR), and tubulin (as loading control). Ba/F3 cells harboring L858R/T790M EGFR were treated with DMSO, or with the indicated concentrations of WZ-4002 or Compound O. Lysates from the cells were either directly immunoblotted to detect EGFR, pEGFR, and tubulin, or first immunoprecipitated by biotinylated Compound A before immunoblotting for detection of EGFR, pEGFR, and tubulin. Unlike WZ-4002, binding of Compound O to EGFR does not interfere with binding of Compound A to EGFR.

FIG. 2B is a Western blot showing the levels of EGFR, phosphorylated EGFR (pEGFR), and tubulin (as loading control). H3255 GR cells harboring L858R/T790M EGFR were treated with DMSO, or with 1 μM Compound O. Lysates from the cells were either directly immunoblotted to detect EGFR, pEGFR, and tubulin, or first immunoprecipitated by biotinylated Compound A before immunoblotting for detection of EGFR, pEGFR, and tubulin.

FIG. 3A is a plot showing growth (% of DMSO treated control) of H3255 GR cells harboring L858R/T790M EGFR treated with the indicated concentrations of Compound A, Compound O (alone), or Compound O (in the presence of 10 μM Compound A).

FIG. 3B is a Western blot showing the levels of phosphorylated EGFR (pEGFR), EGFR, phosphorylated AKT (pAKT), AKT, phosphorylated ERK1/2 (pERK1/2), ERK1/2, and tubulin (as loading control) in H3255 GR cells harboring L858R/T790M EGFR treated with DMSO, or with the indicated concentrations of Compound O alone or Compound O in the presence of 1 μM or 10 μM Compound A, or with 1 μM or 10 μM Compound A.

FIG. 4A is a plot showing the level of apoptosis (measured by caspase activity) over time in H3255 GR cells harboring L8585R/T790M EGFR treated with DMSO, or with 10 μM Compound A, 0.1 μM Compound O, or a combination of 0.1 μM Compound O and 10 μM Compound A.

FIG. 4B is a plot showing confluency over time of cells treated with DMSO, or with 10 μM Compound A, 1 μM Compound O, or a combination of 0.1 μM Compound and 10 μM Compound A.

FIG. 5 is quantitative analyses of resistant colonies that emerged after continuous treatment with 1 μM of Compound O alone, 1 μM of gefitinib alone, 10 μM of Compound A alone, or Compound A in combination with either Compound O or gefitinib for two weeks in ENU-treated L858R and L858R/T790M Ba/F3 cells. Data is shown as the percentage of resistant colonies relative to the total number of colonies (300) treated over time.

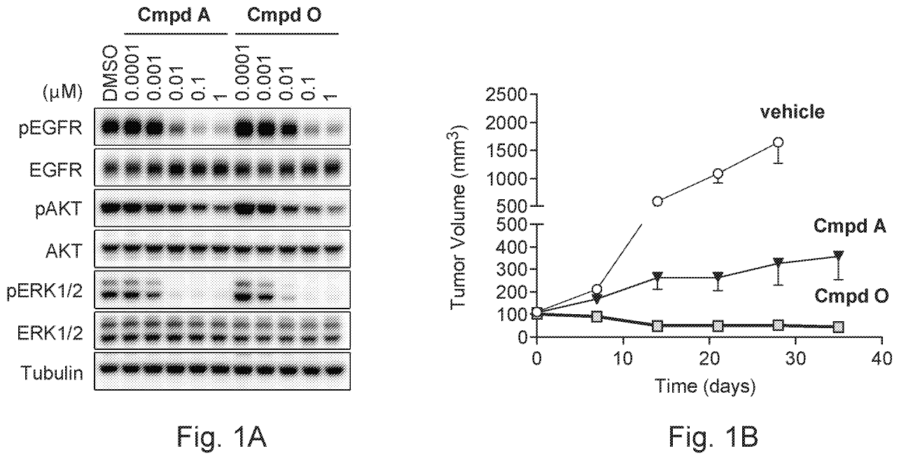

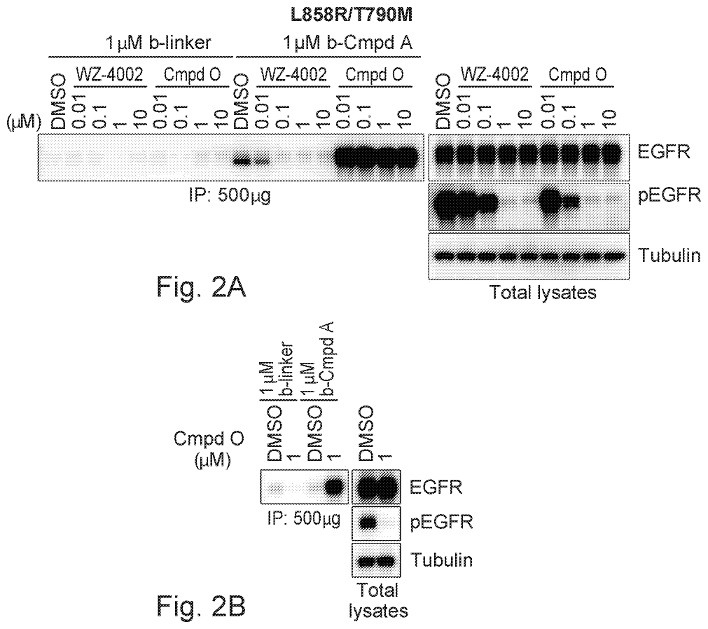

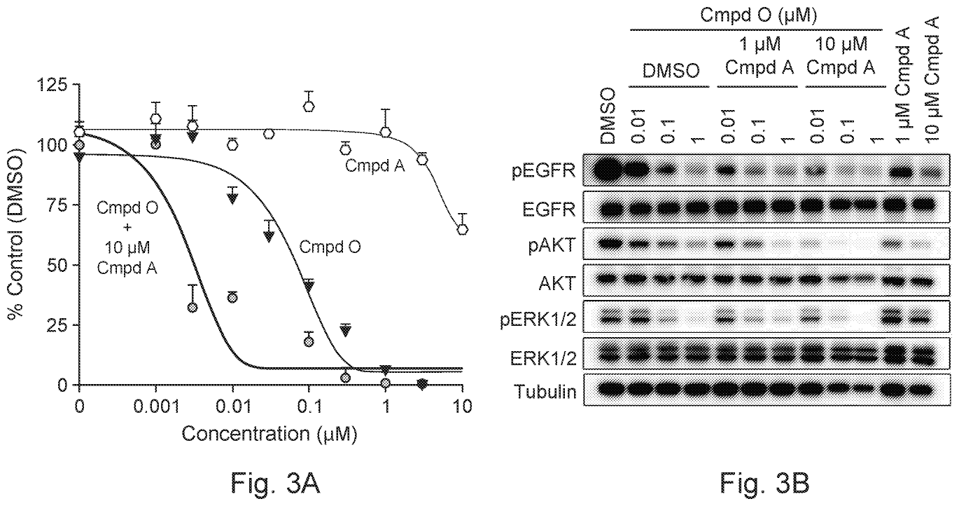

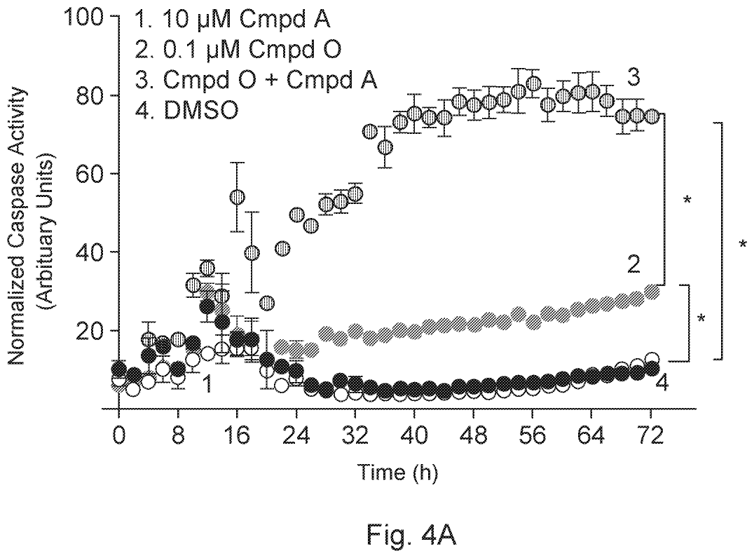

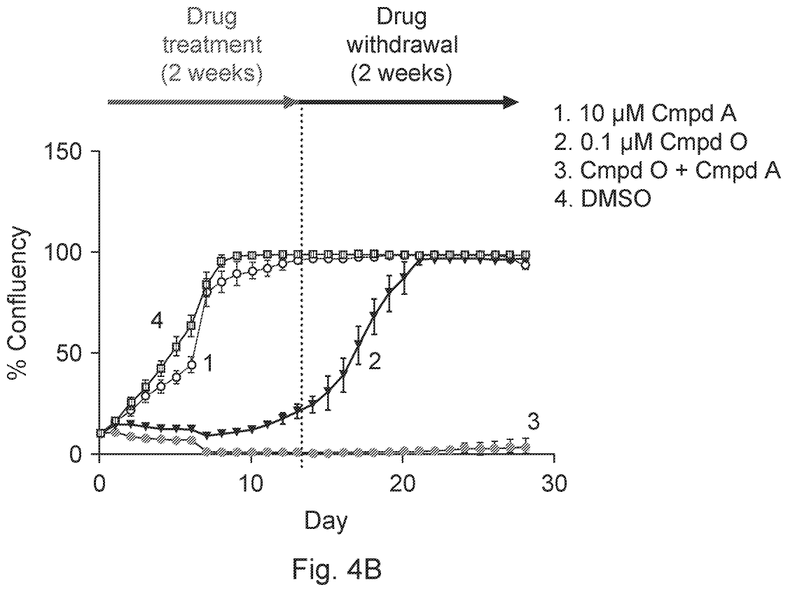

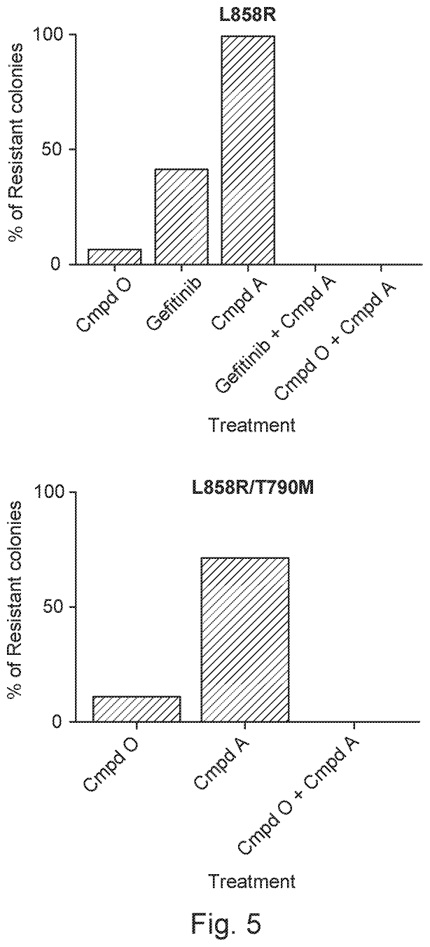

### DETAILED DESCRIPTION

### Pharmaceutical Combinations of the Application

The present application relates to a pharmaceutical combination comprising an allosteric EGFR inhibitor and an ATP-competitive EGFR inhibitor.

In one embodiment, the allosteric EGFR inhibitor is a compound of Formula I:

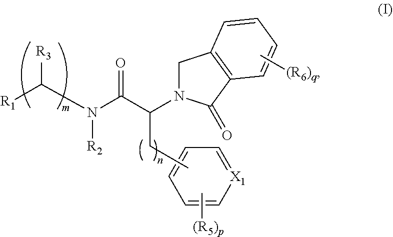

or a pharmaceutically acceptable salt, hydrate, or solvate thereof, wherein:

R₁ is C₆-C₁₀ aryl, or heteroaryl comprising one or two 5- to 7-membered rings and 1-4 heteroatoms selected from N, O, and S, wherein the aryl or heteroaryl is optionally substituted with one or more R₁₁;
each R₁ is independently C₁-C₄ alkyl, C₁-C₄ haloalkyl, C₁-C₄ alkoxy, C₁-C₄ haloalkoxy, halogen, NO₂, OH, CN, C(O)R₁₃, C(O)OR₁₃, C(O)NR₁₃R₁₄, NR₁₃R₁₄, C₃-C₇ cycloalkyl, heterocyclyl comprising one 5- to 7-membered ring and 1-3 heteroatoms selected from N, O, and S, C₆-C₁₀ aryl, or heteroaryl comprising one or two 5- to 7-membered rings and 1-4 heteroatoms selected from N, O, and S, wherein the alkyl, cycloalkyl, heterocyclyl, aryl, or heteroaryl is optionally substituted with one or more R₁₂;
each R₁₂ is independently C₁-C₄ alkyl, C₁-C₄ haloalkyl, C₁-C₄ alkoxy, C₁-C₄ haloalkoxy, halogen, NO₂, OH, CN, C₃-C₇ cycloalkyl, heterocyclyl comprising one 5- to 7-membered ring and 1-3 heteroatoms selected from N, O, and S, C₆-C₁₀ aryl, or heteroaryl comprising one or two 5- to 7-membered rings and 1-4 heteroatoms selected from N, O, and S, wherein the aryl or heteroaryl is optionally substituted with one or more substituents independently selected from C₁-C₄ alkyl, C₁-C₄ alkoxy, C₁-C₄ haloalkyl, C₁-C₄ haloalkoxy, halogen, NH₂, NH(C₁-C₄) alkyl, N((C₁-C₄) alkyl)₂, C₃-C₇ cycloalkyl, and heterocyclyl comprising one 5- to 7-membered ring and 1-3 heteroatoms selected from N, O, and S;
each R₁₃ is independently H, C₁-C₄ alkyl, C₃-C₇ cycloalkyl, or heterocyclyl comprising one 5- to 7-membered ring and 1-3 heteroatoms selected from N, O, and S, wherein the alkyl, cycloalkyl, or heterocyclyl is optionally substituted with one or more substituents independently selected from C₁-C₄ alkyl, halogen, OH, NH₂, NH(C₁-C₄) alkyl, N((C₁-C₄) alkyl)₂, and heterocyclyl comprising one 5- to 7-membered ring and 1-3 heteroatoms selected from N, O, and S;

each R₁₄ is independently H or C₁-C₃ alkyl;

R₂ is H or C₁-C₃ alkyl;

R₃ is H or C₁-C₃ alkyl;

X₁ is N or CR₄;

R₄ is H, C₁-C₄ alkyl, C₁-C₄ haloalkyl, C₁-C₄ alkoxy, C₁-C₄ haloalkoxy, halogen, NO₂, NH₂, OH, or CN;

each R₅ is independently C₁-C₄ alkyl, C₁-C₄ haloalkyl, C₁-C₄ alkoxy, C₁-C₄ haloalkoxy, halogen, NO₂, NH₂, OH, or CN;

each R₆ is independently halogen, C₃-C₇ cycloalkyl, C₄-C₇ cycloalkenyl, C₆-C₁₀ aryl, NH(C₆-C₁₀) aryl, or heteroaryl comprising one or two 5- to 7-membered rings and 1-4 heteroatoms selected from N, O, and S, wherein the aryl or heteroaryl is optionally substituted with one or more R₇;

each R₇ is independently C₁-C₄ alkyl, C₁-C₄ alkoxy, C₁-C₄ haloalkyl, C₁-C₄ haloalkoxy, halogen, C(O)OH, C(O)O(C₁-C₄) alkyl, C(O)NR₈R₉, NH₂, OH, CN, O(CH₂)₀₋₃—(C₆-C₁₀) aryl, or (CH₂)₀₋₃-heterocyclyl which comprises one 5- to 7-membered ring and 1-3 heteroatoms selected from N, O, and S, wherein the heterocyclyl is optionally substituted with one or more substituents independently selected from C₁-C₄ alkyl, C₁-C₄ alkoxy, C₁-C₄ haloalkyl, C₁-C₄ haloalkoxy, halogen, and C(O)O((C₁-C₄) alkyl);

R₈ is H or C₁-C₃ alkyl;

R₉ is H or C₁-C₄ alkyl optionally substituted with one or more substituents independently selected from NH₂, NH(C₁-C₄) alkyl, N((C₁-C₄) alkyl)₂, and heterocyclyl comprising one 5- to 7-membered ring and 1-3 heteroatoms selected from N, O, and S; or

R₈ and R₉ together with the nitrogen atom to which they are attached form a 5- or 6-membered heterocyclyl optionally containing 1-2 additional heteroatoms selected from N, O, and S;

m and n are each independently 0 or 1;

q is 0, 1, or 2; and

p is 0, 1, 2, 3 or 4,

provided that when m is 0, n is 0, p is 0, q is 0, and X₁ is CH, then R₁ is not

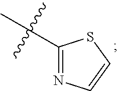 [SMILES: *c1nccs1]

and

that when p is 2, X₁ is CH, and one R₅ is 4-fluoro, then the other R₅ is not 2-hydroxy.

In one embodiment, a compound of Formula I is of Formula II or III:

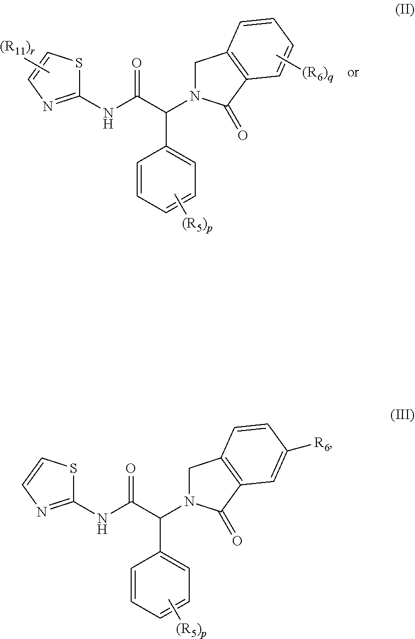

or a pharmaceutically acceptable salt, hydrate, or solvate thereof, wherein:

R₅, R₆, R₇, R₈, R₉, R₁₁, R₁₂, R₁₃, R₁₄, p, and q are each as defined in Formula I;

r is 0, 1, or 2,

provided that p, q, and r are not all 0.

For a compound of Formula I, II, or III, where applicable:

(I1) In one embodiment, R₂ is H.

(I2) In one embodiment, R₂ is C₁-C₃ alkyl (e.g., methyl, ethyl propyl, or i-propyl). In one embodiment, R₂ is methyl. In one embodiment, R₂ is ethyl.

(II1) In one embodiment, R₃ is H.

(II2) In one embodiment, R₃ is C₁-C₃ alkyl (e.g., methyl, ethyl, propyl, or i-propyl). In one embodiment, R₃ is methyl. In one embodiment, R₃ is ethyl.

(III1) In one embodiment, X₁ is N.

(III2) In one embodiment, X₁ is CR₄.

(IV1) In one embodiment, R₄ is H.

(IV2) In one embodiment, R₄ is C₁-C₄ alkyl (e.g., methyl, ethyl, propyl, i-propyl, n-butyl, i-butyl, s-butyl, or t-butyl), or C₁-C₄ haloalkyl (e.g., methyl, ethyl, propyl, i-propyl, n-butyl, i-butyl, s-butyl, or t-butyl, each of which is substituted with one or more halogen (e.g., F, Cl, Br, or I)).

(IV3) In one embodiment, R₄ is C₁-C₄ alkoxy (e.g., methoxy, ethoxy, n-propoxy, i-propoxy, n-butoxy, i-butoxy, s-butoxy, or t-butoxy), or C₁-C₄ haloalkoxy (e.g., methoxy, ethoxy, n-propoxy, i-propoxy, n-butoxy, i-butoxy, s-butoxy, or t-butoxy, each of which is substituted with one or more halogen (e.g., F, Cl, Br, or I)).

(IV4) In one embodiment, R₄ is halogen (e.g., F, Cl, Br, or I). In one embodiment, R₄ is F or Cl. In a further embodiment, R₄ is F.

(IV5) In one embodiment, R₄ is NO₂, NH₂, OH, or CN. In one embodiment, R₄ is NO₂ or NH₂.

(V1) In one embodiment, at least one R₅ is C₁-C₄ alkyl (e.g., methyl, ethyl, propyl, i-propyl, n-butyl, i-butyl, s-butyl, or t-butyl), or C₁-C₄ haloalkyl (e.g., methyl, ethyl, propyl, i-propyl, n-butyl, i-butyl, s-butyl, or t-butyl, each of which is substituted with one or more halogen (e.g., F, Cl, Br, or I)).

(V2) In one embodiment, at least one R₅ is C₁-C₄ alkoxy (e.g., methoxy, ethoxy, n-propoxy, i-propoxy, n-butoxy, i-butoxy, s-butoxy, or t-butoxy), or C₁-C₄ haloalkoxy (e.g., methoxy, ethoxy, n-propoxy, i-propoxy, n-butoxy, i-butoxy, s-butoxy, or t-butoxy, each of which is substituted with one or more halogen (e.g., F, Cl, Br, or I)).

(V3) In one embodiment, at least one R₅ is halogen (e.g., F, Cl, Br, or I), NO₂, NH₂, OH, or CN. In one embodiment, at least one R₅ is F or Cl. In one embodiment, at least one R₅ is F.

(V4) In one embodiment, at least one R₅ is NO₂, NH), OH, or CN. In one embodiment, at least one R₅ is NO₂ or NH₂.

(V5) In one embodiment, at least one R is halogen (e.g., F, Cl, Br, or I) and at least one R₅ is OH.

(V6) In one embodiment, one R₅ is halogen (e.g., F, Cl, Br, or I) and one R₅ is OH.

(VI1) In one embodiment, at least one R₆ is halogen (e.g., F, Cl, Br, or I). In one embodiment, at least one R₆ is F, Cl, or Br. In one embodiment, at least one R₆ is F. In one embodiment, at least one R₆ is Cl. In one embodiment, at least one R₆ is Br.

(V12) In one embodiment, at least one R₆ is C₃-C₇ cycloalkyl (e.g., cyclopropyl, cyclobutyl, cyclopentyl, cyclohexyl, or cycloheptyl).

(VI3) In one embodiment, at least one R₆ is C₄-C₇ cycloalkenyl (e.g., cyclobutenyl, cyclopentenyl, cyclohexenyl, or cycloheptenyl).

(VI4) In one embodiment, at least one R₆ is C₆-C₁₀ aryl optionally substituted with one or more R₇. In one embodiment, at least one R₆ is phenyl optionally substituted with one or more R₇. In one embodiment, at least one R₆ is phenyl optionally substituted with one to three R₇. In one embodiment, at least one R₆ is phenyl.

(VI5) In one embodiment, at least one R₆ is NH—(C₆-C₁₀) aryl optionally substituted with one or more R₇. In one embodiment, at least one R₆ is NH-phenyl optionally substituted with one or more R₇. In one embodiment, at least one R₆ is NH-phenyl optionally substituted with one to three R₇. In one embodiment, at least one R₆ is NH-phenyl.

(V16) In one embodiment, at least one R₆ is heteroaryl comprising one or two 5- to 7-membered rings and 1-4 heteroatoms selected from N, O, and S (e.g., pyrrolyl, pyrazolyl, imidazolyl, triazolyl, oxazolyl, isoxazolyl, oxadiazolyl, dioxazolyl, thiazolyl, isothiazolyl, thiadiazolyl, dithiazolyl, thiophenyl, pyridinyl, pyridazinyl, pyrimnidinyl, triazinyl, indolyl, quinolinyl, isoquinolinyl, benzothiazolyl, benzoimidazolyl, benzooxazolyl, thiazolopyridinyl, pyrazolopyrimidinyl, etc.) optionally substituted with one or more R₇. In one embodiment, at least one R₆ is pyrazolyl, thiophenyl, pyridinyl, pyrimidinyl, indolyl, or quinolinyl, each optionally substituted with one or more R₇.

(VII1) In one embodiment, at least one R₇ is C₁-C₄ alkyl (e.g., methyl, ethyl, propyl, i-propyl, n-butyl, i-butyl, s-butyl, or t-butyl), or C₁-C₄ haloalkyl (e.g., methyl, ethyl, propyl, i-propyl, n-butyl, i-butyl, s-butyl, or t-butyl, each of which is substituted with one or more halogen (e.g., F, Cl, Br, or I)). In a further embodiment, at least one R₇ is methyl or ethyl. In a further embodiment, at least one R₇ is CF₃.

(VII2) In one embodiment, at least one R₇ is C₁-C₄ alkoxy (e.g., methoxy, ethoxy, n-propoxy, i-propoxy, n-butoxy, i-butoxy, s-butoxy, or t-butoxy), or C₁-C₄ haloalkoxy (e.g., methoxy, ethoxy, n-propoxy, i-propoxy, n-butoxy, i-butoxy, s-butoxy, or t-butoxy, each of which is substituted with one or more halogen (e.g., F, Cl, Br, or I)). In a further embodiment, at least one R₇ is methoxy. In a further embodiment, at least one R₇ is OCF₃.

(VII3) In one embodiment, at least one R₇ is halogen (e.g., F, Cl, Br, or 1). In a further embodiment, at least one R₇ is F.

(VII4) In one embodiment, at least one R₇ is C(O)OH or C(O)O(C₁-C₄) alkyl (e.g., methyl, ethyl, propyl, i-propyl, n-butyl, i-butyl, s-butyl, or t-butyl). In a further embodiment, at least one R₇ is C(O)OH or C(O)OCH₃.

(VII5) In one embodiment, at least one R₇ is NH₂, OH, or CN.

(VII6) In one embodiment, at least one R₇ is C(O)NR₈R₉.

(VII7) In one embodiment, at least one R₇ is O(CH₂)₀₋₃—(C₆-C₁₀) aryl. In a further embodiment, at least one R₇ is OCH₂-phenyl.

(VII8) In one embodiment, at least one R₇ is (CH₂)₀₋₃-heterocyclyl which comprises one 5- to 7-membered ring and 1-3 heteroatoms selected from N, O, and S, wherein the heterocyclyl is selected from pyrrolidinyl, pyrazolidinyl, imidazolidinyl, triazolidinyl, oxazolidinyl, isoxazolidinyl, oxadiazolidinyl, dioxazolidinyl, thiazolidinyl, isothiazolidinyl, thiadiazolidinyl, dithiazolidinyl, piperidinyl, piperazinyl, hexahydropyridazinyl, hexahydropyrimidinyl, morpholinyl, dioxanyl, azepinyl, diazepinyl, etc., and is optionally substituted with one or more substituents independently selected from C₁-C₄ alkyl (e.g., methyl, ethyl, propyl, i-propyl, n-butyl, i-butyl, s-butyl, ort-butyl), C₁-C₄ alkoxy (e.g., methoxy, ethoxy, n-propoxy, i-propoxy, n-butoxy, i-butoxy, s-butoxy, or t-butoxy), C₁-C₄ haloalkyl (e.g., methyl, ethyl, propyl, i-propyl, n-butyl, i-butyl, s-butyl, or t-butyl, each of which is substituted with one or more halogen (e.g., F, Cl, Br, or I), such as CH₂F, Cf₂, or CF), C₁-C₄ haloalkoxy (e.g., methoxy, ethoxy, n-propoxy, i-propoxy, n-butoxy, i-butoxy, s-butoxy, or t-butoxy, each of which is substituted with one or more halogen (e.g., F, Cl, Br, or I), such as OCH₂F, OCHF₂, or OCF₃), halogen (e.g., F, Cl, Br, or I), and C(O)O(C₁-C₄) alkyl (e.g., methyl, ethyl, propyl, i-propyl, n-butyl, i-butyl, s-butyl, or t-butyl). In one embodiment, at least one R₇ is (C₂)₀₋₁-heterocycle optionally substituted as described herein. In one embodiment, at least one R₇ is CH₂-pyrrolidinyl, CH₂-piperazinyl, pyrrolidinyl, morpholinyl, or piperazinyl, each optionally substituted as described herein. In one embodiment, at least one R₇ is C₂-pyrrolidinyl, CH-piperazinyl, pyrrolidinyl, morpholinyl, or piperazinyl, each optionally substituted with one or more C₁-C₄ alkyl (e.g., methyl, ethyl, propyl, i-propyl, n-butyl, i-butyl, s-butyl, or t-butyl), or C(O)O(C₁-C₄) alkyl (e.g., methyl, ethyl, propyl, i-propyl, n-butyl, i-butyl, s-butyl, or t-butyl).

(VIII1) In one embodiment, R₈ is H.

(VIII2) In one embodiment, R₈ is C₁-C₃ alkyl (e.g., methyl, ethyl, propyl, or i-propyl). In one embodiment, R₈ is methyl. In one embodiment, R₈ is ethyl.

(IX1) In one embodiment, R₉ is H.

(IX2) In one embodiment, R₉ is C₁-C₄ alkyl (e.g., methyl, ethyl, propyl, i-propyl, n-butyl, i-butyl, s-butyl, or t-butyl) optionally substituted with one or more substituents independently selected from NH₂, NH(C₁-C₄) alkyl (e.g., methylamino, ethylamino, propylamino, or butylamino), N((C₁-C₄) alkyl)₂ (e.g., dimethylamino, diethylamino, dipropylamino, or dibutylamino), and heterocyclyl comprising one 5- to 7-membered ring and 1-3 heteroatoms selected from N, O, and S (e.g. pyrrolidinyl pyrazolidinyl, imidazolidinyl, triazolidinyl, oxazolidinyl, isoxazolidinyl, oxadiazolidinyl, dioxazolidinyl, thiazolidinyl, isothiazolidinyl, thiadiazolidinyl, dithiazolidinyl, piperidinyl, hexahydropyridazinyl, hexahydropyrimidinyl, morpholinyl, dioxanyl, etc.). In one embodiment, R₉ is C₁-C₄ alkyl (e.g., methyl, ethyl, propyl, i-propyl, n-butyl, i-butyl, s-butyl, or t-butyl) optionally substituted with one to two substituents independently selected from NH₂, NH(C₁-C₄) alkyl, N((C₁-C₄) alkyl)₂, and 6-membered heterocycle comprising 1-3 heteroatoms selected from N, O, and S.

(IX3) In one embodiment, R₈ and R₉ together with the nitrogen atom to which they are attached form a 5-membered heterocyclyl optionally containing 1-2 additional heteroatoms selected from N, O, and S. In one embodiment, R₈ and R₉ together with the nitrogen atom to which they are attached form a 6-membered heterocycle optionally containing 1-2 additional heteroatoms selected from N, O, and S.

(X1) In one embodiment, R₁ is C₆-C₁₀ aryl optionally substituted with one or more R₁₁. In one embodiment, R₁ is phenyl optionally substituted with one or more R₁₁.

(X2) In one embodiment, R₁ is heteroaryl comprising one or two 5- to 7-membered rings and 1-4 heteroatoms selected from N, O, and S (e.g., pyrrolyl, pyrazolyl, imidazolyl, triazolyl, oxazolyl, isoxazolyl, oxadiazolyl, dioxazolyl, thiazolyl, isothiazolyl, thiadiazolyl, dithiazolyl, thiophenyl, pyridinyl, pyridazinyl, pyrimidinyl, triazinyl, benzothiazolyl, benzoimidazolyl, benzooxazolyl, quinolinyl, thiazolopyridinyl, pyrazolopyrimidinyl, etc.) optionally substituted with one or more R₁₁. In one embodiment, R₁ is heteroaryl comprising one 5-membered ring and 1-3 heteroatoms selected from N, O, and S, optionally substituted with one or more R₁₁. In one embodiment, R₁ is heteroaryl comprising one 6-membered ring and 1-3 heteroatoms selected from N, O, and S, optionally substituted with one or more R₁₁. In one embodiment, R₁ is heteroaryl comprising a 5-membered ring fused with a 6-membered ring and 1-4 heteroatoms selected from N, O, and S, optionally substituted with one or more R₁₁. In one embodiment, R₁ is selected from:

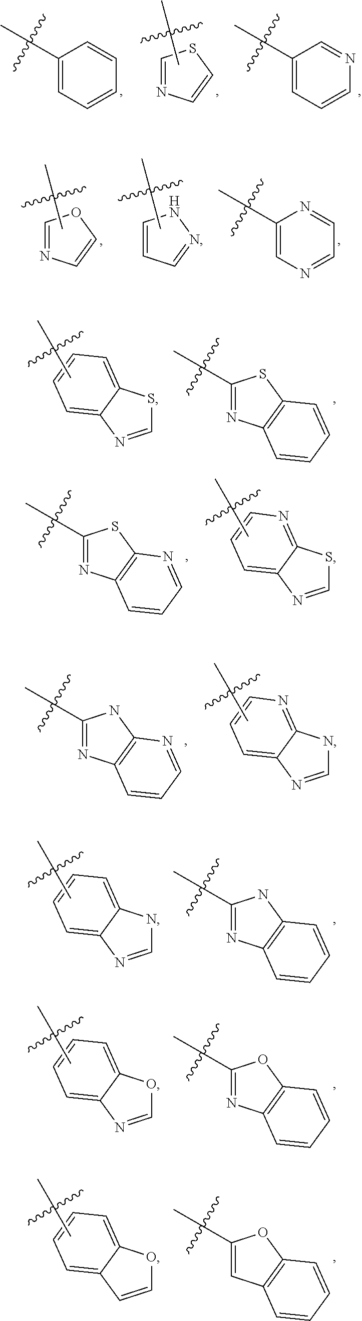

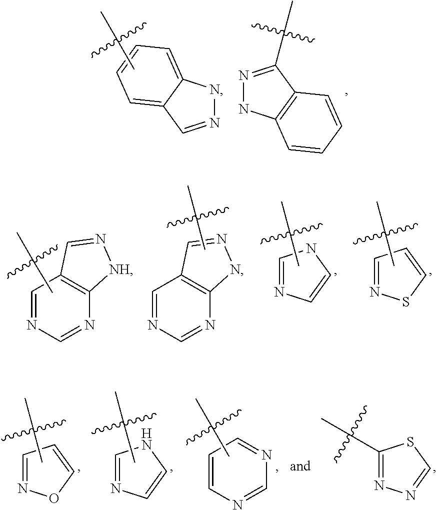

wherein each moiety is optionally substituted with one or more R₁₁. In one embodiment, R₁ is selected from:

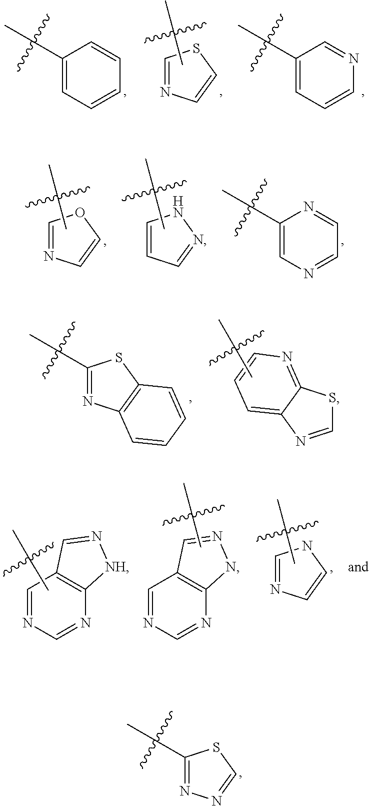

wherein each moiety is optionally substituted with one or more R₁₁. In one embodiment, R₁ is

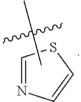

(XI1) In one embodiment, at least one R₁₁ is C₁-C₄ alkyl (e.g., methyl, ethyl, propyl, i-propyl, n-butyl, i-butyl, s-butyl, or t-butyl) optionally substituted with one or more R₁₂. In one embodiment, at least one R₁₁ is methyl.

(XI2) In one embodiment, at least one R₁₁ is C₁-C₄ haloalkyl (e.g., methyl, ethyl, propyl, i-propyl, n-butyl, i-butyl, s-butyl, or t-butyl, each of which is substituted with one or more halogen (e.g., F, Cl, Br, or I), such as CH₂F, CHF₂, or CF₃). In a further embodiment, at least one R₁ is CF₃.

(XI3) In one embodiment, at least one R₁₁ is C₁-C₄ alkoxy(e.g., methoxy, ethoxy, n-propoxy, i-propoxy, n-butoxy, i-butoxy, s-butoxy, or t-butoxy), or C₁-C₄ haloalkoxy (e.g., methoxy, ethoxy, n-propoxy, i-propoxy, n-butoxy, i-butoxy, s-butoxy, or t-butoxy, each of which is substituted with one or more halogen (e.g., F, Cl, Br, or I), such as OCH₂F, OCF₂, or OCF₃).

(XI4) In one embodiment, at least one R₁₁ is halogen (e.g., F, Cl, Br, or I). In one embodiment, at least one R₁₁ is F. In one embodiment, at least one R₁₁ is Cl. In one embodiment, at least one R₁₁ is Br.

(XI5) In one embodiment, at least one R₁₁ is NO₂, OH, or CN.

(X16) In one embodiment, at least one R₁₁ is C(O)R₁₃ or C(O)OR₁₃. In one embodiment, at least one R₁₁ is C(O)OCH₂CH₃.

(X17) In one embodiment, at least one R₁₁ is C(O)NR₁₃R₁₄ or NR₁₃R₁₄. In one embodiment, at least one R₁₁ is C(O)NR₁₃R₁₄ or NH₂.

(XI8) In one embodiment, at least one R₁₁ is C₃-C₇ cycloalkyl (e.g., cyclopropyl, cyclobutyl, cyclopentyl, cyclohexyl, or cycloheptyl) optionally substituted with one or more R₁₂.

(XI9) In one embodiment, at least one RH is heterocyclyl comprising one 5- to 7-membered ring and 1-3 heteroatoms selected from N, O, and S (e.g., pyrrolidinyl, pyrazolidinyl, imidazolidinyl, triazolidinyl, oxazolidinyl, isoxazolidinyl, oxadiazolidinyl, dioxazolidinyl, thiazolidinyl, isothiazolidinyl, thiadiazolidinyl, dithiazolidinyl, piperidinyl, hexahydropyridazinyl, hexahydropyrimidinyl, morpholinyl, dioxanyl, azepinyl, diazepinyl, etc.) optionally substituted with one or more R₁₂.

(XI10) In one embodiment, at least one R₁₁ is C₆-C₁₀ aryl optionally substituted with one or more R₁₂. In a further embodiment, at least one R₁₁ is phenyl optionally substituted with one or more R₁₂.

(XI11) In one embodiment, at least one R₁₁ is heteroaryl comprising one or two 5- to 7-membered rings and 1-4 heteroatoms selected from N, O, and S (e.g., pyrrolyl, pyrazolyl, imidazolyl, triazolyl, oxazolyl, isoxazolyl, oxadiazolyl, dioxazolyl, thiazolyl, isothiazolyl, thiadiazolyl, dithiazolyl, thiophenyl, pyridinyl, pyridazinyl, pyrimidinyl, triazinyl, benzothiazolyl, benzoimidazolyl, benzooxazolyl, quinolinyl, etc.) comprising 1-3 heteroatoms selected from N, O, and S, optionally substituted with one or more R₁₂. In one embodiment, at least one R₁₁ is heteroaryl comprising one 6-membered ring (e.g., pyridinyl, pyridazinyl, pyrimidinyl, triazinyl, etc.) optionally substituted with one or more R₁₂. In one embodiment, at least one R₁₁ is pyridinyl optionally substituted with one or more R₁₂. In a further embodiment, at least one R₁₁ is pyridinyl.

(XII1) In one embodiment, at least one R₁₂ is C₁-C₄ alkyl (e.g., methyl, ethyl, propyl, i-propyl, n-butyl, i-butyl, s-butyl, or t-butyl).

(XII2) In one embodiment, at least one R₁₂ is C₁-C₄ haloalkyl (e.g., methyl, ethyl, propyl, i-propyl, n-but, i-butyl, s-butyl, or t-butyl, each of which is substituted with one or more halogen (e.g., F, Cl, Br, or I), such as CH₂F, CHF₂, or CF₃).

(XII3) In one embodiment, at least one R₁₂ is C₁-C₄ alkoxy (e.g., methoxy, ethoxy, n-propoxy, i-propoxy, n-butoxy, i-butoxy, s-butoxy, or t-butoxy), or C₁-C₄haloalkoxy (e.g., methoxy, ethoxy, n-propoxy, i-propoxy, n-butoxy, i-butoxy, s-butoxy, or t-butoxy, each of which is substituted with one or more halogen (e.g., F, Cl, Br, or I), such as OCH₂F, OCHF₂, Or OCF₃).

(XII4) In one embodiment, at least one R₁₂ is halogen (e.g., F, Cl, Br, or I).

(XII5) In one embodiment, at least one R₁₂ is NO₂, OH, or CN.

(XII6) In one embodiment, at least one R₁₂ is C₃-C₇ cycloalkyl (e.g., cyclopropyl, cyclobutyl, cyclopentyl, cyclohexyl, or cycloheptyl).

(XII7) In one embodiment, at least one R₁₂ is heterocyclyl comprising one 5- to 7-membered ring and 1-3 heteroatoms selected from N, O, and S (e.g., pyrrolidinyl, pyrazolidinyl, imidazolidinyl, triazolidinyl, oxazolidinyl, isoxazolidinyl, oxadiazolidinyl, dioxazolidinyl, thiazolidinyl, isothiazolidinyl, thiadiazolidinyl, dithiazolidinyl, piperidinyl, hexahydropyridazinyl, hexahydropyrimidinyl, morpholinyl, dioxanyl, azepinyl, diazepinyl, etc.).

(XII8) In one embodiment, at least one R₁₂ is C₆-C₁₀ aryl optionally substituted with one or more substituents independently selected from C₁-C₄ alkyl (e.g., methyl, ethyl, propyl, i-propyl, n-butyl, i-butyl, s-butyl, or t-butyl), C₁-C₄ alkoxy (e.g., methoxy, ethoxy, n-propoxy, i-propoxy, n-butoxy, i-butoxy, s-butoxy, or t-butoxy), C₁-C₄ haloalkyl (e.g., methyl, ethyl, propyl, i-propyl, n-butyl, i-butyl, s-butyl, or t-butyl, each of which is substituted with one or more halogen (e.g., F, Cl, Br, or I), such as CH₂F, CHF₂, or CF₃), C₁-C₄ haloalkoxy (e.g., methoxy, ethoxy, n-propoxy, i-propoxy, n-butoxy, i-butoxy, s-butoxy, or t-butoxy, each of which is substituted with one or more halogen (e.g., F, Cl, Br, or I), such as OCH₂F, OCHF₂, or OCF₃), halogen (e.g., F, Cl, Br, or I), NH₂, NH(C₁-C₄) alkyl (e.g., methylamino, ethylamino, propylamino, or butylamino), N((C₁-C₄) alkyl)₂ (e.g., dimethylamino, diethylamino, dipropylamino, or dibutylamino), C₃-C₇, cycloalkyl (e.g., cyclopropyl, cyclobutyl, cyclopentyl, cyclohexyl, or cycloheptyl), and heterocyclyl comprising one 5- to 7-membered ring and 1-3 heteroatoms selected from N, O, and S (e.g., pyrrolidinyl, pyrazolidinyl, imidazolidinyl, triazolidinyl, oxazolidinyl, isoxazolidinyl, oxadiazolidinyl, dioxazolidinyl, thiazolidinyl, isothiazolidinyl, thiadiazolidinyl, dithiazolidinyl, piperidinyl, hexahydropyridazinyl, hexahydropyrimidinyl, morpholinyl, dioxanyl, azepinyl, diazepinyl, etc.).

(XII9) In one embodiment, R₁₂ is heteroaryl comprising one or two 5- to 7-membered rings and 1-4 heteroatoms selected from N, O, and S (e.g., pyrrolyl, pyrazolyl, imidazolyl, triazolyl, oxazolyl, isoxazolyl, oxadiazolyl, dioxazolyl, thiazolyl, isothiazolyl, thiadiazolyl, dithiazolyl, thiophenyl, pyridinyl, pyridazinyl, pyrimidinyl, triazinyl, benzothiazolyl, benzoimidazolyl, benzooxazolyl, quinolinyl, etc.) optionally substituted with one or more substituents independently selected from C₁-C₄ alkyl (e.g., methyl, ethyl, propyl, i-propyl, n-butyl, i-butyl, s-butyl, or t-butyl), C₁-C₄ alkoxy (e.g., methoxy, ethoxy, n-propoxy, i-propoxy, n-butoxy, i-butoxy, s-butoxy, or t-butoxy), C₁-C₄ haloalkyl (e.g., methyl, ethyl, propyl, i-propyl, n-butyl, i-butyl, s-butyl, or t-butyl, each of which is substituted with one or more halogen (e.g., F, Cl, Br, or I), such as CH₂F, CF₂, Or CF₃), C₁-C₄ haloalkoxy (e.g., methoxy, ethoxy, n-propoxy, i-propoxy, n-butoxy, i-butoxy, s-butoxy, or t-butoxy, each of which is substituted with one or more halogen (e.g., F, Cl, Br, or I), such as OCH₂F, OCHF₂, or OCF₃), halogen (e.g., F, Cl, Br, or I), NH₂, NH(C₁-C₄) alkyl (e.g., methylamino, ethylamino, propylamino, or butylamino), N((C₁-C₄) alkyl)₂ (e.g., dimethylamino, diethylamino, dipropylamino, or dibutylamino), C₃-C₂ cycloalkyl (e.g., cyclopropyl, cyclobutyl, cyclopentyl, cyclohexyl, or cycloheptyl), and heterocyclyl comprising a 5- to 7-membered ring and 1-3 heteroatoms selected from N, O, and S (e.g., pyrrolidinyl, pyrazolidinyl, imidazolidinyl, triazolidinyl, oxazolidinyl, isoxazolidinyl, oxadiazolidinyl, dioxazolidinyl, thiazolidinyl, isothiazolidinyl, thiadiazolidinyl, dithiazolidinyl, piperidinyl, hexahydropyridazinyl, hexahydropyrimidinyl, morpholinyl, dioxanyl, azepinyl, diazepinyl, etc.). In one embodiment, at least one R₁₂ is heteroaryl comprising one 5-membered ring fused with a 6-membered ring and 1-4 heteroatoms selected from N, O, and S, optionally substituted with one or more substituents independently selected from NH₂, NH(C₁-C₄) alkyl (e.g., methylamino, ethylamino, propylamino, or butylamino), N((C₁-C₄) alkyl)₂ (e.g., dimethylamino, diethylamino, dipropylamino, or dibutylamino), and C₃-C₇ cycloalkyl (e.g., cyclopropyl, cyclobutyl, cyclopentyl, cyclohexyl, or cycloheptyl).

(XIII1) In one embodiment, at least one R₁₃ is II.

(XIII2) In one embodiment, at least one R₁₃ is C₁-C₄ alkyl (e.g., methyl, ethyl, propyl, i-propyl, n-butyl, i-butyl, s-butyl, or t-butyl) optionally substituted with one or more substituents independently selected from halogen (e.g., F, Cl, Br, or I), OH, NH₂, NH(C₁-C₄) alkyl (e.g., methylamino, ethylamino, propylamino, or butylamino), N((C₁-C₄) alkyl)₂ (e.g., dimethylamino, diethylamino, dipropylamino, or dibutylamino), and heterocyclyl comprising one 5- to 7-membered ring and 1-3 heteroatoms selected from N, O, and S (e.g., pyrrolidinyl, pyrazolidinyl, imidazolidinyl, triazolidinyl, oxazolidinyl, isoxazolidinyl, oxadiazolidinyl, dioxazolidinyl, thiazolidinyl, isothiazolidinyl, thiadiazolidinyl, dithiazolidinyl, piperidinyl, hexahydropyridazinyl, hexahydropyrimidinyl, morpholinyl, dioxanyl, azepinyl, diazepinyl, etc.). In one embodiment, at least one R₁₃ is methyl, ethyl, or propyl. In one embodiment, at least one R₁₃ is ethyl, propyl, or butyl, wherein the ethyl, propyl, or butyl is optionally substituted with one to two substituents independently selected from NH₂, NH(C₁-C₄) alkyl(e.g., methylamino, ethylamino, propylamino, or butylamino), N((C₁-C₄) alkyl)₂ (e.g., dimethylamino, diethylamino, dipropylamino, or dibutylamino), and heterocycle comprising one 5- to 7-membered ring and 1-3 heteroatoms selected from N, O, and S. In one embodiment, at least one R₁₃ is ethyl, propyl, or butyl, wherein the ethyl, propyl, or butyl is optionally substituted with one to two substituents independently selected from N((C₁-C₄) alkyl)₂ (e.g., dimethylamino, diethylamino, dipropylamino, or dibutylamino) and heterocycle comprising one 5- to 7-membered ring and 1-3 heteroatoms selected from N, O, and S.

(XIII3) In one embodiment, at least one R₁₃ is C₃-C₇ cycloalkyl (e.g., cyclopropyl, cyclobutyl, cyclopentyl, cyclohexyl, or cycloheptyl).

(XIII4) In one embodiment, at least one R₁₃ is heterocyclyl comprising one 5- to 7-membered ring and 1-3 heteroatoms selected from N, O, and S (e.g., pyrrolidinyl, pyrazolidinyl, imidazolidinyl, triazolidinyl, oxazolidinyl, isoxazolidinyl, oxadiazolidinyl, dioxazolidinyl, thiazolidinyl, isothiazolidinyl, thiadiazolidinyl, dithiazolidinyl, piperidinyl, hexahydropyridazinyl, hexahydropyrimidinyl, morpholinyl, dioxanyl, azepinyl, diazepinyl, etc.) optionally substituted with one or more substituents independently selected from C₁-C₄ alkyl (e.g., methyl, ethyl, propyl, i-propyl, n-butyl, i-butyl, s-butyl, or t-butyl), halogen (e.g., F, Cl, Br, or I), OH, NH₂, NH(C₁-C₄) alkyl (e.g., methylamino, ethylamino, propylamino, or butylamino), N((C₁-C₄) alkyl)₂ (e.g., dimethylamino, diethylamino, dipropylamino, or dibutylamino), and heterocycle comprising one 5- to 7-membered ring and 1-3 heteroatoms selected from N, O, and S. In one embodiment, at least one R₁₃ is heterocycle comprising a 6-membered ring and 1-3 heteroatoms selected from N, O, and S, optionally substituted with one or more substituents independently selected from C₁-C₄ alkyl (e.g., methyl, ethyl, propyl, i-propyl, n-butyl, i-butyl, s-butyl, or t-butyl), halogen (e.g., F, Cl, Br, or I), OH, NH₂, NH(C₁-C₄) alkyl (e.g., methylamino, ethylamino, propylamino, or butylamino), N((C₁-C₄) alkyl)₂ (e.g., dimethylamino, diethylamino, dipropylamino, or dibutylamino), and heterocycle comprising one 5- to 7-membered ring and 1-3 heteroatoms selected from N, O, and S. In one embodiment, at least one R₁₃ is morpholinyl, piperidinyl, or piperazinyl, wherein the morpholinyl, piperidinyl, or piperazinyl is optionally substituted with one or more substituents independently selected from C₁-C₄ alkyl (e.g., methyl, ethyl, propyl, i-propyl, n-butyl, i-butyl, s-butyl, or t-butyl), halogen (e.g., F, Cl, Br, or I), OH, NH₂, NH(C₁-C₄) alkyl (e.g., methylamino, ethylamino, propylamino, or butylamino), N((C₁-C₄) alkyl)₂ (e.g., dimethylamino, diethylamino, dipropylamino, or dibutylamino), and heterocycle comprising one 5- to 7-membered ring and 1-3 heteroatoms selected from N, O, and S.

(XIV1) In one embodiment, at least one R₁₄ is H.

(XIV2) In one embodiment, at least one R₁₄ is C₁-C₃ alkyl (e.g., methyl, ethyl, propyl, or i-propyl).

(XV1) In one embodiment, m is 0.

(XV2) In one embodiment, in is 1.

(XVI1) In one embodiment, n is 0.

(XVI2) In one embodiment, n is 1.

(XVII1) In one embodiment, p is 0, 1, 2, or 3. In one embodiment, p is 0, 1, or 2.

(XVII2) In one embodiment, p is 0 or 1.

(XVI3) In one embodiment, p is 1 or 2. In one embodiment, p is 2 or 3.

(XVII4) In one embodiment, p is 0.

(XVII5) In one embodiment, p is 1.

(XVII6) In one embodiment, p is 2.

(XVII7) In one embodiment, p is 3.

(XVII8) In one embodiment, p is 4.

(XVIII1) In one embodiment, q is 0 or 1.

(XVIII2) In one embodiment, q is 1 or 2.

(XVIII3) In one embodiment, q is 0.

(XVII14) In one embodiment, q is 1.

(XIX1) In one embodiment, r is 0 or 1.

(XIX2) In one embodiment, r is 1 or 2.

(XIX3) In one embodiment, r is 0.

(XIX4) In one embodiment, r is 1.

In one embodiment, each of the substituents described for any one of X₁, R₁, R₂, R₃, R₄, R₅, R₆, R₇, R₈, R₉, R₁₁, R₁₂, R₁₃, R₁₄, m, n, p, q, and r can be combined with any of the substituents described for the remainder of X₁, R₁, R₂, R₃, R₄, R₅, R₆, R₇, R₈, R₉, R₁₁, R₁₂, R₁₃, R₁₄, m, n, p, q, and r.

For a compound of Formula I, II, or III, where applicable:

(1) In one embodiment, p is as described in (XVII6), and R₅ is as described in any one of (V3)-(V6).

(2) In one embodiment, X₁ is as described in (III2), and R₄ is as described in (IV1).

(3) In one embodiment, X₁ and R₄ are each as described in (2), and p and R₅ are each as described in (1).

(4) In one embodiment, X₁, R₄, R₅, and p, where applicable, are each as described in any one of (1)-(3), and m is as described in (XVI).

(5) In one embodiment, X₁, R₄, R₅, and p, where applicable, are each as described in any one of (1)-(3), and n is as described in (XVI1).

(6) In one embodiment, X₁, R₄, R₅, and p, where applicable, are each as described in any one of (1)-(3), m is as described in (XVI), and n is as described in (XVI1).

(7) In one embodiment, X₁, Ra, R₅, m, n, and p, where applicable, are each as described in any one of (1)-(6), Rh is as described in (VI4), and q is as described in (XVIII4).

(8) In one embodiment, X₁, R₄, R₅, R₆, m, n, p, and q, where applicable, are each as described in any one of (1)-(7), R₁ is as described in (X2).

In one embodiment, a compound of Formula I is Compound A

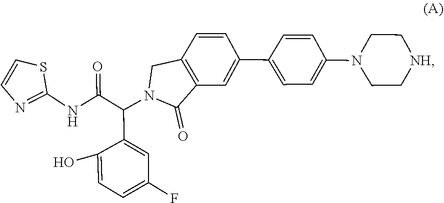 [SMILES: O=C(Nc1nccs1)C(c1cc(F)ccc1O)N1Cc2ccc(-c3ccc(N4CCNCC4)cc3)cc2C1=O]

or a pharmaceutically acceptable salt, hydrate, or solvate thereof.

In one embodiment, Compound A is a racemic mixture of the S-confromer and the R-conformer. In one embodiment, Compound A is racemnic mixture of less than 50% of the S-confromer and more than 50% of the R-conformer. In one embodiment, Compound A is racemic mixture of less than 40% of the S-confromer and more than 60% of the R-conformer. In one embodiment, Compound A is racemic mixture of less than 30% of the S-confromer and more than 70% of the R-conformer. In one embodiment, Compound A is racemic mixture of less than 20% of the S-confromer and more than 80% of the R-conformer. In one embodiment, Compound A is racemic mixture of less than 0% of the S-confromer and more than 90% of the R-conformer. In one embodiment, Compound A is racemic mixture of less than 5% of the S-confromer and more than 95% of the R-conformer. In one embodiment, Compound A is racemic mixture of less than 3% of the S-confromer and more than 97% of the R-conformer. In one embodiment, Compound A is racemic mixture of less than 1% of the S-confromer and more than 99% of the R-conformer. In one embodiment, Compound A is the R-conformer.

In one embodiment, Compound A is of the following structure:

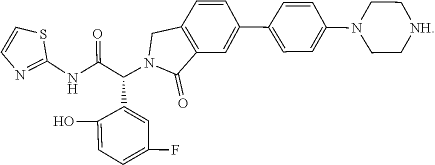 [SMILES: O=C(Nc1nccs1)[C@@H](c1cc(F)ccc1O)N1Cc2ccc(-c3ccc(N4CCNCC4)cc3)cc2C1=O]

In one embodiment, the ATP-competitive EGFR inhibitor is a compound of Formula I′:

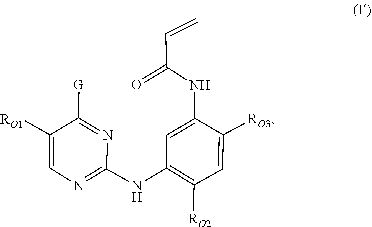

or a pharmaceutically acceptable salt, hydrate, or solvate thereof, wherein

G is 4,5,6,7-tetrahydropyrazolo[1,5-a]pyridin-3-yl, 1H-indol-3-yl, 1-methyl-1H-indol-3-yl, or pyrazolo[1,5-a]pyridin-3-yl;

R_{O1} is H, F, Cl, methyl, or CN;

R_{O2} is methoxy or methyl; and

R_{O3} is (3R)-3-(dimethylamino)pyrrolidin-1-yl, (3S)-3-(dimethylamino)pyrrolidin-1-yl, 3-(dimethylamino)azetidin-1-yl, (2-(dimethylamino)ethyl)-methylamino, (2-(methylamino)ethyl)-methylamino, 5-methyl-2,5-diazaspiro[3.4]oct-2-yl, (3aR,6aR)-5-methylhexahydro-pyrrolo[3,4-b]pyrrol-1(2H)-yl, 1-methyl-1,2,3,6-tetrahydropyridin-4-yl, 4-methylpiperizin-1-yl, 4-(2-(dimethylamino)-2-oxoethyl)piperazin-1-yl, methyl(2-(4-methylpiperazin-1-yl)ethyl)amino, methyl(2-(morpholin-4-yl)ethyl)amino, 1-amino-1,2,3,6-tetrahydropyridin-4-yl, or 4-((2S)-2-aminopropanoyl)piperazin-1-yl.

For a compound of Formula I′, where applicable, each of the variables can be a group as described below.

(i1) In one embodiment, G is 4,5,6,7-tetrahydropyrazolo[1,5-a]pyridin-3-yl.

(i2) In one embodiment, G is 1H-indol-3-yl.

(i3) In one embodiment, G is 1-methyl-H-indol-3-yl.

(4) In one embodiment, G is pyrazolo[1,5-a]pyridin-3-yl.

(ii1) In one embodiment, R_{O1} is H, F, Cl, or methyl.

(ii2) In one embodiment, R_{O1} is H.

(ii3) In one embodiment, R_{O1} is F or Cl.

(ii4) In one embodiment, R_{O1} is methyl.

(iii1) In one embodiment, R_{O2} is methoxy.

(iii2) In one embodiment, R_{O2} is methyl.

(iv1) In one embodiment, R_{O3} is (3R)-3-(dimethylamino)pyrrolidin-1-yl, (3S)-3-(dimethylamino)pyrrolidin-1-yl, 3-(dimethylamino)azetidin-1-yl, 5-methyl-2,5-diazaspiro[3.4]oct-2-yl, (3aR,6aR)-5-methylhexahydro-pyrrolo[3,4-b]pyrrol-1(2H)-yl, 1-methyl-1,2,3,6-tetrahydropyridin-4-yl, 4-methylpiperizin-1-yl, 4-(2-(dimethylamino)-2-oxoethyl)piperazin-1-yl, 1-amino-1,2,3,6-tetrahydropyridin-4-yl, or 4-((2S)-2-aminopropanoyl)piperazin-1-yl.

(iv2) In one embodiment, R_{O3} is (2-(dimethylamino)ethyl)-methylamino, (2-(methylamino)ethyl)-methylamino, methyl(2-(4-methylpiperazin-1-yl)ethyl)amino, or methyl(2-(morpholin-4-yl)ethyl)amino.

(iv3) In one embodiment, R_{O3} is (2-(dimethylamino)ethyl)-methylamino or (2-(methylamino)ethyl)-methylamino.

Any of the substituents described herein for any of G, R_{O1}, R_{O2}, and R_{O3} can be combined with any of the substituents described herein for one or more of the remainder of G, R_{O1}, R_{O2}, and R_{O3}.

In one embodiment, a compound of Formula I′ is of Formula I′a or I′b:

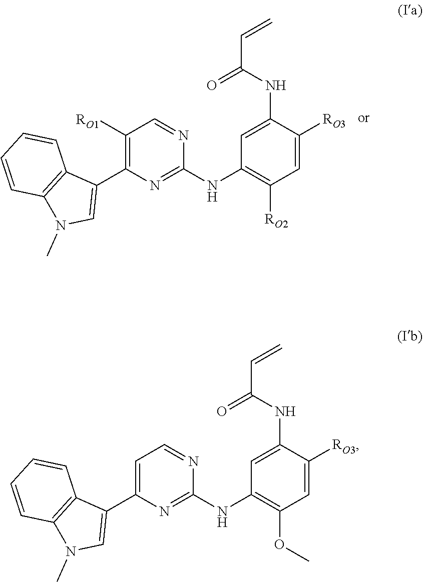

or a pharmaceutically acceptable salt, hydrate, or solvate thereof, wherein R_{O1}, R_{O2}, and R_{O3} are each as defined in Formula I′, and any of the substituents described herein for any of R_{O1}, R_{O2}, and R_{O3}, for example, in Formula I′, can be combined with any of the substituents described herein for one or more of the remainder of R_{O1}, R_{O2}, and R_{O3}, for example, in Formula I′.

In one embodiment, a compound of Formula I′ is Compound O

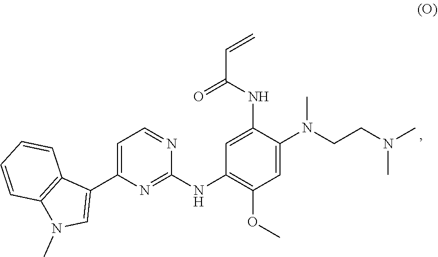 [SMILES: C=CC(=O)Nc1cc(Nc2nccc(-c3cn(C)c4ccccc34)n2)c(OC)cc1N(C)CCN(C)C]

or a pharmaceutically acceptable salt, hydrate, or solvate thereof.

In one embodiment, the application relates to a pharmaceutical combination comprising an allosteric EGFR inhibitor and an ATP-competitive EGFR inhibitor, wherein the allosteric EGFR inhibitor is Compound A or a pharmaceutically acceptable salt, hydrate, or solvate thereof, and the ATP-competitive EGFR inhibitor is Compound O or a pharmaceutically acceptable salt, hydrate, or solvate thereof.

The pharmaceutical combinations of the application are capable of modulating (e.g., inhibiting or decreasing) EGFR activity through binding to both an allosteric site in EGFR and a ATP-binding site in EGFR. In some embodiments, the pharmaceutical combinations of the application are capable of inhibiting or decreasing EGFR activity, without a second agent (e.g., an antibody such as cetuximab, trastuzumab, or panitumumab). In other embodiments, the pharmaceutical combinations of the present application, in combination with a second agent that prevents EGFR dimer formation (e.g., an antibody such as cetuximab, trastuzumab, or panitumumab), are capable of inhibiting or decreasing EGFR activity. In some embodiments, the second agent that prevents EGFR dimer formation is an antibody. In further embodiments, the second agent that prevents EGFR dimer formation is cetuximab, trastuzumab, or panitumumab. In further embodiments, the second agent that prevents EGFR dimer formation is cetuximab.

In some embodiments, the pharmaceutical combinations of the application are capable of modulating (e.g., inhibiting or decreasing) the activity of EGFR containing one or more mutations. In some embodiments, the mutant EGFR contains one or more mutations selected from T790M, L718Q, L844V, V948R, 858R, 1941R, C797S, Del (e.g., deletion in exon 19), and Insertion (e.g., insertion in exon 20). In some embodiments, the mutant EGFR contains C797S. In other embodiments, the mutant EGFR contains a combination of mutations, wherein the combination is selected from Del/L718Q, Del/L844V, Del/T790M, Del/T790M/L718Q, Del/T790M/L844V, L858R/L718Q, L858R/L844V, L858R/T790M, L858R/T790M/I941R, Del/T790M, Del/T790M/C797S, L858R/T790M/C797S, and L858R/T790M/L718Q. In other embodiments, the mutant EGFR contains a combination of mutations, wherein the combination is selected from Del/844V, L858R/L844V, L858R/T790M, L858R/T790M/I941R, L858R/T790M/C797S, Del/T790M, and Del/T790M/C797S. In other embodiments, the mutant EGFR contains a combination of mutations, wherein the combination is selected from L858RJT790M, L858R/T790M/I941R, L858R/T790M/C797S, Del/T790M, Del/T790M/C797S, and L858R/T790M.

In some embodiments, the pharmaceutical combinations of the present application, in combination with a second agent that prevents EGFR dimer formation, are capable of modulating (e.g., inhibiting or decreasing) the activity of EGFR containing one or more mutations (e.g., the EGFR containing one or more mutations described herein). In some embodiments, the second agent that prevents EGFR dimer formation is an antibody. In further embodiments, the second agent that prevents EGFR dimer formation is cetuximab, trastuzumab, or panitumumab. In further embodiments, the second agent that prevents EGFR dimer formation is cetuximab.

In some embodiments, the pharmaceutical combinations of the application are capable of modulating (e.g., inhibiting or decreasing) the activity of EGFR containing one or more mutations, but do not affect the activity of a wild-type EGFR.

In other embodiments, the pharmaceutical combinations of the present application, in combination with a second agent that prevents EGFR dimer formation, are capable of modulating (e.g., inhibiting or decreasing) the activity of EGFR containing one or more mutations, but do not affect the activity of a wild-type EGFR. In some embodiments, the second agent that prevents EGFR dimer formation is an antibody. In further embodiments, the second agent that prevents EGFR dimer formation is cetuximab, trastuzumab, or panitumumab. In further embodiments, the second agent that prevents EGFR dimer formation is cetuximab.

Modulation of EGFR containing one or more mutations, such as those described herein, but not a wild-type EGFR, provides a novel approach to the treatment, prevention, or amelioration of diseases including, but not limited to, cancer and metastasis, inflammation, arthritis, systemic lupus erthematosus, skin-related disorders, pulmonary disorders, cardiovascular disease, ischemia, neurodegenerative disorders, liver disease, gastrointestinal disorders, viral and bacterial infections, central nervous system disorders, Alzheimer's disease, Parkinson's disease, Huntington's disease, amyotrophic lateral sclerosis, spinal cord injury, and peripheral neuropathy.

In some embodiments, the pharmaceutical combinations of the application exhibit greater inhibition of EGFR containing one or more mutations as described herein relative to a wild-type EGFR. In certain embodiments, the pharmaceutical combinations of the application exhibit at least 2-fold, 3-fold, 5-fold, 10-fold, 25-fold, 50-fold or 100-fold greater inhibition of EGFR containing one or more mutations as described herein relative to a wild-type EGFR. In various embodiments, the pharmaceutical combinations of the application exhibit up to 1000-fold greater inhibition of EGFR containing one or more mutations as described herein relative to a wild-type EGFR. In various embodiments, the pharmaceutical combinations of the application exhibit up to 10000-fold greater inhibition of EGFR having a combination of mutations described herein relative to a wild-type EGFR.

In other embodiments, the pharmaceutical combinations of the application, in combination with a second agent that prevents EGFR dimer formation, exhibit greater inhibition of EGFR containing one or more mutations as described herein relative to a wild-type EGFR. In certain embodiments, the pharmaceutical combinations of the application, in combination with a second agent that prevents EGFR dimer formation, exhibit at least 2-fold, 3-fold, 5-fold, 10-fold, 25-fold, 50-fold or 100-fold greater inhibition of EGFR containing one or more mutations as described herein relative to a wild-type EGFR. In various embodiments, the pharmaceutical combinations of the application, in combination with a second agent that prevents EGFR dimer formation, exhibit up to 1000-fold greater inhibition of EGFR containing one or more mutations as described herein relative to a wild-type EGFR. In various embodiments, the pharmaceutical combinations of the application, in combination with a second agent that prevents EGFR dimer formation, exhibit up to 10000-fold greater inhibition of EGFR having a combination of mutations described herein relative to a wild-type EGFR. In some embodiments, the second agent that prevents EGFR dimer formation is an antibody. In further embodiments, the second agent that prevents EGFR dimer formation is cetuximab, trastuzumab, or panitumumab. In further embodiments, the second agent that prevents EGFR dimer formation is cetuximab.

In some embodiments, the pharmaceutical combinations of the application exhibit from about 2-fold to about 10-fold greater inhibition of EGFR containing one or more mutations as described herein relative to a wild-type EGFR. In various embodiments, the pharmaceutical combinations of the application exhibit from about 10-fold to about 100-fold greater inhibition of EGFR containing one or more mutations as described herein relative to a wild-type EGFR. In various embodiments, the pharmaceutical combinations of the application exhibit from about 100-fold to about 1000-fold greater inhibition of EGFR containing one or more mutations as described herein relative to a wild-type EGFR. In various embodiments, the pharmaceutical combinations of the application exhibit from about 1000-fold to about 10000-fold greater inhibition of EGFR containing one or more mutations as described herein relative to a wild-type EGFR.

In other embodiments, the pharmaceutical combinations of the application, in combination with a second agent that prevents EGFR dimer formation, exhibit from about 2-fold to about 10-fold greater inhibition of EGFR containing one or more mutations as described herein relative to a wild-type EGFR. In other embodiments, the pharmaceutical combinations of the application, in combination with a second agent that prevents EGFR dimer formation, exhibit from about 10-fold to about 100-fold greater inhibition of EGFR containing one or more mutations as described herein relative to a wild-type EGFR. In other embodiments, the pharmaceutical combinations of the application, in combination with a second agent that prevents EGFR dimer formation, exhibit from about 100-fold to about 1000-fold greater inhibition of EGFR containing one or more mutations as described herein relative to a wild-type EGFR. In other embodiments, the pharmaceutical combinations of the application, in combination with a second agent that prevents EGFR dimer formation, exhibit from about 1000-fold to about 10000-fold greater inhibition of EGFR containing one or more mutations as described herein relative to a wild-type EGFR. In some embodiments, the second agent that prevents EGFR dimer formation is an antibody. In further embodiments, the second agent that prevents EGFR dimer formation is cetuximab, trastuzumab, or panitumumab. In further embodiments, the second agent that prevents EGFR dimer formation is cetuximab.

In some embodiments, the inhibition of EGFR activity is measured by IC₅₀.

In some embodiments, the inhibition of EGFR activity is measured by EC₅₀.

The allosteric EGFR inhibitors of the pharmaceutical combinations of the application bind to an allosteric site in EGFR. In some embodiments, the allosteric EGFR inhibitors interact with at least one amino acid residue of EGFR selected from Lys745, Leu788, and Ala 743. In other embodiments, the allosteric EGFR inhibitors interact with at least one amino acid residue of EGFR selected from Cys755, Leu777, Phe856, and Asp855. In other embodiments, the allosteric EGFR inhibitors interact with at least one amino acid residue of EGFR selected from Met766, Ie759, Glu762, and Ala763. In other embodiments, the allosteric EGFR inhibitors interact with at least one amino acid residue of EGFR selected from Lys745, Leu788, and Ala 743, at least one amino acid residue of EGFR selected from Cys755, Leu777, Phe856, and Asp855, and at least one amino acid residue of EGFR selected from Met766, Ile759, Glu762, and Ala763. In other embodiments, the allosteric EGFR inhibitors do not interact with the any of the amino acid residues of EGFR selected from Met793, Gly796, and Cys797.

The ATP-competitive EGFR inhibitors of the pharmaceutical combinations of the application bind to an ATP-binding site in EGFR.

In some embodiments, the pharmaceutical combinations of the application can be at least about 2-fold, 3-fold, 5-fold, 10-fold, 25-fold, 50-fold or about 100-fold more potent at inhibiting the kinase activity of a drug-resistant EGFR mutant relative to a wild-type EGFR. In some embodiments, the drug-resistant EGFR mutant is resistant to one or more known EGFR inhibitors, including but not limited to gefitinib, erlotinib, afatinib, lapatinib, neratinib,

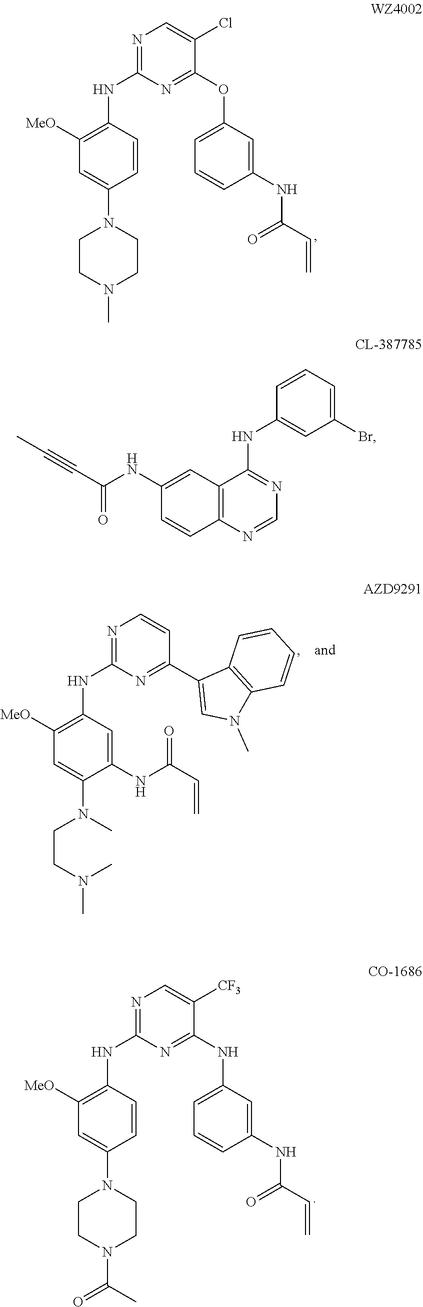

In some embodiments, the drug-resistant EGFR mutant comprises a sensitizing mutation, such as Del and L858R.

In some embodiments, the pharmaceutical combinations of the application, in combination with a second agent that prevents EGFR dimer formation, can be at least about 2-fold, 3-fold, 5-fold, 10-fold, 25-fold, 50-fold or about 100-fold more potent at inhibiting the kinase activity of a drug-resistant EGFR mutant relative to a wild-type EGFR. In some embodiments, the drug-resistant EGFR mutant is resistant to one or more known EGFR inhibitors, including but not limited to gefitinib, erlotinib, afatinib, lapatinib, neratinib, WZ4002, CL-387785, AZD9291, and CO-1686. In some embodiments, the drug-resistant EGFR mutant comprises a sensitizing mutation, such as Del and L858R. In some embodiments, the second agent that prevents EGFR dimer formation is an antibody. In further embodiments, the second agent that prevents EGFR dimer formation is cetuximab, trastuzumab, or panitumumab. In further embodiments, the second agent that prevents EGFR dimer formation is cetuximab.

In some embodiments, the pharmaceutical combinations of the application inhibit kinase activity of a drug-resistant EGFR mutant harboring a sensitizing mutation (e.g., Del and L858R) and a drug-resistance mutation (e.g., T790M, L718Q, C797S, and L844V) with less than a 10-fold difference in potency (e.g., as measured by IC₅₀) relative to an EGFR mutant harboring the sensitizing mutation but not the drug-resistance mutation. In some embodiments, the difference in potency is less than about 9-fold, 8-fold, 7-fold, 6-fold, 5-fold, 4-fold, 3-fold, or 2-fold.

In other embodiments, the pharmaceutical combinations of the application, in combination with a second agent that prevents EGFR dimer formation, inhibit kinase activity of a drug-resistant EGFR mutant harboring a sensitizing mutation (e.g., Del and L858R) and a drug-resistance mutation (e.g., T790M, L718Q, C797S, and L844V) with less than a 10-fold difference in potency (e.g., as measured by IC₅₀) relative to an EGFR mutant harboring the sensitizing mutation but not the drug-resistance mutation. In some embodiments, the difference in potency is less than about 9-fold, 8-fold, 7-fold, 6-fold, 5-fold, 4-fold, 3-fold, or 2-fold. In some embodiments, the second agent that prevents EGFR dimer formation is an antibody. In further embodiments, the second agent that prevents EGFR dimer formation is cetuximab, trastuzumab, or panitumumab. In further embodiments, the second agent that prevents EGFR dimer formation is cetuximab.

In some embodiments, the pharmaceutical combinations of the application are more potent than one or more known EGFR inhibitors, including but not limited to gefitinib, erlotinib, afatinib, lapatinib, neratinib, WZ4002, CL-387785, AZD9291, and CO-1686, at inhibiting the activity of EGFR containing one or more mutations as described herein, for example, at least about 2-fold, 3-fold, 5-fold, 10-fold, 25-fold, 50-fold or about 100-fold more potent (e.g., as measured by IC₅₀) than gefitinib, erlotinib, afatinib, lapatinib, neratinib, WZ4002, CL-387785, AZD9291, and CO-1686.

In other embodiments, the pharmaceutical combinations of the application, in combination with a second agent that prevents EGFR dimer formation, are more potent than one or more known EGFR inhibitors, including but not limited to gefitinib, erlotinib, afatinib, lapatinib, neratinib, WZ4002, CL-387785, AZD9291, and CO-1686, at inhibiting the activity of EGFR containing one or more mutations as described herein, for example, at least about 2-fold, 3-fold, 5-fold, 10-fold, 25-fold, 50-fold or about 100-fold more potent (e.g., as measured by IC₅₀) than gefitinib, erlotinib, afatinib, lapatinib, neratinib, WZ4002, CL-387785, AZD9291, and CO-1686. In some embodiments, the second agent that prevents EGFR dimer formation is an antibody. In further embodiments, the second agent that prevents EGFR dimer formation is cetuximab, trastuzumab, or panitumumab. In further embodiments, the second agent that prevents EGFR dimer formation is cetuximab.

In some embodiments, the pharmaceutical combinations of the application are less potent than one or more known EGFR inhibitors, including but not limited to gefitinib, erlotinib, afatinib, lapatinib, neratinib, WZ4002, CL-387785, AZD9291, and CO-1686, at inhibiting the activity of a wild-type EGFR, for example, at least about 2-fold, 3-fold, 5-fold, 10-fold, 25-fold, 50-fold or about 100-fold less potent (e.g., as measured by IC₅₀) than gefitinib, erlotinib, afatinib, lapatinib, neratinib, WZ4002, CL-387785, AZD9291, and CO-1686.

In other embodiments, the pharmaceutical combinations of the application, in combination with a second agent that prevents EGFR dimer formation, are less potent than one or more known EGFR inhibitors, including but not limited to gefitinib, erlotinib, afatinib, lapatinib, neratinib, WZ4002, CL-387785, AZD9291, and CO-1686, at inhibiting the activity of a wild-type EGFR, for example, at least about 2-fold, 3-fold, 5-fold, 10-fold, 25-fold, 50-fold or about 100-fold less potent (e.g., as measured by IC₅₀) than gefitinib, erlotinib, afatinib, lapatinib, neratinib, WZ4002, CL-387785, AZD9291, and CO-1686. In some embodiments, the second agent that prevents EGFR dimer formation is an antibody. In further embodiments, the second agent that prevents EGFR dimer formation is cetuximab, trastuzumab, or panitumumab. In further embodiments, the second agent that prevents EGFR dimer formation is cetuximab.

Potency of the inhibitor can be determined by EC₅₀ value. An agent with a lower EC₅₀ value, as determined under substantially similar conditions, is a more potent inhibitor relative to an agent with a higher EC₅₀ value. In some embodiments, the substantially similar conditions comprise determining an EGFR-dependent phosphorylation level, in vitro or in vivo (e.g., in 3T3 cells expressing a wild type EGFR, a mutant EGFR, or a fragment of any thereof).

Potency of the inhibitor can also be determined by IC₅₀ value. An agent with a lower IC₅₀ value, as determined under substantially similar conditions, is a more potent inhibitor relative to an agent with a higher IC₅₀ value. In some embodiments, the substantially similar conditions comprise determining an EGFR-dependent phosphorylation level, in vitro or in vivo (e.g., in 3T3 cells expressing a wild type EGFR, a mutant EGFR, or a fragment of any thereof).

An EGFR sensitizing mutation comprises without limitation L858R, G719S, G719C, G719A, L861Q, a deletion in exon 19 and/or an insertion in exon 20. A drug-resistant EGFR mutant can have without limitation a drug resistance mutation comprising T790M, T854A, L718Q, C797S, or D761Y.

The selectivity between wild-type EGFR and EGFR containing one or more mutations as described herein can be measured using cellular proliferation assays where cell proliferation is dependent on kinase activity. For example, murine Ba/F3 cells transfected with a suitable version of wild-type EGFR (such as VIII; containing a WT EGFR kinase domain), or Ba/F3 cells transfected with L858R/T790M, Del/T790ML718Q, L858R/T790M/L718Q, L858R/T790M/C797S, Del/T790M/C797S, L858R/T790M/I941R, or Exon 19 deletion/T790M can be used. Proliferation assays are performed at a range of inhibitor concentrations (e.g., 10 μM, 3 μM, 1 μM, 330 nM, 110 nM, 33 nM, 11 nM, 3 nM, 1 nM) and an EC₅₀ is calculated.

An alternative method to measure effects on EGFR activity is to assay EGFR phosphorylation. Wild type or mutant (L858R/T790M, Del/T790M, Del/T790M/L718Q, L858R/T790M/C797S, Del/T790M/C797S, L858R/T790M/I941R, or L858R/T790M/L718Q) EGFR can be transfected into cells which do not normally express endogenous EGFR and the ability of the inhibitor (using concentrations as above) to inhibit EGFR phosphorylation can be assayed. Cells are exposed to increasing concentrations of inhibitor and stimulated with EGF. The effects on EGFR phosphorylation are assayed by Western Blotting using phospho-specific EGFR antibodies.

In some embodiments, the pharmaceutical combinations of the application exhibit greater than 2-fold, 3-fold, 5-fold, 10-fold, 25-fold, 50-fold, 100-fold, or 1000-fold inhibition of EGFR containing one or more mutations as described herein (e.g., L858R/T790M, Del/T790M, Del/T790M/L718Q, L858R/T790M/C797S, Del/T790M/C797S, L858R/T790M/I941R, or L858R/T790M/L718Q) relative to a wild-type EGFR.

In other embodiments, the pharmaceutical combinations of the application, in combination with a second agent that prevents EGFR dimer formation, exhibit greater than 2-fold, 3-fold, 5-fold, 10-fold, 25-fold, 50-fold, 100-fold, or 1000-fold inhibition of EGFR containing one or more mutations as described herein (e.g., L858R/T790M, Del/T790M, Del/T790M/I718Q, Del/T790M/C797S, L858R/T790M/C797S, L858R/T790M/I941R, or L858R/T790M/L718Q) relative to a wild-type EGFR. In some embodiments, the second agent that prevents EGFR dimer formation is an antibody. In further embodiments, the second agent that prevents EGFR dimer formation is cetuximab, trastuzumab, or panitumumab. In further embodiments, the second agent that prevents EGFR dimer formation is cetuximab.

In another aspect, the application provides a kit comprising an allosteric EGFR inhibitor, as described herein, and an ATP-competitive EGFR inhibitor. In some embodiments, the kit comprises instructions for its administration. In certain embodiments, the kit further comprises components for performing a test to determine whether a subject has activating and/or drug resistance mutations in EGFR. In some embodiments, the kit further comprises a second agent. In some embodiments, the second agent that prevents EGFR dimer formation is an antibody. In further embodiments, the second agent that prevents EGFR dimer formation is cetuximab, trastuzumab, or panitumumab. In further embodiments, the second agent that prevents EGFR dimer formation is cetuximab.

Another aspect is an isotopically labeled compound of any of the formulae delineated herein. Such compounds have one or more isotope atoms which may or may not be radioactive (e.g., ³H, ²H, ¹⁴C, ¹³C, ¹⁸F, ³⁵S, ³²P, ¹²⁵I, and ¹³¹I) introduced into the compound. Such compounds are useful for drug metabolism studies and diagnostics, as well as therapeutic applications.

The compounds of the application are defined herein by their chemical structures and/or chemical names. Where a compound is referred to by both a chemical structure and a chemical name, and the chemical structure and chemical name conflict, the chemical structure is determinative of the compound's identity.

The recitation of a listing of chemical groups in any definition of a variable herein includes definitions of that variable as any single group or combination of listed groups. The recitation of an embodiment for a variable herein includes that embodiment as any single embodiment or in combination with any other embodiments or portions thereof.

### Definitions

Listed below are definitions of various terms used to describe this application. These definitions apply to the terms as they are used throughout this specification and claims, unless otherwise limited in specific instances, either individually or as part of a larger group.

The term “alkyl,” as used herein, refers to saturated, straight- or branched-chain hydrocarbon radicals containing, in certain embodiments, between one and six, or one and eight carbon atoms, respectively. Examples of C₁-C₆ alkyl radicals include, but are not limited to, methyl, ethyl, propyl, isopropyl, n-butyl, tert-butyl, neopentyl, n-hexyl radicals; and examples of C₁-C₈ alkyl radicals include, but are not limited to, methyl, ethyl, propyl, isopropyl, n-butyl, tert-butyl, neopentyl, n-hexyl, heptyl, octyl radicals.

The term “alkenyl,” as used herein, denotes a monovalent group derived from a hydrocarbon moiety containing, in certain embodiments, from two to six, or two to eight carbon atoms having at least one carbon-carbon double bond. The double bond may or may not be the point of attachment to another group. Alkenyl groups include, but are not limited to, for example, ethenyl, propenyl, butenyl, 1-methyl-2-buten-1-yl, heptenyl, octenyl and the like.

The term “alkynyl,” as used herein, denotes a monovalent group derived from a hydrocarbon moiety containing, in certain embodiments, from two to six, or two to eight carbon atoms having at least one carbon-carbon triple bond. The alkynyl group may or may not be the point of attachment to another group. Representative alkynyl groups include, but are not limited to, for example, ethynyl, 1-propynyl, 1-butynyl, heptynyl, octynyl and the like.

The term “alkoxy” refers to an —O-alkyl radical.

The term “aryl,” as used herein, refers to a mono- or poly-cyclic carbocyclic ring system having one or more aromatic rings, fused or non-fused, including, but not limited to, phenyl, naphthyl, tetrahydronaphthyl, indanyl, indenyl and the like.

The term “aralkyl,” as used herein, refers to an alkyl residue attached to an aryl ring. Examples include, but are not limited to, benzyl, phenethyl and the like.

The term “cycloalkyl,” as used herein, denotes a monovalent group derived from a monocyclic or polycyclic saturated or partially unsaturated carbocyclic ring compound. Examples of C₃-C₈ cycloalkyl include, but not limited to, cyclopropyl, cyclobutyl, cyclopentyl, cyclohexyl, cyclopentyl and cyclooctyl; and examples of C₃-C₁₂-cycloalkyl include, but not limited to, cyclopropyl, cyclobutyl, cyclopentyl, cyclohexyl, bicyclo [2.2.1] heptyl, and bicyclo [2.2.2] octyl. Also contemplated is a monovalent group derived from a monocyclic or polycyclic carbocyclic ring compound having at least one carbon-carbon double bond by the removal of a single hydrogen atom. Examples of such groups include, but are not limited to, cyclopropenyl, cyclobutenyl, cyclopentenyl, cyclohexenyl, cycloheptenyl, cyclooctenyl, and the like.

The term “heteroaryl,” as used herein, refers to a mono- or poly-cyclic (e.g., bi-, or tri-cyclic or more) fused or non-fused, radical or ring system having at least one aromatic ring, having from five to ten ring atoms of which one ring atoms is selected from S, O, and N; zero, one, or two ring atoms are additional heteroatoms independently selected from S, O, and N; and the remaining ring atoms are carbon. Heteroaryl includes, but is not limited to, pyridinyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, benzimidazolyl, benzooxazolyl, quinoxalinyl, and the like.

The term “heteroaralkyl,” as used herein, refers to an alkyl residue attached to a heteroaryl ring. Examples include, but are not limited to, pyridinylmethyl, pyrimidinylmethyl and the like.

The term “heterocyclyl,” or “heterocycloalkyl,” as used herein, refers to a non-aromatic 3-, 4-, 5-, 6- or 7-membered ring or a bi- or tri-cyclic group fused of non-fused system, where (i) each ring contains between one and three heteroatoms independently selected from oxygen, sulfur and nitrogen, (ii) each 5-membered ring has 0 to 1 double bonds and each 6-membered ring has 0 to 2 double bonds, (iii) the nitrogen and sulfur heteroatoms may optionally be oxidized, and (iv) the nitrogen heteroatom may optionally be quaternized. Representative heterocycloalkyl groups include, but are not limited to, [1,3]dioxolane, pyrrolidinyl, pyrazolinyl, pyrazolidinyl, imidazolinyl, imidazolidinyl, piperidinyl, piperazinyl, oxazolidinyl, isoxazolidinyl, morpholinyl, thiazolidinyl, isothiazolidinyl, and tetrahydrofuryl.

The term “alkylamino” refers to a group having the structure —NH(C₁-C₁₂ alkyl), e.g., —NH(C₁-C₆ alkyl), where C₁-C₁₂ alkyl is as previously defined.

The term “dialkylamino” refers to a group having the structure —N(C₁-C₁₂ alkyl)₂, e.g., —NH(C₁-C₆ alkyl), where C₁-C₁₂ alkyl is as previously defined.

The term “acyl” includes residues derived from acids, including but not limited to carboxylic acids, carbamic acids, carbonic acids, sulfonic acids, and phosphorous acids. Examples include aliphatic carbonyls, aromatic carbonyls, aliphatic sulfonyls, aromatic sulfinyls, aliphatic sulfinyls, aromatic phosphates and aliphatic phosphates. Examples of aliphatic carbonyls include, but are not limited to, acetyl, propionyl, 2-fluoroacetyl, butyryl, 2-hydroxy acetyl, and the like.

In accordance with the application, any of the aryls, substituted aryls, heteroaryls and substituted heteroaryls described herein, can be any aromatic group. Aromatic groups can be substituted or unsubstituted.

The terms “hal,” “halo,” and “halogen,” as used herein, refer to an atom selected from fluorine, chlorine, bromine and iodine.

As described herein, compounds of the application may optionally be substituted with one or more substituents, such as are illustrated generally above, or as exemplified by particular classes, subclasses, and species of the application. It will be appreciated that the phrase “optionally substituted” is used interchangeably with the phrase “substituted or unsubstituted.” In general, the term “substituted”, whether preceded by the term “optionally” or not, refers to the replacement of hydrogen radicals in a given structure with the radical of a specified substituent. Unless otherwise indicated, an optionally substituted group may have a substituent at each substitutable position of the group, and when more than one position in any given structure may be substituted with more than one substituent selected from a specified group, the substituent may be either the same or different at every position. The terms “optionally substituted”, “optionally substituted alkyl,” “optionally substituted “optionally substituted alkenyl,” optionally substituted alkynyl”, “optionally substituted cycloalkyl,” “optionally substituted cycloalkenyl,” “optionally substituted aryl”, “optionally substituted heteroaryl,” “optionally substituted aralkyl”, “optionally substituted heteroaralkyl,” “optionally substituted heterocycloalkyl,” and any other optionally substituted group as used herein, refer to groups that are substituted or unsubstituted by independent replacement of one, two, or three or more of the hydrogen atoms thereon with substituents including, but not limited to:

—F, —CI, —Br, —I, —OH, protected hydroxy, —NO₂, —CN, —NH₂, protected amino, —NH—C₁-C₁₂-alkyl, —NH—C₂-C₁₂-alkenyl, —NH—C₂-C₁₂-alkenyl, —NH—C₃-C₁₂-cycloalkyl, —NH-aryl, —NH-heteroaryl, —NH-heterocycloalkyl, -dialkylamino, -diarylamino, -diheteroarylamino, —O—C₁-C₁₂-alkyl, —O—C₂-C₁₂-alkenyl, —O—C₂-C₁₂-alkenyl, —O—C3-C₁₂-cycloalkyl, —O-aryl, —O-heteroaryl, —O-heterocycloalkyl, —C(O)—C₁-C₁₂-alkyl, —C(O)—C₂-C₁₂-alkenyl, —C(O)—C₂-C₁₂-alkenyl, —C(O)—C₃-C₁₂-cycloalkyl, —C(O)-aryl, —C(O)-heteroaryl, —C(O)-heterocycloalkyl, —CONH₂, —CONH—C₁-C₁₂-alkyl, —CONH—C₂-C₁₂-alkenyl, —CONH—C₂-C₁₂-alkenyl, —CONH—C₃-C₂-cycloalkyl, —CONH-aryl, —CONH-heteroaryl, —CONH-heterocycloalkyl, —O—OCO₂—C₁-C₁₂-alkyl, —OCO₂—C₂-C₁₂-alkenyl, —CO₂—C₂-C₁₂-alkenyl, —OCO₂—C₃-C₁₂-cycloalkyl, —OCO₂-aryl, —OCO₂-heteroaryl, —OCO₂-heterocycloalkyl, —OCONH₂, —OCONH—C₁-C₁₂-alkyl, —OCONH—C₂-C₁₂-alkenyl, —OCONH—C₂-C₁₂-alkenyl, —OCONH—C₃-C₁₂-cycloalkyl, —OCONH-aryl, —OCONH-heteroaryl, —OCONH-heterocycloalkyl, —NHC(O)—C₁-C₁₂-alkyl, —NHC(O)—C₂-C₁₂-alkenyl, —NHC(O)—C₂-C₁₂-alkenyl, —NHC(O)—C₃-C₁₂-cycloalkyl, —NHC(O)-aryl, —NHC(O)-heteroaryl, —NHC(O)-heterocycloalkyl, —NHCO₂—C₁-C₁₂-alkyl, —NHCO₂—C₂-C₁₂-alkenyl, —NHCO₂—C₂-C₁₂-alkenyl, —NHCO₂—C₃-C₁₂-cycloalkyl, —NHCO₂-aryl, —NHCO₂-heteroaryl, —NHCO₂-heterocycloalkyl, NHC(O)NH₂, —NHC(O)NH—C₁-C₁₂-alkyl, —NHC(O)NH—C₂-C₂-alkenyl, —NHC(O)NH—C₂-C₁₂-alkenyl, —NHC(O)NH—C₃-C₁₂-cycloalkyl, —NHC(O)NH-aryl, —NHC(O)NH-heteroaryl, NHC(O)NH-heterocycloalkyl, —NHC(S)NH₂, —NHC(S)NH—C₁-C₁₂-alkyl, —NHC(S)NH—C₂-C₁₂-alkenyl, —NHC(S)NH—C₂-C₁₂-alkenyl, —NHC(S)NH—C₃-C₁₂-cycloalkyl, —NHC(S)NH-aryl, —NHC(S)NH-heteroaryl, —NHC(S)NH-heterocycloalkyl, —NHC(NH)NH₂, —NHC(NH)NH—C₁-C₁₂-alkyl, —NHC(NH)NH—C₂-C₁₂-alkenyl, —NHC(NH)NH—C₂-C₁₂-alkenyl, —NHC(NH)NH—C₃-C₁₂-cycloalkyl, —NH C(NH)NH-aryl, —NHC(NH)NH-heteroaryl, —NHC(NH)NHheterocycloalkyl, —NHC(NH)—C₁-C₁₂-alkyl, —NHC(NH)—C₂-C₁₂-alkenyl, —NHC(NH)—C₂-C₁₂-alkenyl, —NHC(NH)—C₃-C₁₂-cycloalkyl, —NHC(NH)-aryl, —NHC(NH)-heteroaryl, —NHC(NH)-heterocycloalkyl, —C(NH)NH—C₁-C₁₂-alkyl, —C(NH)NH—C₂-C₁₂-alkenyl, —C(NH)NH—C₂-C₁₂-alkenyl, C(NH)NH—C₃-C₁₂-cycloalkyl, —C(NH)NH-aryl, —C(NH)NH-heteroaryl, —C(NH)NHheterocycloalkyl, —S(O)—C₁-C₁₂-alkyl, —S(O)—C₂-C₁₂-alkenyl, —S(O)—C₂-C₁₂-alkenyl, —S(O)—C₃-C₁₂-cycloalkyl, —S(O)-aryl, —S(O)-heteroaryl, —S(O)-heterocycloalkyl —SO₂NH₂, —SO₂NH—C₁-C₁₂-alkyl, —SO₂NH—C₂-C₁₂-alkenyl, —SO₂NH—C₂-C₁₂-alkenyl, —SO₂NH—C₃-C₁₂-cycloalkyl, —SO₂NH-aryl, —SO₂NH-heteroaryl, —SO₂NH-heterocycloalkyl, —NHSO₂—C₁-C₁₂-alkyl, —NHSO₂—C₂-C₁₂-alkenyl, —NHSO₂—C₂-C₁₂-alkenyl, —NHSO₂—C₃-C₁₂-cycloalkyl, —NHSO₂-aryl, —NHSO₂-heteroaryl, —NHSO₂-heterocycloalkyl, —CH₂NH₂, —CH₂SO₂CH₃, -aryl, -arylalkyl, -heteroaryl, -heteroarylalkyl, -heterocycloalkyl, —C₃-C₁₂-cycloalkyl, polyalkoxyalkyl, polyalkoxy, -methoxymethoxy, -methoxyethoxy, —SH, —S—C₁-C₁₂-alkyl, —S—C₂-C₁₂-alkenyl, —S—C₂-C₁₂-alkenyl, —S—C₃-C₁₂-cycloalkyl, —S-aryl, —S-heteroaryl, —S-heterocycloalkyl, or methylthiomethyl.

It is understood that the aryls, heteroaryls, alkyls, and the like can be substituted.

The term “cancer” includes, but is not limited to, the following cancers: epidermoid Oral: buccal cavity, lip, tongue, mouth, pharynx; Cardiac: sarcoma (angiosarcoma, fibrosarcoma, rhabdomyosarcoma, liposarcoma), myxoma, rhabdomyoma, fibroma, lipoma, and teratoma; Lung: bronchogenic carcinoma (squamous cell or epidermoid, undifferentiated small cell, undifferentiated large cell, adenocarcinoma), alveolar (bronchiolar) carcinoma, bronchial adenorna, sarcoma, lymphoma, chondromatous hamartoma, mesothelioma; Gastrointestinal: esophagus (squamous cell carcinoma, larynx, adenocarcinoma, leiomyosarcoma, lymphoma), stomach (carcinoma, lymphoma, leiomyosarcoma), pancreas (ductal adenocarcinoma, insulinoma, glucagonoma, gastrinoma, carcinoid tumors, vipoma), small bowel or small intestines (adenocarcinoma, lymphoma, carcinoid tumors, Karposi's sarcoma, leiomyoma, hemangioma, lipoma, neurofibroma, fibroma), large bowel or large intestines (adenocarcinoma, tubular adenoma, villous adenoma, hamartoma, leiomyoma), colon, colon-rectum, colorectal, rectum; Genitourinary tract: kidney (adenocarcinoma, Wilm's tumor (nephroblastoma), lymphoma, leukemia), bladder and urethra (squamous cell carcinoma, transitional cell carcinoma, adenocarcinoma), prostate (adenocarcinoma, sarcoma), testis (seminoma, teratoma, embryonal carcinoma, teratocarcinoma, choriocarcinoma, sarcoma, interstitial cell carcinoma, fibroma, fibroadenoma, adenomatoid tumors, lipoma); Liver: hepatoma (hepatocellular carcinoma), cholangiocarcinoma, hepatoblastoma, angiosarcoma, hepatocellular adenoma, hemangioma, biliary passages; Bone: osteogenic sarcoma (osteosarcoma), fibrosarcoma, malignant fibrous histiocytoma, chondrosarcoma, Ewing's sarcoma, malignant lymphoma (reticulum cell sarcoma), multiple myeloma, malignant giant cell tumor chordoma, osteochronfroma (osteocartilaginous exostoses), benign chondroma, chondroblastoma, chondromyxofibroma, osteoid osteoma and giant cell tumors; Nervous system: skull (osteoma, hemangioma, granuloma, xanthoma, osteitis deformans), meninges (meningioma, meningiosarcoma, gliomatosis), brain (astrocytoma, medulloblastoma, glioma, ependymoma, germinoma (pinealoma), glioblastoma multiform, oligodendroglioma, schwannoma, retinoblastoma, congenital tumors), spinal cord neurofibroma, meningioma, glioma, sarcoma); Gynecological: uterus (endometrial carcinoma), cervix (cervical carcinoma, pre-tumor cervical dysplasia), ovaries (ovarian carcinoma (serous cystadenocarcinoma, mucinous cystadenocarcinoma, unclassified carcinoma), granulosa-thecal cell tumors, Sertoli-Leydig cell tumors, dysgerminoma, malignant teratoma), vulva (squamous cell carcinoma, intraepithelial carcinoma, adenocarcinoma, fibrosarcoma, melanoma), vagina (clear cell carcinoma, squamous cell carcinoma, botryoid sarcoma (embryonal rhabdomyosarcoma), fallopian tubes (carcinoma), breast; Hematologic: blood (myeloid leukemia (acute and chronic), acute lymphoblastic leukemia, chronic lymphocytic leukemia, myeloproliferative diseases, multiple myeloma, myelodysplastic syndrome), Hodgkin's disease, non-Hodgkin's lymphoma (malignant lymphoma) hairy cell; lymphoid disorders; Skin: malignant melanoma, basal cell carcinoma, squamous cell carcinoma, Karposi's sarcoma, keratoacanthoma, moles dysplastic nevi, lipoma, angioma, dermatofibroma, keloids, psoriasis, Thyroid gland: papillary thyroid carcinoma, follicular thyroid carcinoma; medullary thyroid carcinoma, undifferentiated thyroid cancer, multiple endocrine neoplasia type 2A, multiple endocrine neoplasia type 2B, familial medullary thyroid cancer, pheochromocytoma, paraganglioma; and Adrenal glands: neuroblastoma. Thus, the term “cancerous cell” as provided herein, includes a cell afflicted by any one of the above-identified conditions.

The term “EGFR” herein refers to epidermal growth factor receptor kinase.

The term “HER” or “Her”, herein refers to human epidermal growth factor receptor kinase.

The term “subject” as used herein refers to a mammal. A subject therefore refers to, for example, dogs, cats, horses, cows, pigs, guinea pigs, and the like. Preferably the subject is a human. When the subject is a human, the subject may be referred to herein as a patient.

“Treat”, “treating” and “treatment” refer to a method of alleviating or abating a disease and/or its attendant symptoms.

As used herein, “preventing” or “prevent” describes reducing or eliminating the onset of the symptoms or complications of the disease, condition or disorder.

As used herein, the term “allosteric site” refers to a site on EGFR other than the ATP binding site, such as that characterized in a crystal structure of EGFR. An “allosteric site” can be a site that is close to the ATP binding site, such as that characterized in a crystal structure of EGFR_. For example, one allosteric site includes one or more of the following amino acid residues of EGFR: Lys745, Leu788, Ala 743, Cys755, Leu777, Phe856, Asp855, Met766, Ile759, Glu762, and/or Ala763.

As used herein, the term “allosteric EGFR inhibitor” refers to a compound that inhibits EGFR activity through binding to one or more allosteric sites on EGFR.

As used herein, the term “ATP-competitive EGFR inhibitor” refers to a compound that inhibits EGFR activity through binding to one or more ATP-binding sites on EGFR.

As used herein, the term “agent that prevents EGFR dimer formation” refers to an agent that prevents dimer formation in which the C-lobe of the “activator” subunit impinges on the N-lobe of the “receiver” subunit. Examples of agents that prevent EGFR dimer formation include, but are not limited to, cetuximab, cobimetinib, trastuzumab, panitumumab, and Mig6.

As used herein the term “GDC0973” or “Cobimetinib” refers to a compound having the chemical structure:

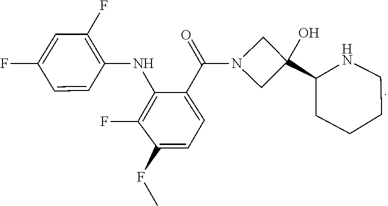

As used herein, the term “pharmaceutically acceptable salt” refers to those salts of a compound formed by the process of the present application which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, S. M. Berge, et al., describes pharmaceutically acceptable salts in detail in J. Pharmaceutical Sciences, 66: 1-19 (1977). The salts can be prepared in situ during the final isolation and purification of the compounds of the application, or separately by reacting the free base function with a suitable organic acid.

Examples of pharmaceutically acceptable include, but are not limited to, nontoxic acid addition salts are salts of an amino group formed with inorganic acids such as hydrochloric acid, hydrobromic acid, phosphoric acid, sulfuric acid and perchloric acid or with organic acids such as acetic acid, maleic acid, tartaric acid, citric acid, succinic acid or malonic acid or by using other methods used in the art such as ion exchange. Other pharmaceutically acceptable salts include, but are not limited to, adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, alkyl having from 1 to 6 carbon atoms, sulfonate and aryl sulfonate.

As used herein, the term “pharmaceutically acceptable ester” refers to esters of a compound formed by the process of the present application which hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include, but are not limited to, formates, acetates, propionates, butyrates, acrylates and ethylsuccinates.

The term “pharmaceutically acceptable prodrugs” as used herein refers to those prodrugs of a compound formed by the process of the present application which are, within the scope of sound medical judgment, suitable for use in contact with the tissues of humans and lower animals with undue toxicity, irritation, allergic response, and the like, commensurate with a reasonable benefit/risk ratio, and effective for their intended use, as well as the zwitterionic forms, where possible, of the compounds of the present application. “Prodrug”, as used herein means a compound which is convertible in vivo by metabolic means (e.g., by hydrolysis) to afford any compound delineated by the formulae of the instant application. Various forms of prodrugs are known in the art, for example, as discussed in Bundgaard, (ed.), Design of Prodrugs, Elsevier (1985); Widder, et al., (ed.), Methods in Enzymology, vol. 4, Academic Press (1985); Krogsgaard-Larsen, et al., (ed). Design and Application of Prodrugs, Textbook of Drug Design and Development, Chapter 5, 113-191 (1991); Bundgaard, et al., Journal of Drug Deliver Reviews, 8:1-38(1992); Bundgaard, J. of Pharmaceutical Sciences, 77:285 et seq. (1988); Higuchi and Stella (eds.) Prodrugs as Novel Drug Delivery Systems, American Chemical Society (1975); and Bernard Testa & Joachim Mayer, “Hydrolysis In Drug And Prodrug Metabolism: Chemistry, Biochemistry And Enzymology,” John Wiley and Sons, Ltd. (2002).

Prodrugs include compounds wherein an amino acid residue, or a polypeptide chain of two or more (e.g., two, three or four) amino acid residues is covalently joined through an amide or ester bond to a free amino, hydroxy or carboxylic acid group of compounds of the application. The amino acid residues include but are not limited to the 20 naturally occurring amino acids commonly designated by three letter symbols and also includes 4-hydroxyproline, hydroxylysine, demosine, isodemosine, 3-methylhistidine, norvalin, beta-alanine, gamma-aminobutyric acid, citrulline, homocysteine, homoserine, ornithine and methionine sulfone. Additional types of prodrugs are also encompassed. For instance, free carboxyl groups can be derivatized as amides or alkyl esters. Free hydroxy groups may be derivatized using groups including but not limited to hemisuccinates, phosphate esters, dimethylaminoacetates, and phosphoryloxymethyloxy carbonyls, as outlined in Advanced Drug Delivery Reviews, 1996, 19, 1 15. Carbamate prodrugs of hydroxy and amino groups are also included, as are carbonate prodrugs, sulfonate esters and sulfate esters of hydroxy groups. Derivatization of hydroxy groups as (acyloxy)methyl and (acyloxy)ethyl ethers wherein the acyl group may be an alkyl ester, optionally substituted with groups including but not limited to ether, amine and carboxylic acid functionalities, or where the acyl group is an amino acid ester as described above, are also encompassed. Prodrugs of this type are described in J. Med Chem. 1996, 39, 10. Free amines can also be derivatized as amides, sulfonamides or phosphonamides. All of these prodrug moieties may incorporate groups including but not limited to ether, amine and carboxylic acid functionalities

Combinations of substituents and variables envisioned by this application are only those that result in the formation of stable compounds. The term “stable”, as used herein, refers to compounds which possess stability sufficient to allow manufacture and which maintains the integrity of the compound for a sufficient period of time to be useful for the purposes detailed herein (e.g., therapeutic or prophylactic administration to a subject).

In addition, some of the compounds of this application have one or more double bonds, or one or more asymmetric centers. Such compounds can occur as racemates, racemic mixtures, single enantiomers, individual diastereomers, diastereomeric mixtures, and cis- or trans- or E- or Z-double isomeric forms, and other stereoisomeric forms that may be defined, in terms of absolute stereochemistry, as (R)- or (S)-, or as (D)- or (L)- for amino acids. All such isomeric forms of these compounds are expressly included in the present application.

“Isomerism” means compounds that have identical molecular formulae but differ in the sequence of bonding of their atoms or in the arrangement of their atoms in space. Isomers that differ in the arrangement of their atoms in space are termed “stereoisomers”. Stereoisomers that are not mirror images of one another are termed “diastereoisomers”, and stereoisomers that are non-superimposable mirror images of each other are termed “enantiomers” or sometimes optical isomers. A mixture containing equal amounts of individual enantiomeric forms of opposite chirality is termed a “racemic mixture”.

A carbon atom bonded to four non-identical substituents is termed a “chiral center”. “Chiral isomer” means a compound with at least one chiral center. Compounds with more than one chiral center may exist either as an individual diastereomer or as a mixture of diastereomers, termed “diastereomeric mixture”. When one chiral center is present, a stereoisomer may be characterized by the absolute configuration (R or S) of that chiral center. Absolute configuration refers to the arrangement in space of the substituents attached to the chiral center. The substituents attached to the chiral center under consideration are ranked in accordance with the Sequence Rule of Cahn, Ingold and Prelog. (Cahn et al., Angew. Chem. Inter. Edit. 1 966, 5, 385; errata 511; Cahn et al., Angew. Chem. 1966, 78, 413; Cahn and Ingold, J. Chem. Soc. 1951 (London), 612; Cahn et a, Experientia 1956, 12, 81; Cahn, J. Chem. Educ. 1964, 41, 116).

“Geometric isomer” means the diastereomers that owe their existence to hindered rotation about double bonds. These configurations are differentiated in their names by the prefixes cis and trans, or Z and E, which indicate that the groups are on the same or opposite side of the double bond in the molecule according to the Cahn-Ingold-Prelog rules.

Furthermore, the structures and other compounds discussed in this application include all atropic isomers thereof. “Atropic isomers” are a type of stereoisomer in which the atoms of two isomers are arranged differently in space. Atropic isomers owe their existence to a restricted rotation caused by hindrance of rotation of large groups about a central bond. Such atropic isomers typically exist as a mixture, however as a result of recent advances in chromatography techniques; it has been possible to separate mixtures of two atropic isomers in select cases.

“Tautomer” is one of two or more structural isomers that exist in equilibrium and is readily converted from one isomeric form to another. This conversion results in the formal migration of a hydrogen atom accompanied by a switch of adjacent conjugated double bonds.

Tautomers exist as a mixture of a tautomeric set in solution. In solid form, usually one tautomer predominates. In solutions where tautomerization is possible, a chemical equilibrium of the tautomers will be reached. The exact ratio of the tautomers depends on several factors, including temperature, solvent and pH. The concept of tautomers that are interconvertable by tautomerizations is called tautomerism.

Of the various types of tautomerism that are possible, two are commonly observed. In keto-enol tautomerism a simultaneous shift of electrons and a hydrogen atom occurs. Ring-chain tautomerism arises as a result of the aldehyde group (—CHO) in a sugar chain molecule reacting with one of the hydroxy groups (—OH) in the same molecule to give it a cyclic (ring-shaped) form as exhibited by glucose. Common tautomeric pairs are: ketone-enol, amide-nitrile, lactam-lactim, amide-imidic acid tautomerism in heterocyclic rings (e.g., in nucleobases such as guanine, thymine and cytosine), amine-enamine and enamine-enamine.

The compounds of this application may also be represented in multiple tautomeric forms, in such instances, the application expressly includes all tautomeric forms of the compounds described herein (e.g., alkylation of a ring system may result in alkylation at multiple sites, the application expressly includes all such reaction products). When the compounds described herein contain olefinic double bonds or other centers of geometric asymmetry, and unless specified otherwise, it is intended that the compounds include both E and Z geometric isomers. Likewise, all tautomeric forms are also intended to be included. The configuration of any carbon-carbon double bond appearing herein is selected for convenience only and is not intended to designate a particular configuration unless the text so states; thus a carbon-carbon double bond depicted arbitrarily herein as trans may be cis, trans, or a mixture of the two in any proportion. All such isomeric forms of such compounds are expressly included in the present application.

In the present specification, the structural formula of the compound represents a certain isomer for convenience in some cases, but the present application includes all isomers, such as geometrical isomers, optical isomers based on an asymmetrical carbon, stereoisomers, tautomers, and the like.

Furthermore, so-called metabolite which is produced by degradation of the present compound in vivo is included in the scope of the present application.

The term “crystal polymorphs”, “polymorphs” or “crystal forms” means crystal structures in which a compound (or a salt or solvate thereof) can crystallize in different crystal packing arrangements, all of which have the same elemental composition. Different crystal forms usually have different X-ray diffraction patterns, infrared spectral, melting points, density hardness, crystal shape, optical and electrical properties, stability and solubility. Recrystallization solvent, rate of crystallization, storage temperature, and other factors may cause one crystal form to dominate. Crystal polymorphs of the compounds can be prepared by crystallization under different conditions.

Additionally, the compounds of the present application, for example, the salts of the compounds, can exist in either hydrated or unhydrated (the anhydrous) form or as solvates with other solvent molecules. Non-limiting examples of hydrates include monohydrates, dihydrates, etc. Non-limiting examples of solvates include ethanol solvates, acetone solvates, etc.

“Solvate” means solvent addition forms that contain either stoichiometric or non stoichiometric amounts of solvent. Some compounds have a tendency to trap a fixed molar ratio of solvent molecules in the crystalline solid state, thus forming a solvate. If the solvent is water the solvate formed is a hydrate; and if the solvent is alcohol, the solvate formed is an alcoholate. Hydrates are formed by the combination of one or more molecules of water with one molecule of the substance in which the water retains its molecular state as H₂O.

### Method of Synthesizing the Compounds

The compounds of the present application (e.g., a compound of Formula Ia, Ib, or I′) may be made by a variety of methods, including standard chemistry. The synthetic processes of the application can tolerate a wide variety of functional groups, therefore various substituted starting materials can be used. The processes generally provide the desired final compound at or near the end of the overall process, although it may be desirable in certain instances to further convert the compound to a pharmaceutically acceptable salt, ester or prodrug thereof. Suitable synthetic routes are depicted in the schemes below.

Compounds of the present application can be prepared in a variety of ways using commercially available starting materials, compounds known in the literature, or from readily prepared intermediates, by employing standard synthetic methods and procedures either known to those skilled in the art, or which will be apparent to the skilled artisan in light of the teachings herein. Standard synthetic methods and procedures for the preparation of organic molecules and functional group transformations and manipulations can be obtained from the relevant scientific literature or from standard textbooks in the field. Although not limited to any one or several sources, classic texts such as Smith, M. B., March, J., March's Advanced Organic Chemistry: Reactions, Mechanisms, and Structure, 5^{th} edition, John Wiley & Sons: New York, 2001; and Greene, T. W., Wuts, P. G. M., Protective Groups in Organic Synthesis, 3^{rd} edition, John Wiley & Sons: New York, 1999, incorporated by reference herein, are useful and recognized reference textbooks of organic synthesis known to those in the art. The following descriptions of synthetic methods are designed to illustrate, but not to limit, general procedures for the preparation of compounds of the present application.

The compounds of disclosed herein may be prepared by methods known in the art of organic synthesis as set forth in part by the following synthetic schemes. In the schemes described below, it is well understood that protecting groups for sensitive or reactive groups are employed where necessary in accordance with general principles or chemistry. Protecting groups are manipulated according to standard methods of organic synthesis (T. W. Greene and P. G. M. Wuts, “Protective Groups in Organic Synthesis”, Third edition, Wiley, New York 1999). These groups are removed at a convenient stage of the compound synthesis using methods that are readily apparent to those skilled in the art. The selection processes, as well as the reaction conditions and order of their execution, shall be consistent with the preparation of compounds of disclosed herein.

Those skilled in the art will recognize if a stereocenter exists in the compounds of disclosed herein. Accordingly, the present application includes both possible stereoisomers (unless specified in the synthesis) and includes not only racemic compounds but the individual enantiomers and/or diastereomers as well. When a compound is desired as a single enantiomer or diastereomer, it may be obtained by stereospecific synthesis or by resolution of the final product or any convenient intermediate. Resolution of the final product, an intermediate, or a starting material may be affected by any suitable method known in the art. See, for example, “Stereochemistry of Organic Compounds” by E. L. Eliel, S. H. Wilen, and L. N. Mander (Wiley-Interscience, 1994).

All the abbreviations used in this application are found in “Protective Groups in Organic Synthesis” by John Wiley & Sons, Inc, or the MERCK INDEX by MERCK & Co., Inc, or other chemistry books or chemicals catalogs by chemicals vendor such as Aldrich, or according to usage know in the art.

The compounds of the present application can be prepared in a number of ways well known to those skilled in the art of organic synthesis, such as those described in U.S. Pat. No. 8,946,235 and WO 2017/004383. By way of example, compounds of the present application can be synthesized using the methods described below, together with synthetic methods known in the art of synthetic organic chemistry, or variations thereon as appreciated by those skilled in the art. Preferred methods include but are not limited to those methods described below.

A mixture of enantiomers, diastereomers, and/or cis/trans isomers resulting from the processes described above can be separated into their single components by chiral salt technique, chromatography using normal phase, or reverse phase or chiral column, depending on the nature of the separation.

It should be understood that in the description and formulae shown above, the various groups and other variables are as defined herein, except where otherwise indicated. Furthermore, for synthetic purposes, the compounds of General Schemes are mere representatives with elected radicals to illustrate the general synthetic methodology of the compounds of disclosed herein.

A compound of the application can be prepared as a pharmaceutically acceptable acid addition salt by reacting the free base form of the compound with a pharmaceutically acceptable inorganic or organic acid. Alternatively, a pharmaceutically acceptable base addition salt of a compound of the application can be prepared by reacting the free acid form of the compound with a pharmaceutically acceptable inorganic or organic base. Alternatively, the salt forms of the compounds of the application can be prepared using salts of the starting materials or intermediates.

The free acid or free base forms of the compounds of the application can be prepared from the corresponding base addition salt or acid addition salt from, respectively. For example a compound of the application in an acid addition salt form can be converted to the corresponding free base by treating with a suitable base (e.g., ammonium hydroxide solution, sodium hydroxide, and the like). A compound of the application in a base addition salt form can be converted to the corresponding free acid by treating with a suitable acid (e.g., hydrochloric acid, etc.).

Prodrugs of the compounds of the application can be prepared by methods known to those of ordinary skill in the art (e.g., for further details see Saulnier et al., (1994), Bioorganic and Medicinal Chemistry Letters, Vol. 4, p. 1985). For example, appropriate prodrugs can be prepared by reacting a non-derivatized compound of the application with a suitable carbamylating agent (e.g., 1,1-acyloxyalkylcarbanochloridate, para-nitrophenyl carbonate, or the like).

Protected derivatives of the compounds of the application can be made by means known to those of ordinary skill in the art. A detailed description of techniques applicable to the creation of protecting groups and their removal can be found in T. W. Greene, “Protecting Groups in Organic Chemistry”, 3rd edition, John Wiley and Sons, Inc., 1999.

Compounds of the present application can be conveniently prepared, or formed during the process of the application, as solvates (e.g., hydrates). Hydrates of compounds of the present application can be conveniently prepared by recrystallization from an aqueous/organic solvent mixture, using organic solvents such as dioxin, tetrahydrofuran or methanol.

Acids and bases useful in the methods herein are known in the art. Acid catalysts are any acidic chemical, which can be inorganic (e.g., hydrochloric, sulfuric, nitric acids, aluminum trichloride) or organic (e.g., camphorsulfonic acid, p-toluenesulfonic acid, acetic acid, ytterbium triflate) in nature. Acids are useful in either catalytic or stoichiometric amounts to facilitate chemical reactions. Bases are any basic chemical, which can be inorganic (e.g., sodium bicarbonate, potassium hydroxide) or organic (e.g., triethylamine, pyridine) in nature. Bases are useful in either catalytic or stoichiometric amounts to facilitate chemical reactions.

Optical isomers may be prepared from their respective optically active precursors by the procedures described herein, or by resolving the racemic mixtures. The resolution can be carried out in the presence of a resolving agent, by chromatography or by repeated crystallization or by some combination of these techniques which are known to those skilled in the art. Further details regarding resolutions can be found in Jacques, et al., Enantiomers, Racemates, and Resolutions (John Wiley & Sons, 1981).

The synthesized compounds can be separated from a reaction mixture and further purified by a method such as column chromatography, high pressure liquid chromatography, or recrystallization. As can be appreciated by the skilled artisan, further methods of synthesizing the compounds of the formulae herein will be evident to those of ordinary skill in the art. Additionally, the various synthetic steps may be performed in an alternate sequence or order to give the desired compounds. In addition, the solvents, temperatures, reaction durations, etc. delineated herein are for purposes of illustration only and one of ordinary skill in the art will recognize that variation of the reaction conditions can produce the desired bridged macrocyclic products of the present application. Synthetic chemistry transformations and protecting group methodologies (protection and deprotection) useful in synthesizing the compounds described herein are known in the art and include, for example, those such as described in R. Larock, Comprehensive Organic Transformations, VCH Publishers (1989); T. W. Greene and P. G. M. Wuts, Protective Groups in Organic Synthesis, 2d. Ed., John Wiley and Sons (1991); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995), and subsequent editions thereof.

The compounds of this application may be modified by appending various functionalities via any synthetic means delineated herein to enhance selective biological properties. Such modifications are known in the art and include those which increase biological penetration into a given biological system (e.g., blood, lymphatic system, central nervous system), increase oral availability, increase solubility to allow administration by injection, alter metabolism and alter rate of excretion.

### Biological Assays

### Biochemical Assays

EGFR biochemical assays are carried out using a homogeneous time-resolved fluorescence (HTRF) assay. The reaction mixtures contain biotin-Lck-peptide substrate, wild type, or mutant EGFR enzyme in reaction buffer. Enzyme concentrations are adjusted to accommodate varying kinase activity and ATP concentrations. Pharmaceutical combinations or compounds of the present application are diluted into the assay mixture and IC₅₀ values are determined using 12-point inhibition curves.

### Phospho-EGFR Target Modulation Assays and ELISA

Cells are lysed with lysis buffer containing protease and phosphatase inhibitors and the plates are shaken. An aliquot from each well is then transferred to prepared ELISA plates for analysis. Once harvested and plated, the cells are pre-treated with media with or without EGF. The pharmaceutical combinations or compounds of the present application are then added and IC₅₀ values are determined using an EGFR biochemical assay described above.

Solid high-binding ELISA plates are coated with goat anti-EGFR capture antibody. Plates are then blocked with BSA in a buffer, and then washed. Aliquots of lysed cell are added to each well of the ELISA plate and the plate is incubated. An anti-phospho-EGFR is then added and is followed by further incubation. After washing, anti-rabbit-HRP is added and the plate is again incubated. Chemiluminescent detection is carried out with SuperSignal ELISA Pico substrate. Signal is read on EnVision plate reader using built-in UltraLUM setting.

### Western Blotting

Cell lysates are equalized to protein content and loaded onto a gel with running buffer. Membranes are probed with primary antibodies and are then washed. HRP-conjugated secondary antibodies are added and after washing. HRP is detected using a HRP substrate reagent and recorded with an imager.

### Cell Proliferation Assays

Cell lines are plated in media. The pharmaceutical combinations or compounds of the present application are then serially diluted and transferred to the cells. Cell viability is measured via a luminescent readout. Data is analyzed by non-linear regression curve-fitting.

### Methods of the Application

In another aspect, the application provides a method of inhibiting a kinase, comprising contacting the kinase with an effective amount of a pharmaceutical combination, as described herein, or an effective amount of an allosteric EGFR inhibitor, as described herein, in combination with (e.g., in temporal proximity with) an effective amount of an ATP-competitive EGFR inhibitor, as described herein. In some embodiments, the kinase comprises a mutated cysteine residue. In further embodiments, the mutated cysteine residue is located in or near the position equivalent to Cys 797 in EGFR, including such position in Jak3, Blk, Bmx, Btk, HER2 (ErbB2), HER4 (ErbB4), Itk, Tec, and Txk. In some embodiments, the kinase is EGFR. In some embodiments, the kinase is a Her-kinase.

In another aspect, the application provides a method of inhibiting EGFR, comprising contacting the kinase with an effective amount of a pharmaceutical combination, as described herein, or an effective amount of an allosteric EGFR inhibitor, as described herein, in combination with (e.g., in temporal proximity with) an effective amount of an ATP-competitive EGFR inhibitor, as described herein. In some embodiments, the EGFR comprises one or more mutations, as described herein.

Another aspect of the application provides a method of treating or preventing a disease, comprising administering to a subject in need thereof an effective amount of a pharmaceutical combination, as described herein, or an effective amount of an allosteric EGFR inhibitor, as described herein, in combination with (e.g., in temporal proximity with) an effective amount of an ATP-competitive EGFR inhibitor, as described herein. In some embodiments, the disease is mediated by a kinase. In further embodiments, the kinase comprises a mutated cysteine residue. In further embodiments, the mutated cysteine residue is located in or near the position equivalent to Cys 797 in EGFR, including such positions in Jak3, Blk, Bmx, Btk, HER2 (ErbB2), HER4 (ErbB4), Itk, Tec, and Txk. In some embodiments, the disease is mediated by EGFR (e.g., EGFR plays a role in the initiation or development of the disease). In further embodiments, the EGFR is a Her-kinase. In further embodiments, the Her-kinase is HER1, HER2, or HER4.

Another aspect of the application provides a method of treating or preventing a disease, comprising administering to a subject in need thereof an effective amount of a pharmaceutical combination, as described herein, or an effective amount of an allosteric EGFR inhibitor, as described herein, in combination with (e.g., in temporal proximity with) an effective amount of an ATP-competitive EGFR inhibitor, as described herein. In some embodiments, the disease is mediated by EGFR. In some embodiments, the EGFR comprises one or more mutations, as described herein.

In certain embodiments, the disease is cancer or a proliferation disease. In further embodiments, the disease is lung cancer, colon cancer, breast cancer, prostate cancer, liver cancer, pancreas cancer, brain cancer, kidney cancer, ovarian cancer, stomach cancer, skin cancer, bone cancer, gastric cancer, breast cancer, pancreatic cancer, glioma, glioblastoma, hepatocellular carcinoma, papillary renal carcinoma, head and neck squamous cell carcinoma, leukemias, lymphomas, myelomas, or solid tumors.

In other embodiments, the disease is inflammation, arthritis, rheumatoid arthritis, spondyiarthropathies, gouty arthritis, osteoarthritis, juvenile arthritis, and other arthritic conditions, systemic lupus erthematosus (SLE), skin-related conditions, psoriasis, eczema, burns, dermatitis, neuroinflammation, allergy, pain, neuropathic pain, fever, pulmonary disorders, lung inflammation, adult respiratory distress syndrome, pulmonary sarcoisosis, asthma, silicosis, chronic pulmonary inflammatory disease, and chronic obstructive pulmonary disease (COPD), cardiovascular disease, arteriosclerosis, myocardial infarction (including post-myocardial infarction indications), thrombosis, congestive heart failure, cardiac reperfusion injury, as well as complications associated with hypertension and/or heart failure such as vascular organ damage, restenosis, cardiomyopathy, stroke including ischemic and hemorrhagic stroke, reperfusion injury, renal reperfusion injury, ischemia including stroke and brain ischemia, and ischemia resulting from cardiac/coronary bypass, neurodegenerative disorders, liver disease and nephritis, gastrointestinal conditions, inflammatory bowel disease, Crohn's disease, gastritis, irritable bowel syndrome, ulcerative colitis, ulcerative diseases, gastric ulcers, viral and bacterial infections, sepsis, septic shock, gram negative sepsis, malaria, meningitis, HIV infection, opportunistic infections, cachexia secondary to infection or malignancy, cachexia secondary to acquired immune deficiency syndrome (AIDS), AIDS, ARC (AIDS related complex), pneumonia, herpes virus, myalgias due to infection, influenza, autoimmune disease, graft vs. host reaction and allograft rejections, treatment of bone resorption diseases, osteoporosis, multiple sclerosis, cancer, leukemia, lymphoma, colorectal cancer, brain cancer, bone cancer, epithelial call-derived neoplasia (epithelial carcinoma), basal cell carcinoma, adenocarcinoma, gastrointestinal cancer, lip cancer, mouth cancer, esophageal cancer, small bowel cancer, stomach cancer, colon cancer, liver cancer, bladder cancer, pancreas cancer, ovarian cancer, cervical cancer, lung cancer, breast cancer, skin cancer, squamus cell and/or basal cell cancers, prostate cancer, renal cell carcinoma, and other known cancers that affect epithelial cells throughout the body, chronic myelogenous leukemia (CML), acute myeloid leukemia (AML) and acute promyelocytic leukemia (APL), angiogenesis including neoplasia, metastasis, central nervous system disorders, central nervous system disorders having an inflammatory or apoptotic component, Alzheimer's disease, Parkinson's disease, Huntington's disease, amyotrophic lateral sclerosis, spinal cord injury, and peripheral neuropathy, or B-Cell Lymphoma.

In further embodiments, the disease is inflammation, arthritis, rheumatoid arthritis, spondylarthropathies, gouty arthritis, osteoarthritis, juvenile arthritis, and other arthritic conditions, systemic lupus erthematosus (SLE), skin-related conditions, psoriasis, eczema, dermatitis, pain, pulmonary disorders, lung inflammation, adult respiratory distress syndrome, pulmonary sarcoisosis, asthma, chronic pulmonary inflammatory disease, and chronic obstructive pulmonary disease (COPD), cardiovascular disease, arteriosclerosis, myocardial infarction (including post-myocardial infarction indications), congestive heart failure, cardiac reperfusion injury, inflammatory bowel disease, Crohn's disease, gastritis, irritable bowel syndrome, leukemia or lymphoma.

In another aspect, the application provides a method of treating or preventing cancer, wherein the cancer cell comprise activated EGFR, comprising administering to a subject in need thereof an effective amount of a pharmaceutical combination, as described herein, or an effective amount of an allosteric EGFR inhibitor, as described herein, in combination with (e.g., in temporal proximity with) an effective amount of an ATP-competitive EGFR inhibitor, as described herein.

In certain embodiments, the EGFR activation is selected from mutation of EGFR, amplification of EGFR, expression of EGFR, and ligand mediated activation of EGFR.

Another aspect of the application provides a method of treating or preventing cancer in a subject, wherein the subject is identified as being in need of EGFR inhibition for the treatment of cancer, comprising administering to the subject an effective amount of a pharmaceutical combination, as described herein, or an effective amount of an allosteric EGFR inhibitor, as described herein, in combination with (e.g., in temporal proximity with) an effective amount of an ATP-competitive EGFR inhibitor, as described herein.

In certain embodiments, the subject identified as being in need of EGFR inhibition is resistant to a known EGFR inhibitor, including but not limited to, gefitinib, erlotinib, afatinib, AZD9291, CO-1686, or WZ4002. In certain embodiments, a diagnostic test is performed to determine if the subject has an activating mutation in EGFR. In certain embodiments, a diagnostic test is performed to determine if the subject has an EGFR harboring an activating and a drug resistance mutation, such as those described herein. Activating mutations comprise without limitation L858R, G719S, G719C, G719A, L718Q, L861Q, a deletion in exon 19 and/or an insertion in exon 20. Drug resistant EGFR mutants can have without limitation a drug resistance mutation comprising T790M, T854A, L718Q, C797S, or D761Y. The diagnostic test can comprise sequencing, pyrosequencing, PCR RT-PCR, or similar analysis techniques known to those of skill in the art that can detect nucleotide sequences.

In another aspect, the application provides a method of treating or preventing cancer, wherein the cancer cell comprises an activated ERBB2, comprising administering to a subject in need thereof an effective amount of a pharmaceutical combination, as described herein, or an effective amount of an allosteric EGFR inhibitor, as described herein, in combination with (e.g., in temporal proximity with) an effective amount of an ATP-competitive EGFR inhibitor, as described herein. In certain embodiments, the ERBB2 activation is selected from mutation of ERBB2, expression of ERBB2 and amplification of ERBB2. In further embodiments, the mutation is a mutation in exon 20 of ERBB2.

In another aspect, the application provides a method of treating cancer in a subject, wherein the subject is identified as being in need of ERBB2 inhibition for the treatment of cancer, comprising administering to the subject in need thereof an effective amount of a pharmaceutical combination, as described herein, or an effective amount of an allosteric EGFR inhibitor, as described herein, in combination with (e.g., in temporal proximity with) an effective amount of an ATP-competitive EGFR inhibitor, as described herein.

Another aspect of the application provides a method of preventing resistance to a known EGFR inhibitor, including but not limited to, gefitinib, erotinib, afatinib, lapatinib, neratinib, WZ4002, CL-387785, AZD9291, and CO-1686, in a disease, comprising administering to a subject in need thereof an effective amount of a pharmaceutical combination, as described herein, or an effective amount of an allosteric EGFR inhibitor, as described herein, in combination with (e.g., in temporal proximity with) an effective amount of an ATP-competitive EGFR inhibitor, as described herein.

In certain embodiments, the application provides a method of treating any of the disorders described herein, wherein the subject is a human. In certain embodiments, the application provides a method of preventing any of the disorders described herein, wherein the subject is a human.

In some embodiments, the methods of application further comprises administering a second agent. In some embodiments, the second agent prevents EGFR dimer formation. In some embodiments, the second agent that prevents EGFR dimer formation is an antibody. In further embodiments, the second agent that prevents EGFR dimer formation is cetuximab, trastuzumab, or panitumumab. In further embodiments, the second agent that prevents EGFR dimer formation is cetuximab.

In some embodiments, the allosteric EGFR inhibitor, as described herein, and the ATP-competitive EGFR inhibitor, as described herein, are administered simultaneously or sequentially. In further embodiments, the allosteric EGFR inhibitor, as described herein, are administered prior to or subsequent to the ATP-competitive EGFR inhibitor.

In some embodiments, the allosteric EGFR inhibitor, as described herein, and the ATP-competitive EGFR inhibitor, as described herein, are administered in temporal proximity. In some embodiments, the allosteric EGFR inhibitor, as described herein, is used in combination (e.g., in a combinational therapy) with the ATP-competitive EGFR inhibitor, as described herein, wherein the administration of the allosteric EGFR inhibitor and the administration of the ATP-competitive EGFR inhibitor occurs in temporal proximity.

In some embodiments, “temporal proximity” means that administration of one therapeutic agent occurs within a time period before or after the administration of another therapeutic agent, such that the therapeutic effect of the one therapeutic agent overlaps with the therapeutic effect of the another therapeutic agent. In some embodiments, the therapeutic effect of the one therapeutic agent completely overlaps with the therapeutic effect of the another therapeutic agent. In some embodiments, “temporal proximity” means that administration of one therapeutic agent occurs within a time period before or after the administration of another therapeutic agent, such that there is a synergistic effect between the one therapeutic agent and the another therapeutic agent. “Temporal proximity” may vary according to various factors, including but not limited to, the age, gender, weight, genetic background, medical condition, disease history, and treatment history of the subject to which the therapeutic agents are to be administered; the disease or condition to be treated or ameliorated; the therapeutic outcome to be achieved; the dosage, dosing frequency, and dosing duration of the therapeutic agents; the pharmacokinetics and pharmacodynamics of the therapeutic agents; and the route(s) through which the therapeutic agents are administered. In some embodiments, “temporal proximity” means within 15 minutes, within 30 minutes, within an hour, within two hours, within four hours, within six hours, within eight hours, within 12 hours, within 18 hours, within 24 hours, within 36 hours, within 2 days, within 3 days, within 4 days, within 5 days, within 6 days, within a week, within 2 weeks, within 3 weeks, within 4 weeks, with 6 weeks, or within 8 weeks. In some embodiments, multiple administration of one therapeutic agent can occur in temporal proximity to a single administration of another therapeutic agent. In some embodiments, temporal proximity may change during a treatment cycle or within a dosing regimen.

In other embodiments, the allosteric EGFR inhibitor, as described herein, and the ATP-competitive EGFR inhibitor, as described herein, and the additional therapeutic agent are administered simultaneously or sequentially.

In another aspect, the application provides an allosteric EGFR inhibitor, as described herein, for use in combination (e.g., in a combinational therapy) with an ATP-competitive EGFR inhibitor, as described herein, and optionally further in combination with a second agent that prevents EGFR dimer formation, for

inhibiting a kinase (e.g., EGFR) in a subject in need thereof,

treating or preventing a disease (e.g., a disease in which EGFR plays a role) in a subject in need thereof,

treating or preventing a disease resistant to an EGFR targeted therapy, such as a therapy with gefitinib, erlotinib, afatinib, AZD9291, CO-1686, or WZ4002, in a subject in need thereof,

treating or preventing cancer in a subject in need thereof, wherein the cell of the cancer comprises an activated EGFR or an activated ERBB2, or

treating or preventing cancer in a subject, wherein the subject is identified as being in need of EGFR inhibition or ERBB2 inhibition for the treatment or prevention of cancer.

In some embodiments, the second agent that prevents EGFR dimer formation is an antibody. In further embodiments, the second agent that prevents EGFR dimer formation is cetuximab, trastuzumab, or panitumumab. In further embodiments, the second agent that prevents EGFR dimer formation is cetuximab.

In another aspect, the application provides use of an allosteric EGFR inhibitor, as described herein, in combination (e.g., in a combinational therapy) with an ATP-competitive EGFR inhibitor, as described herein, and optionally further in combination with a second agent that prevents EGFR dimer formation, for

inhibiting a kinase (e.g., EGFR) in a subject in need thereof,

treating or preventing a disease (e.g., a disease in which EGFR plays a role) in a subject in need thereof,

treating or preventing a disease resistant to an EGFR targeted therapy, such as a therapy with gefitinib, erlotinib, afatinib, AZD9291, CO-1686, or WZ4002, in a subject in need thereof,

treating or preventing cancer in a subject in need thereof, wherein the cell of the cancer comprises an activated EGFR or an activated ERBB2, or

treating or preventing cancer in a subject, wherein the subject is identified as being in need of EGFR inhibition or ERBB2 inhibition for the treatment or prevention of cancer.

In some embodiments, the second agent that prevents EGFR dimer formation is an antibody. In further embodiments, the second agent that prevents EGFR dimer formation is cetuximab, trastuzumab, or panitumumab. In further embodiments, the second agent that prevents EGFR dimer formation is cetuximab.

In another aspect, the application provides a combination (e.g., a therapeutic combination) of an allosteric EGFR inhibitor, as described herein, and an ATP-competitive EGFR inhibitor, as described herein, and optionally further in combination with a second agent that prevents EGFR dimer formation, for

inhibiting a kinase (e.g., EGFR) in a subject in need thereof,

treating or preventing a disease (e.g., a disease in which EGFR plays a role) in a subject in need thereof,

treating or preventing a disease resistant to an EGFR targeted therapy, such as a therapy with gefitinib, erlotinib, afatinib, AZD9291, CO-1686, or WZ4002, in a subject in need thereof,

treating or preventing cancer in a subject in need thereof, wherein the cell of the cancer comprises an activated EGFR or an activated ERBB2, or

treating or preventing cancer in a subject, wherein the subject is identified as being in need of EGFR inhibition or ERBB2 inhibition for the treatment or prevention of cancer.

In some embodiments, the second agent that prevents EGFR dimer formation is an antibody. In further embodiments, the second agent that prevents EGFR dimer formation is cetuximab, trastuzumab, or panitumumab. In further embodiments, the second agent that prevents EGFR dimer formation is cetuximab.

In another aspect, the application provides use of a combination (e.g., a therapeutic combination) of an allosteric EGFR inhibitor, as described herein, and an ATP-competitive EGFR inhibitor, as described herein, and optionally further a second agent that prevents EGFR dimer formation, for

inhibiting a kinase (e.g., EGFR) in a subject in need thereof,

treating or preventing a disease (e.g., a disease in which EGFR plays a role) in a subject in need thereof,

treating or preventing a disease resistant to an EGFR targeted therapy, such as a therapy with gefitinib, erlotinib, afatinib, AZD9291, CO-1686, or WZ4002, in a subject in need thereof,

treating or preventing cancer in a subject in need thereof, wherein the cell of the cancer comprises an activated EGFR or an activated ERBB2, or

treating or preventing cancer in a subject, wherein the subject is identified as being in need of EGFR inhibition or ERBB2 inhibition for the treatment or prevention of cancer.

In some embodiments, the second agent that prevents EGFR dimer formation is an antibody. In further embodiments, the second agent that prevents EGFR dimer formation is cetuximab, trastuzumab, or panitumumab. In further embodiments, the second agent that prevents EGFR dimer formation is cetuximab.

In another aspect, the application provides a combination (e.g., a therapeutic combination) of an allosteric EGFR inhibitor, as described herein, and an ATP-competitive EGFR inhibitor, as described herein, and optionally further a second agent that prevents EGFR dimer formation, for use in the manufacture of a medicament for

inhibiting a kinase (e.g., EGFR) in a subject in need thereof,

treating or preventing a disease (e.g., a disease in which EGFR plays a role) in a subject in need thereof,

treating or preventing a disease resistant to an EGFR targeted therapy, such as a therapy with gefitinib, erlotinib, afatinib, AZD9291, CO-1686, or WZ4002, in a subject in need thereof,

treating or preventing cancer in a subject in need thereof, wherein the cell of the cancer comprises an activated EGFR or an activated ERBB2, or

treating or preventing cancer in a subject, wherein the subject is identified as being in need of EGFR inhibition or ERBB2 inhibition for the treatment or prevention of cancer.

In some embodiments, the second agent that prevents EGFR dimer formation is an antibody. In further embodiments, the second agent that prevents EGFR dimer formation is cetuximab, trastuzumab, or panitumumab. In further embodiments, the second agent that prevents EGFR dimer formation is cetuximab.

In another aspect, the application provides use of a combination (e.g., a therapeutic combination) of an allosteric EGFR inhibitor, as described herein, and an ATP-competitive EGFR inhibitor, as described herein, and optionally further a second agent that prevents EGFR dimer formation, for use in the manufacture of a medicament for

inhibiting a kinase (e.g., EGFR) in a subject in need thereof,

treating or preventing a disease (e.g., a disease in which EGFR plays a role) in a subject in need thereof,

treating or preventing a disease resistant to an EGFR targeted therapy, such as a therapy with gefitinib, erlotinib, afatinib, AZD9291, CO-1686, or WZ4002, in a subject in need thereof,

treating or preventing cancer in a subject in need thereof, wherein the cell of the cancer comprises an activated EGFR or an activated ERBB2, or

treating or preventing cancer in a subject, wherein the subject is identified as being in need of EGFR inhibition or ERBB2 inhibition for the treatment or prevention of cancer.

Another aspect of the present application relates to a pharmaceutical combination, as described herein, optionally in combination with a second agent that prevents EGFR dimer formation, for

inhibiting a kinase (e.g., EGFR) in a subject in need thereof,

treating or preventing a disease (e.g., a disease in which EGFR plays a role) in a subject in need thereof,

treating or preventing a disease resistant to an EGFR targeted therapy, such as a therapy with gefitinib, erlotinib, afatinib, AZD9291, CO-1686, or WZ4002, in a subject in need thereof,

treating or preventing cancer in a subject in need thereof, wherein the cell of the cancer comprises an activated EGFR or an activated ERBB2, or

treating or preventing cancer in a subject, wherein the subject is identified as being in need of EGFR inhibition or ERBB2 inhibition for the treatment or prevention of cancer.

Another aspect of the present application relates to use of a pharmaceutical combination, as described herein, optionally in combination with a second agent that prevents EGFR dimer formation, for

inhibiting a kinase (e.g., EGFR) in a subject in need thereof,

treating or preventing a disease (e.g., a disease in which EGFR plays a role) in a subject in need thereof,

treating or preventing a disease resistant to an EGFR targeted therapy, such as a therapy with gefitinib, erlotinib, afatinib, AZD9291, CO-1686, or WZ4002, in a subject in need thereof,

treating or preventing cancer in a subject in need thereof, wherein the cell of the cancer comprises an activated EGFR or an activated ERBB2, or

treating or preventing cancer in a subject, wherein the subject is identified as being in need of EGFR inhibition or ERBB2 inhibition for the treatment or prevention of cancer.

Another aspect of the present application relates to a pharmaceutical combination, as described herein, optionally in combination with a second agent that prevents EGFR dimer formation, for use in the manufacture of a medicament for

inhibiting a kinase (e.g., EGFR) in a subject in need thereof,

treating or preventing a disease (e.g., a disease in which EGFR plays a role) in a subject in need thereof,

treating or preventing a disease resistant to an EGFR targeted therapy, such as a therapy with gefitinib, erlotinib, afatinib, AZD9291, CO-1686, or WZ4002, in a subject in need thereof,

treating or preventing cancer in a subject in need thereof, wherein the cell of the cancer comprises an activated EGFR or an activated ERBB2, or

treating or preventing cancer in a subject, wherein the subject is identified as being in need of EGFR inhibition or ERBB2 inhibition for the treatment or prevention of cancer.

Another aspect of the present application relates to use of a pharmaceutical combination, as described herein, optionally in combination with a second agent that prevents EGFR dimer formation, in the manufacture of a medicament for

inhibiting a kinase (e.g., EGFR) in a subject in need thereof,

treating or preventing a disease (e.g., a disease in which EGFR plays a role) in a subject in need thereof,

treating or preventing a disease resistant to an EGFR targeted therapy, such as a therapy with gefitinib, erlotinib, afatinib, AZD9291, CO-1686, or WZ4002, in a subject in need thereof,

treating or preventing cancer in a subject in need thereof, wherein the cell of the cancer comprises an activated EGFR or an activated ERBB2, or

treating or preventing cancer in a subject, wherein the subject is identified as being in need of EGFR inhibition or ERBB2 inhibition for the treatment or prevention of cancer.

In some embodiments, in the methods, pharmaceutical combinations or compositions, pharmaceutical combinations or compositions for use, or uses of the pharmaceutical combinations or compositions, as described herein, the allosteric EGFR inhibitor is Compound A or a pharmaceutically acceptable salt, hydrate, and solvate thereof, and the ATP-competitive inhibitor is Compound O or a pharmaceutically acceptable salt, hydrate, and solvate thereof.

In some embodiments, the second agent that prevents EGFR dimer formation is an antibody. In further embodiments, the second agent that prevents EGFR dimer formation is cetuximab, trastuzumab, or panitumumab. In further embodiments, the second agent that prevents EGFR dimer formation is cetuximab.

As inhibitors of EGFR kinases, the compounds, combinations, and compositions of this application are particularly useful for treating or lessening the severity of a disease, condition, or disorder where a protein kinase is implicated in the disease, condition, or disorder. In one aspect, the present application provides a method for treating or lessening the severity of a disease, condition, or disorder where a protein kinase is implicated in the disease state. In another aspect, the present application provides a method for treating or lessening the severity of a kinase disease, condition, or disorder where inhibition of enzymatic activity is implicated in the treatment of the disease. In another aspect, this application provides a method for treating or lessening the severity of a disease, condition, or disorder with compounds, combinations, and compositions that inhibit enzymatic activity by binding to the protein kinase. Another aspect provides a method for treating or lessening the severity of a kinase disease, condition, or disorder by inhibiting enzymatic activity of the kinase with a protein kinase inhibitor.

In some embodiments, the method is used to treat or prevent a condition selected from autoimmune diseases, inflammatory diseases, proliferative and hyperproliferative diseases, immunologically-mediated diseases, bone diseases, metabolic diseases, neurological and neurodegenerative diseases, cardiovascular diseases, hormone related diseases, allergies, asthma, and Alzheimer's disease. In other embodiments, the condition is selected from a proliferative disorder and a neurodegenerative disorder.

One aspect of this application provides compounds, combinations, and compositions that are useful for the treatment of diseases, disorders, and conditions characterized by excessive or abnormal cell proliferation. Such diseases include, but are not limited to, a proliferative or hyperproliferative disease, and a neurodegenerative disease. Examples of proliferative and hyperproliferative diseases include, without limitation, cancer. The term “cancer” includes, but is not limited to, the following cancers: breast; ovary; cervix; prostate; testis, genitourinary tract; esophagus; larynx, glioblastoma; neuroblastoma; stomach; skin, keratoacanthoma; lung, epidermoid carcinoma, large cell carcinoma, small cell carcinoma, lung adenocarcinoma; bone; colon; colorectal; adenoma; pancreas, adenocarcinoma; thyroid, follicular carcinoma, undifferentiated carcinoma, papillary carcinoma; seminoma; melanoma; sarcoma; bladder carcinoma; liver carcinoma and biliary passages; kidney carcinoma; myeloid disorders; lymphoid disorders, Hodgkin's, hairy cells; buccal cavity and pharynx (oral), lip, tongue, mouth, pharynx; small intestine; colonrectum, large intestine, rectum, brain and central nervous system; chronic myeloid leukemia (CML), and leukemia. The term “cancer” includes, but is not limited to, the following cancers: myeloma, lymphoma, or a cancer selected from gastric, renal, or and the following cancers: head and neck, oropharangeal, non-small cell lung cancer (NSCLC), endometrial, hepatocarcinoma, Non-Hodgkins lymphoma, and pulmonary.

The term “cancer” refers to any cancer caused by the proliferation of malignant neoplastic cells, such as tumors, neoplasms, carcinomas, sarcomas, leukemias, lymphomas and the like. For example, cancers include, but are not limited to, mesothelioma, leukemias and lymphomas such as cutaneous T-cell lymphomas (CTCL), noncutaneous peripheral T-cell lymphomas, lymphomas associated with human T-cell lymphotrophic virus (HTLV) such as adult T-cell leukemia/lymphoma (ATLL), B-cell lymphoma, acute nonlymphocytic leukemias, chronic lymphocytic leukemia, chronic myelogenous leukemia, acute myelogenous leukemia, lymphomas, and multiple myeloma, non-Hodgkin lymphoma, acute lymphatic leukemia (ALL), chronic lymphatic leukemia (CLL), Hodgkin's lymphoma, Burkitt lymphoma, adult T-cell leukemia lymphoma, acute-myeloid leukemia (AML), chronic myeloid leukemia (CML), or hepatocellular carcinoma. Further examples include myelodisplastic syndrome, childhood solid tumors such as brain tumors, neuroblastoma, retinoblastoma, Wilms' tumor, bone tumors, and soft-tissue sarcomas, common solid tumors of adults such as head and neck cancers (e.g., oral, laryngeal, nasopharyngeal and esophageal), genitourinary cancers (e.g., prostate, bladder, renal, uterine, ovarian, testicular), lung cancer (e.g., small-cell and non-small cell), breast cancer, pancreatic cancer, melanoma and other skin cancers, stomach cancer, brain tumors, tumors related to Gorlin's syndrome (e.g., medulloblastoma, meningioma, etc.), and liver cancer. Additional exemplary forms of cancer which may be treated by the subject compounds include, but are not limited to, cancer of skeletal or smooth muscle, stomach cancer, cancer of the small intestine, rectum carcinoma, cancer of the salivary gland, endometrial cancer, adrenal cancer, anal cancer, rectal cancer, parathyroid cancer, and pituitary cancer.

Additional cancers that the compounds, combinations, and compositions described herein may be useful in preventing, treating and studying are, for example, colon carcinoma, familiary adenomatous polyposis carcinoma and hereditary non-polyposis colorectal cancer, or melanoma. Further, cancers include, but are not limited to, labial carcinoma, larynx carcinoma, hypopharynx carcinoma, tongue carcinoma, salivary gland carcinoma, gastric carcinoma, adenocarcinoma, thyroid cancer (medullary and papillary thyroid carcinoma), renal carcinoma, kidney parenchyma carcinoma, cervix carcinoma, uterine corpus carcinoma, endometrium carcinoma, chorion carcinoma, testis carcinoma, urinary carcinoma, melanoma, brain tumors such as glioblastoma, astrocytoma, meningioma, medulloblastoma and peripheral neuroectodermal tumors, gall bladder carcinoma, bronchial carcinoma, multiple myeloma, basalioma, teratoma, retinoblastoma, choroidea melanoma, seminoma, rhabdomyosarcoma, craniopharyngeoma, osteosarcoma, chondrosarcoma, myosarcoma, liposarcoma, fibrosarcoma, Ewing sarcoma, and plasmocytoma. In one aspect of the application, the present application provides for the use of the compounds, combinations, and compositions of the application in the manufacture of a medicament for the treatment of cancer, including without limitation the various types of cancer disclosed herein.

In some embodiments, the compounds, combinations, and compositions of this application are useful for treating cancer, such as colorectal, thyroid, breast, and lung cancer; and myeloproliferative disorders, such as polycythemia vera, thrombocythemia, myeloid metaplasia with myelofibrosis, chronic myelogenous leukemia, chronic myelomonocytic leukemia, hypereosinophilic syndrome, juvenile myelomonocytic leukemia, and systemic mast cell disease. In some embodiments, the compounds, combinations, and compositions of this application are useful for treating hematopoietic disorders, in particular, acute-myelogenous leukemia (AML), chronic-myelogenous leukemia (CML), acute-promyelocytic leukemia, and acute lymphocytic leukemia (ALL).

This application further embraces the treatment or prevention of cell proliferative disorders such as hyperplasias, dysplasias and pre-cancerous lesions. Dysplasia is the earliest form of pre-cancerous lesion recognizable in a biopsy by a pathologist. The subject compounds, combinations, and compositions may be administered for the purpose of preventing said hyperplasias, dysplasias or pre-cancerous lesions from continuing to expand or from becoming cancerous. Examples of pre-cancerous lesions may occur in skin, esophageal tissue, breast and cervical intra-epithelial tissue.

Examples of neurodegenerative diseases include, without limitation, Adrenoleukodystrophy (ALD), Alexander's disease, Alper's disease, Alzheimer's disease, Amyotrophic lateral sclerosis (Lou Gehrig's Disease), Ataxia telangiectasia, Batten disease (also known as Spielmeyer-Vogt-Sjogren-Batten disease), Bovine spongiform encephalopathy (BSE), Canavan disease, Cockayne syndrome, Corticobasal degeneration, Creutzfeldt-Jakob disease, Familial fatal insomnia, Frontotemporal lobar degeneration, Huntington's disease, HIV-associated dementia, Kennedy's disease, Krabbe's disease, Lewy body dementia, Neuroborreliosis, Machado-Joseph disease (Spinocerebellar ataxia type 3), Multiple System Atrophy, Multiple sclerosis, Narcolepsy, Niemann Pick disease, Parkinson's disease, Pelizaeus-Merzbacher Disease, Pick's disease, Primary lateral sclerosis, Prion diseases, Progressive Supranuclear Palsy, Refsum's disease, Sandhoff disease, Schilder's disease, Subacute combined degeneration of spinal cord secondary to Pernicious Anaemia, Spielmeyer-Vogt-Sjogren-Batten disease (also known as Batten disease), Spinocerebellar ataxia (multiple types with varying characteristics), Spinal muscular atrophy, Steele-Richardson-Olszewski disease, Tabes dorsalis, and Toxic encephalopathy.

Another aspect of this application provides a method for the treatment or lessening the severity of a disease selected from a proliferative or hyperproliterative disease, or a neurodegenerative disease, comprising administering an effective amount of a compound, combination, or composition of the application to a subject in need thereof. In other embodiments, the method further comprises administering a second agent that prevents EGFR dimer formation. In some embodiments, the second agent that prevents EGFR dimer formation is an antibody. In further embodiments, the second agent that prevents EGFR dimer formation is cetuximab, trastuzumab, or panitumumab. In further embodiments, the second agent that prevents EGFR dimer formation is cetuximab.

The compounds, combinations, and compositions of this application are also useful in biological samples. One aspect of the application relates to inhibiting protein kinase activity in a biological sample, which method comprises contacting said biological sample with a compound, combination, and composition of the application or a composition comprising the compound, combination, and composition. The term “biological sample”, as used herein, means an in vitro or an ex vivo sample, including, without limitation, cell cultures or extracts thereof; biopsied material obtained from a mammal or extracts thereof; and blood, saliva, urine, feces, semen, tears, or other body fluids or extracts thereof. Inhibition of protein kinase activity in a biological sample is useful for a variety of purposes that are known to one of skill in the art. Examples of such purposes include, but are not limited to, blood transfusion, organ-transplantation, and biological specimen storage.

Another aspect of this application relates to the study of kinases in biological and pathological phenomena; the study of intracellular signal transduction pathways mediated by such protein kinases; and the comparative evaluation of new protein kinase inhibitors. Examples of such uses include, but are not limited to, biological assays such as enzyme assays and cell-based assays.

The activity of the compounds, combinations, and compositions of the present application as kinase inhibitors may be assayed in vitro, in vivo, or in a cell line. In vitro assays include assays that determine inhibition of either the kinase activity or ATPase activity of the activated kinase. Alternate in vitro assays quantitate the ability of the inhibitor to bind to the protein kinase and may be measured either by radio labelling the inhibitor prior to binding, isolating the inhibitor/kinase complex and determining the amount of radio label bound, or by running a competition experiment where new inhibitors are incubated with the kinase bound to known radioligands. Detailed conditions for assaying a compound, combination, and composition utilized in this application as an inhibitor of various kinases are set forth in the Examples below.

### Pharmaceutical Compositions

In another aspect, the application provides a pharmaceutical composition comprising a pharmaceutical combination disclosed herein, together with a pharmaceutically acceptable carrier.

In another aspect, the application provides a pharmaceutical composition comprising a pharmaceutical combination disclosed herein, and a second agent that prevents EGFR dimer formation together with a pharmaceutically acceptable carrier. In some embodiments, the second agent that prevents EGFR dimer formation is an antibody. In further embodiments, the second agent that prevents EGFR dimer formation is cetuximab, trastuzumab, or panitumumab. In further embodiments, the second agent that prevents EGFR dimer formation is cetuximab.

Pharmaceutical combinations and compounds of the application can be administered as pharmaceutical compositions by any conventional route, in particular enterally, e.g., orally, e.g., in the form of tablets or capsules, or parenterally, e.g., in the form of injectable solutions or suspensions, topically, e.g., in the form of lotions, gels, ointments or creams, or in a nasal or suppository form. Pharmaceutical compositions comprising a pharmaceutical combination of the present application with at least one pharmaceutically acceptable carrier or diluent can be manufactured in a conventional manner by mixing, granulating or coating methods. For example, oral compositions can be tablets or gelatin capsules comprising the active ingredient together with a) diluents, e.g., lactose, dextrose, sucrose, mannitol, sorbitol, cellulose and/or glycine; b) lubricants, e.g., silica, talcum, stearic acid, its magnesium or calcium salt and/or polyethyleneglycol; for tablets also c) binders, e.g., magnesium aluminum silicate, starch paste, gelatin, tragacanth, methylcellulose, sodium carboxymethylcellulose and or polyvinylpyrrolidone; if desired d) disintegrants, e.g., starches, agar, alginic acid or its sodium salt, or effervescent mixtures; and/or e) absorbents, colorants, flavors and sweeteners. Injectable compositions can be aqueous isotonic solutions or suspensions, and suppositories can be prepared from fatty emulsions or suspensions. The compositions may be sterilized and/or contain adjuvants, such as preserving, stabilizing, wetting or emulsifying agents, solution promoters, salts for regulating the osmotic pressure and/or buffers. In addition, they may also contain other therapeutically valuable substances. Suitable formulations for transdermal applications include an effective amount of a compound or combination of the present application with a carrier. A carrier can include absorbable pharmacologically acceptable solvents to assist passage through the skin of the host. For example, transdermal devices are in the form of a bandage comprising a backing member, a reservoir containing the compound or combination optionally with carriers, optionally a rate controlling barrier to deliver the compound or combination to the skin of the host at a controlled and predetermined rate over a prolonged period of time, and means to secure the device to the skin. Matrix transdermal formulations may also be used. Suitable formulations for topical application, e.g., to the skin and eyes, are preferably aqueous solutions, ointments, creams or gels well-known in the art. Such may contain solubilizers, stabilizers, tonicity enhancing agents, buffers and preservatives.

Pharmaceutical combinations, compounds, and compositions of the application can be administered in therapeutically effective amounts in a combinational therapy with one or more therapeutic agents (pharmaceutical combinations) or modalities, e.g., a second agent that prevents EGFR dimer formation, non-drug therapies, etc. For example, synergistic effects can occur with agents that prevents EGFR dimer formation, other anti-proliferative, anti-cancer, immunomodulatory or anti-inflammatory substances. Where the pharmaceutical combinations, compounds, and compositions of the application are administered in conjunction with other therapies, dosages of the co-administered compounds will of course vary depending on the type of co-drug employed, on the specific drug employed, on the condition being treated and so forth.

Combination therapy includes the administration of the subject pharmaceutical combinations, compounds, and compositions in further combination with one or more other biologically active ingredients (such as, but not limited to, a second agent that prevents EGFR dimer formation, a second and different antineoplastic agent) and non-drug therapies (such as, but not limited to, surgery or radiation treatment). For instance, the pharmaceutical combinations, compounds, and compositions of the application can be used in combination with other pharmaceutically active compounds, preferably compounds that are able to enhance the effect of the combinations, compounds, and composition of the application. The pharmaceutical combinations, compounds, and compositions of the application can be administered simultaneously (as a single preparation or separate preparation) or sequentially to the other drug therapy or treatment modality. In general, a combination therapy envisions administration of two or more drugs during a single cycle or course of therapy.

In one aspect of the application, the pharmaceutical combinations, compounds, and compositions may be administered in combination with one or more agents that prevent EGFR dimer formation. In some embodiments, the second agent that prevents EGFR dimer formation is an antibody. In further embodiments, the second agent that prevents EGFR dimer formation is cetuximab, trastuzumab, or panitumumab. In further embodiments, the second agent that prevents EGFR dimer formation is cetuximab.

In another aspect of the application, the pharmaceutical combinations, compounds, and compositions may be administered in combination with one or more separate pharmaceutical agents, e.g., a chemotherapeutic agent, an immunotherapeutic agent, or an adjunctive therapeutic agent. In one embodiment, the chemotherapeutic agent reduces or inhibits the binding of ATP with EGFR (e.g., gefitinib, erlotinib, afatinib, lapatinib, nerabinib, CL-387785, AZD9291, CO-1686 or WZ4002).

The pharmaceutical compositions of the present application comprise a therapeutically effective amount of a pharmaceutical combination of the present application formulated together with one or more pharmaceutically acceptable carriers. As used herein, the term “pharmaceutically acceptable carrier” means a non-toxic, inert solid, semi-solid or liquid filler, diluent, encapsulating material or formulation auxiliary of any type. The pharmaceutical compositions of this application can be administered to humans and other animals orally, rectally, parenterally, intracisternally, intravaginally, intraperitoneally, topically (as by powders, ointments, or drops), buccally, or as an oral or nasal spray. In other embodiments, the composition further comprises a second agent that prevents EGFR dimer formation. In some embodiments, the second agent that prevents EGFR dimer formation is an antibody. In further embodiments, the second agent that prevents EGFR dimer formation is cetuximab, trastuzumab, or panitumumab. In further embodiments, the second agent that prevents EGFR dimer formation is cetuximab.

Liquid dosage forms for oral administration include pharmaceutically acceptable emulsions, microemulsions, solutions, suspensions, syrups and elixirs. In addition to the active component, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

In order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Compositions for rectal or vaginal administration are preferably suppositories which can be prepared by mixing the pharmaceutical combinations or compounds of this application with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid compositions of a similar type may also be employed as fillers in soft and hard filled gelatin capsules using such excipients as lactose or milk sugar as well as high molecular weight polyethylene glycols and the like.

The active components can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active component may be admixed with at least one inert diluent such as sucrose, lactose or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents.

Dosage forms for topical or transdermal administration of a pharmaceutical composition, compound, or composition of this application include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this application.

The ointments, pastes, creams and gels may contain, in addition to the active ingredient, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the active ingredient, excipients such as lactose, talc, silicic acid, aluminum hydroxide, calcium silicates and polyamide powder, or mixtures of these substances. Sprays can additionally contain customary propellants such as chlorofluorohydrocarbons.

Transdermal patches have the added advantage of providing controlled delivery of an active ingredient to the body. Such dosage forms can be made by dissolving or dispensing the active ingredient in the proper medium. Absorption enhancers can also be used to increase the flux of the pharmaceutical combinations or compounds across the skin. The rate can be controlled by either providing a rate controlling membrane or by dispersing the pharmaceutical combinations or compounds in a polymer matrix or gel.

The term “therapeutically effective amount”, as used herein, means a sufficient amount of pharmaceutical combinations, compounds, or compositions so as to decrease the symptoms of a disorder in a subject. As is well understood in the medical arts a therapeutically effective amount of pharmaceutical combinations, compounds, or compositions of this application will be at a reasonable benefit/risk ratio applicable to any medical treatment.

In general, pharmaceutical combinations, compounds, or compositions of the application will be administered in therapeutically effective amounts via any of the usual and acceptable modes known in the art, either singly or in combination with one or more therapeutic agents. A therapeutically effective amount may vary widely depending on the severity of the disease, the age and relative health of the subject, the potency of the compound used and other factors. In general, satisfactory results are indicated to be obtained systemically at daily dosages of from about 0.03 to 2.5 mg/kg per body weight. An indicated daily dosage in the larger mammal, e.g., humans, is in the range from about 0.5 mg to about 100 mg, conveniently administered, e.g., in divided doses up to four times a day or in retard form. Suitable unit dosage forms for oral administration comprise from ca. 1 to 50 mg active ingredient.

In certain embodiments, a therapeutic amount or dose of the pharmaceutical combinations, compounds, or compositions of the present application may range from about 0.1 mg/Kg to about 500 mg/Kg, alternatively from about 1 to about 50 mg/Kg. In general, treatment regimens according to the present application comprise administration to a patient in need of such treatment from about 10 mg to about 1000 mg of the pharmaceutical combinations, compounds, or compositions of this application per day in single or multiple doses. Therapeutic amounts or doses will also vary depending on route of administration, as well as the possibility of co-usage with other agents.

Upon improvement of a subject's condition, a maintenance dose of pharmaceutical combinations, compounds, or compositions of this application may be administered, if necessary. Subsequently, the dosage or frequency of administration, or both, may be reduced, as a function of the symptoms, to a level at which the improved condition is retained when the symptoms have been alleviated to the desired level, treatment should cease. The subject may, however, require intermittent treatment on a long-term basis upon any recurrence of disease symptoms.

It will be understood, however, that the total daily usage of the pharmaceutical combinations, compounds, or compositions of the present application will be decided by the attending physician within the scope of sound medical judgment. The specific inhibitory dose for any particular patient will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific active ingredients employed; and like factors well known in the medical arts.

The terms “co-administration” or “combined administration” or the like as utilized herein are meant to encompass administration of the selected therapeutic agents to a single patient, and are intended to include treatment regimens in which the agents are not necessarily administered by the same route of administration or at the same time.

The term “pharmaceutical combination” as used herein means a product that results from the mixing or combining of more than one active ingredient and includes both fixed and non-fixed combinations of the active ingredients. The term “fixed combination” means that the active ingredients, e.g., an allosteric EGFR inhibitor, and a co-agent, e.g., an ATP-competitive EGFR inhibitor, are both administered to a patient simultaneously in the form of a single entity or dosage. The term “non-fixed combination” means that the active ingredients, e.g., an allosteric EGFR inhibitor, and a co-agent, e.g., an ATP-competitive EGFR inhibitor, are both administered to a patient as separate entities either simultaneously, concurrently or sequentially with no specific time limits, wherein such administration provides therapeutically effective levels of the two active ingredients in the body of the patient. The latter also applies to cocktail therapy, e.g., the administration of three or more active ingredients.

Some examples of materials which can serve as pharmaceutically acceptable carriers include, but are not limited to, ion exchangers, alumina, aluminum stearate, lecithin, serum proteins, such as human serum albumin, buffer substances such as phosphates, glycine, sorbic acid, or potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, polyacrylates, waxes, polyethylenepolyoxypropylene-block polymers, wool fat, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes, oils such as peanut oil, cottonseed oil; safflower oil; sesame oil; olive oil; corn oil and soybean oil; glycols; such a propylene glycol or polyethylene glycol; esters such as ethyl oleate and ethyl laurate, agar; buffering agents such as magnesium hydroxide and aluminum hydroxide; alginic acid; pyrogen-free water, isotonic saline; Ringer's solution; ethyl alcohol, and phosphate buffer solutions, as well as other non-toxic compatible lubricants such as sodium lauyl sulfate and magnesium stearate, as well as coloring agents, releasing agents, coating agents, sweetening, flavoring and perfuming agents, preservatives and antioxidants can also be present in the composition, according to the judgment of the formulator. The protein kinase inhibitors or pharmaceutical salts thereof may be formulated into pharmaceutical compositions for administration to animals or humans. These pharmaceutical compositions, which comprise an amount of the protein inhibitor effective to treat or prevent a protein kinase-mediated condition and a pharmaceutically acceptable carrier, are other embodiments of the present application.

The application is further illustrated by the following examples and synthesis schemes, which are not to be construed as limiting this application in scope or spirit to the specific procedures herein described. It is to be understood that the examples are provided to illustrate certain embodiments and that no limitation to the scope of the application is intended thereby. It is to be further understood that resort may be had to various other embodiments, modifications, and equivalents thereof which may suggest themselves to those skilled in the art without departing from the spirit of the present application and/or scope of the appended claims.

### EXAMPLES

### Analytical Methods, Materials, and Instrumentation

Starting materials, reagents and solvents were purchased from commercial suppliers and were used without further purification unless otherwise noted. All reactions were monitored using a Waters Acquity UPLC/MS system (Waters PDA eλ Detector, QDa Detector, Sample manager-FL, Binary Solvent Manager) using Acquity UPLC® BEH C18 column (2.1×50 mm, 1.7 μm particle size): solvent gradient=85% A at 0 min, 1% A at 1.6 min; solvent A=0.1% formic acid in Water; solvent B=0.1% formic acid in Acetonitrile; flow rate: 0.6 mL/min. Reaction products were purified by flash column chromatography using CombiFlash®Rf with Teledyne Isco RediSep®R_{f} columns (4 g, 12 g, 24 g, 40 g, or 80 g) and Waters HPLC system using SunFire™ Prep C18 column (19×100 mm, 5 μm particle size): solvent gradient=80% A at 0 min, 10% A at 25 min; solvent A=0.035% TFA in Water; solvent B=0.035% TFA in MeOH: flow rate: 25 mL/min. ¹H NMR spectra were recorded on 500 MHz Bruker Avance III spectrometers. Chemical shifts are reported in parts per million (ppm, δ) downfield from tetramethylsilane (TMS). Coupling constants (J) are reported in Hz. Spin multiplicities are described as br (broad), s (singlet), d (doublet), t (triplet), q (quartet) and m (multiplet).

Abbreviations used in the following examples and elsewhere herein are:
atm atmosphere
br broad
DIPEA N,N-diisopropylethylamine
DMA N,N-dimethylacetamide
DMF N,N-dimethylformamide
DMSO dimethyl sulfoxide
ESI electrospray ionization
EtOAc ethyl acetate
HCl hydrochloric acid
h hour(s)
HATU bis(dimethylamino)methylene]-1H-1,2,3-triazolo[4,5-b]pyridinium 3-oxide hexafluoro-phosphate
HPLC high-performance liquid chromatography
LCMS liquid chromatography-mass spectrometry
m multiplet
MeOH methanol
MHz megahertz
min minutes
MS mass spectrometry
NMR nuclear magnetic resonance
Pd₂(dba)₃ tris(dibenzylideneacetone)dipalladium(0)
ppm parts per million
THF tetrahydrofuran
TLC thin layer chromatography
Xphos 2-dicyclohexylphosphino-2′,4′,6′-triisopropylbiphenyl

### Example 1: Synthesis of Intermediate I

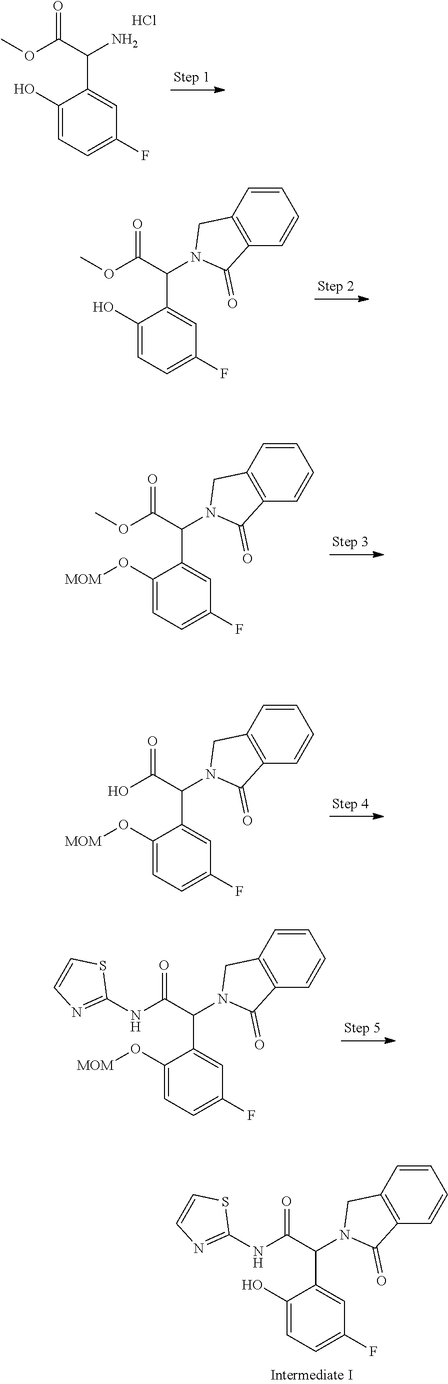

### Step 1. Methyl 2-(5-fluoro-2-hydroxyphenyl)-2-(1-oxoisoindolin-2-yl)acetate

To a solution of methyl 2-amino-2-(5-fluoro-2-hydroxyphenyl)acetate hydrochloride (250 mg, 1.06 mmol) and methyl 2-(bromomethyl)benzoate (243 mg, 1.06 mmol) in N,N-dimethylformamide (10 mL) was added DIEA (0.55 ml, 3.18 mmol) at 0° C. After stirring for 12 hours at 80° C., the reaction mixture was cooled to room temperature and diluted with ethyl acetate. The resulting solution was washed with water five times and washed with brine. The organic layer was dried over sodium sulfate, filtered and concentrated. The residue was purified by flash column chromatography (0 to 20% methanol in DCM) to provide methyl 2-(5-fluoro-2-hydroxyphenyl)-2-(1-oxoisoindolin-2-yl)acetate (200 mg, 60%) as a brown solid.

### Step 2. Methyl 2-(5-fluoro-2-(methoxymethoxy)phenyl)-2-(1-oxoisoindolin-2-yl)acetate

To a solution of methyl 2-(5-fluoro-2-hydroxyphenyl)-2-(1-oxoisoindolin-2-yl)acetate (20 mg, 0.063 mmol) in anhydrous CH₂Cl₂ (1 ml) were added DIEA (27 μl, 0.158 mmol) and MOMCl (10 μl, 0.127 mmol) at 0° C. After stirring for 2 hours, the reaction mixture was diluted with and washed with water and brine. The organic layer was dried over Na₂SO₄, filtered and concentrated under reduced pressure. The residue was purified by flash column chromatography (hexane:EtOAc=80:20 to 30:70) to obtain Methyl 2-(5-fluoro-2-(methoxymethoxy)phenyl)-2-(1-oxoisoindolin-2-yl)acetate as an off-white solid (19 mg, 82%).

### Step 3. 2-(5-fluoro-2-hydroxyphenyl)-2-(1-oxoisoindolin-2-yl)acetic acid

To a solution of methyl 2-(5-fluoro-2-hydroxyphenyl)-2-(1-oxoisoindolin-2-yl)acetate (50 mg, 0.139 mmol) in THF/MeOH/water was added lithium hydroxide monohydrate (29 mg, 0.696 mmol). After stirring for 1 hour, the solvent was removed under reduced pressure and the resulting residue was diluted with ice water. The aqueous mixture was acidified with concentrated HCl and the resulting suspension isolated via filtration. The solid was dried using a stream of nitrogen gas to provide 2-(5-fluoro-2-hydroxyphenyl)-2-(1-oxoisoindolin-2-yl)acetic acid (48 mg, quantitative).

### Step 4. 2-(5-fluoro-2-(methoxymethoxy)phenyl)-2-(1-oxoisoindolin-2-yl)-N-(thiazol-2-yl)acetamide

To a solution of 2-(5-fluoro-2-hydroxyphenyl)-2-(1-oxoisoindolin-2-yl)acetic acid (80 mg, 0.463 mmol), thiazol-2-amine (46 mg, 0.463 mmol), and HATU (220 mg, 0.580 mmol) in N,N-dimethylformamide (1.5 ml) was added DIPEA (0.16 ml, 0.926 mmol). After stirring for 6 hours, the reaction mixture was diluted with EtOAc and washed with water five times. The organic layer was dried over sodium sulfate, filtered, concentrated under reduced pressure, and purified by preparative high performance liquid chromatography (HPLC) to obtain. 2-(5-fluoro-2-(methoxymethoxy)phenyl)-2-(1-oxoisoindolin-2-yl)-N-(thiazol-2-yl)acetamide (71 mg, 72%).

### Step 5. 2-(5-fluoro-2-hydroxyphenyl)-2-(1-oxoisoindolin-2-yl)-N-(thiazol-2-yl)acetamide

To a solution of 2-(5-fluoro-2-(methoxymethoxy)phenyl)-2-(1-oxoisoindolin-2-yl)-N-(thiazol-2-yl)acetamide (80 mg, 0.187 mmol) in CH₂Cl₂ (1.6 ml) was added trifluoroacetic acid (0.4 ml) at 0° C. After stirring for 4 hours, the reaction mixture was diluted with CH₂Cl₂ and extracted with sat. NaHCO₃. The aqueous layer was further basified with sat. NaHCO₃ and extracted with CH₂Cl₂ three times. Then, the combined organic layer was washed with brine, dried over sodium sulfate, filtered and concentrated under reduced pressure. The residue was purified by prepHPLC to give 2-(5-fluoro-2-hydroxyphenyl)-2-(1-oxoisoindolin-2-yl)-N-(thiazol-2-yl)acetamide (39 mg, 55%) as a white solid.

### Example 2: Synthesis of Compound A

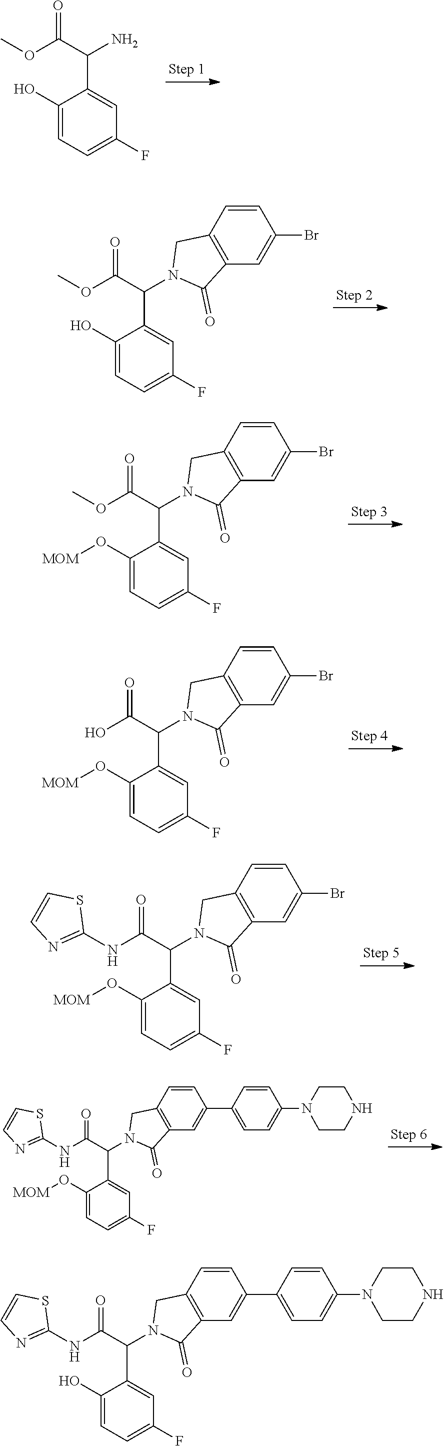

### Step 1. Methyl 2-(5-bromo-1-oxoisoindolin-2-yl)-2-(5-fluoro-2-hydroxyphenyl)acetate

To a solution of methyl 2-amino-2-(5-fluoro-2-hydroxyphenyl)acetate hydrochloride (2.80 g, 13.92 mmol) and methyl 5-bromo-2-(bromomethyl)benzoate (3.90 g, 12.66 mmol) in N,N-dimethylformamide (120 mL) was added DIEA (6.60 mL, 37.98 mmol) at 0° C. After stirring for 12 hours at 80° C., the reaction mixture was cooled to room temperature and diluted with ethyl acetate. The resulting solution was washed with water five times and washed with brine. The organic layer was dried over sodium sulfate, filtered and concentrated. The residue was purified by flash column chromatography (0 to 20% methanol in DCM) to give methyl 2-(5-bromo-1-oxoisoindolin-2-yl)-2-(5-fluoro-2-hydroxyphenyl)acetate (3.28 g, 72%) as a brown solid.

### Step 2. Methyl 2-(6-bromo-1-oxoisoindolin-2-yl)-2-(5-fluoro-2-(methoxymethoxy)phenyl)acetate

To a solution of Methyl 2-(5-bromo-1-oxoisoindolin-2-yl)-2-(5-fluoro-2-hydroxyphenyl)acetate (350 mg, 0.90 mmol) and DIEA (0.47 mL, 2.70 mmol) in DCM (5 mL) was added chloromethyl methyl ether (0.17 mL, 2.25 mmol) dropwise at 0° C. After stirring at 40° C. for 6 hours, the reaction mixture was diluted with DCM, washed with water and brine. The organic layer was dried over sodium sulfate, filtered and concentrated. The residue was purified by flash column chromatography (0 to 20% methanol in DCM) to provide methyl 2-(6-bromo-1-oxoisoindolin-2-yl)-2-(5-fluoro-2-(methoxymethoxy)phenyl)acetate (363 mg, 92%) as an off-white solid.

### Step 3. 2-(5-bromo-1-oxoisoindolin-2-yl)-2-(5-fluoro-2-hydroxyphenyl)acetic acid

2-(5-bromo-1-oxoisoindolin-2-yl)-2-(5-fluoro-2-hydroxyphenyl)acetic acid was prepared by the procedure that used to synthesize 2-(5-fluoro-2-hydroxyphenyl)-2-(1-oxoisoindolin-2-yl)acetic acid.

### Step 4. 2-(6-bromo-1-oxoisoindolin-2-yl)-2-(5-fluoro-2-(methoxymethoxy)phenyl)-N-(thiazol-2-yl)acetamide

2-(6-bromo-1-oxoisoindolin-2-yl)-2-(5-fluoro-2-(methoxymethoxy)phenyl)-N-(thiazol-2-yl)acetamide was prepared by the procedure that used to synthesize 2-(5-fluoro-2-(methoxymethoxy)phenyl)-2-(1-oxoisoindolin-2-yl)-N-(thiazol-2-yl)acetamide.

### Step 5. 2-(5-fluoro-2-(methoxymethoxy)phenyl)-2-(1-oxo-6-(4-(piperazin-1-yl)phenyl)isoindolin-2-yl)-N-(thiazol-2-yl)acetamide

After degassing by sonication for 10 seconds, a mixture of 2-(6-bromo-1-oxoisoindolin-2-yl)-2-(5-fluoro-2-(methoxymethoxy)phenyl)-N-(thiazol-2-yl)acetamide (2.00 g, 3.95 mmol), 1-(4-(4,4,5,5-tetramethyl-1,3,2-dioxaborolan-2-yl)phenyl)piperazine hydrochloride (1.54 g, 4.74 mmol) and 2 N sodium carbonate (5.9 mL, 11.9 mmol) in dioxane (13 mL) was preheated at 100° C. for 20 min. Then, PdCl₂(dppf)₂(293 mg, 0.40 mmol) and Xphos (286 mg, 0.60 mmol) were added carefully to the reaction mixture. After stirring at 100° C. for 8 hours, the reaction mixture was cooled to room temperature, filtered through a pad of celite. The filtrate was diluted with DCM and washed with water and brine. The organic layer was dried over sodium sulfate, filtered and concentrated under reduced pressure. The residue was purified by flash chromatography DCM:MeOH=100:0 to 80:20) to obtain 2-(5-fluoro-2-(methoxymethoxy)phenyl)-2-(1-oxo-6-(4-(piperazin-1-yl)phenyl)isoindolin-2-yl)-N-(thiazol-2-yl)acetamide as a brown solid (1.69 g, 73%).

### Step 6. 2-(5-fluoro-2-hydroxyphenyl)-2-(1-oxo-6-(4-(piperazin-1-yl)phenyl)isoindolin-2-yl)-N-(thiazol-2-yl)acetamide

2-(5-fluoro-2-hydroxyphenyl)-2-(1-oxo-6-(4-(piperazin-1-yl)phenyl)isoindolin-2-yl)-N-(thiazol-2-yl)acetamide was synthesized using analog procedure that used to make 2-(5-fluoro-2-hydroxyphenyl)-2-(1-oxoisoindolin-2-yl)-N-(thiazol-2-yl)acetamide (Compound A). MS m/z: 543.89 [M+1]⁺. ¹H NMR (500 MHz, DMSO-d₆) δ 12.62 (br s, NH), 10.00 (br s, OH), 7.90-7.86 (m, 3H), 7.69-7.65 (m, 2H), 7.62 (d, J=7.9 Hz, 1H), 7.49 (d, J=3.4 Hz, 1H), 7.28 (d, J=3.4 Hz, 1H), 7.15-7.09 (m, 3H), 6.94 (dd, J=4.9, 8.9 Hz, 1H), 6.87 (dd, J=3.1, 9.2 Hz, 1H), 6.34 (s, 1H), 4.64 (d, J=17.7 Hz, 1H), 4.02 (d, J=17.7 Hz, 1H), 3.46-3.42 (m, 4H), 3.27-3.23 (m, 4H), 2.56 (br s, NH).

### Example 3: Synthesis of Biotin Conjugate of Compound A

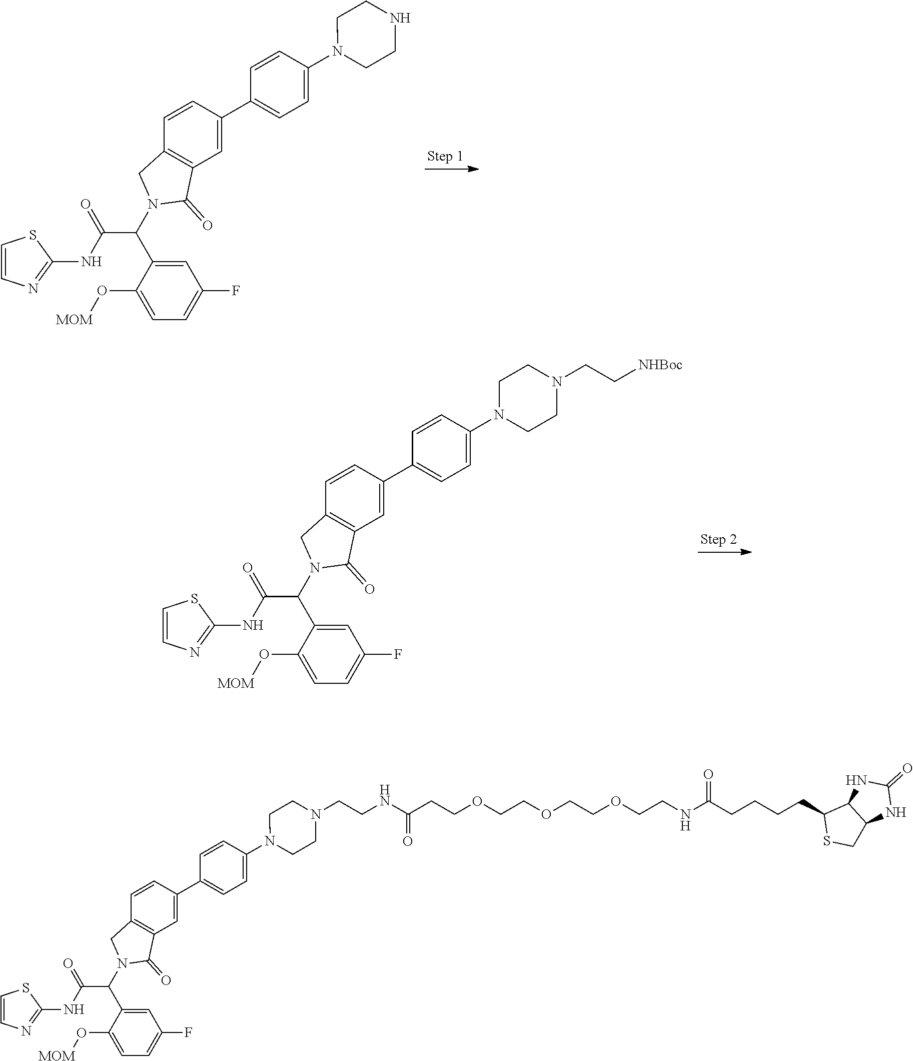

### Step 1. tert-Butyl (2-(4-(4-(2-(1-(5-fluoro-2-(methoxymethoxy)phenyl)-2-oxo-2-(thiazol-2-ylamino)ethyl)-3-oxoisoindolin-5-yl)phenyl)piperazin-1-yl)ethyl)carbamate

To a solution of 2-(5-fluoro-2-(ethoxymethoxy)phenyl)-2-(1-oxo-6-(4-(piperazin-1-yl)phenyl)isoindolin-2-yl)-N-(thiazol-2-yl)acetamide (90 mg, 0.15 mmol) and tert-butyl (2-bromoethyl)carbamate (34 mg, 0.15 mmol) in dioxane (2 mL) was added DIEA (53 μl, 0.31 mmol). After stirring at 80° C. for 12 hours, the resulting mixture was diluted with DMSO and purified by preparative HPLC to obtain tert-Butyl (2-(4-(4-(2-(1-(5-fluoro-2-(methoxymethoxy)phenyl)-2-oxo-2-(thiazol-2-ylamino)ethyl)-3-oxoisoindolin-5-yl)phenyl)piperazin-1-yl)ethyl)carbamate (87 mg, 78%) as an off-white solid. LC/MS (ESI) m/z 731.84 [M+H]⁺.

### Step 2. Compound A-biotin conjugate (N-(15-(4-(4-(2-(1-(5-fluoro-2-hydroxyphenyl)-2-oxo-2-(thiazol-2-ylamino)ethyl)-3-oxoisoindolin-5-yl)phenyl)piperazin-1-yl)-12-oxo-3,6,9-trioxa-13-azapentadecyl)-5-((3aS,4S,6aR)-2-oxohexahydro-1H-thieno[3,4-d]imidazol-4-yl)pentanamide)

To a solution of tert-Butyl (2-(4-(4-(2-(1-(5-fluoro-2-(methoxymethoxy)phenyl)-2-oxo-2-(thiazol-2-ylamino)ethyl)-3-oxoisoindolin-5-yl)phenyl)piperazin-1-yl)ethyl)carbamate (87 mg, 0.12 mmol) in dioxane (1 mL) was added a 4 M solution of hydrochloric acid in dioxane (3 mL). After stirring for 6 hours, the reaction mixture was concentrated under reduced pressure to afford 2-(6-(4-(4-(2-aminoethyl)piperazin-1-yl)phenyl)-1-oxoisoindolin-2-yl)-2-(5-fluoro-2-(methoxymethoxy)phenyl)-N-(thiazol-2-yl)acetamide hydrogen chloride which was used to next step without further purification. LC/MS (ESI) m-z 587.66 [M+H]⁺.

The crude compound was dissolved in N,N-dimethylformamide (1.5 mL) and the resulting mixture was cooled to 0° C. DIEA (0.06 mL, 0.36 mmol) was carefully added to the mixture followed by adding 2,5-dioxopyrrolidin-1-yl 14-oxo-18-((3aS,4S,6aR)-2-oxohexahydro-1H-thieno[3,4-d]imidazol-4-yl)-4,7,10-trioxa-13-azaoctadecanoate (62 mg, 0.11 mmol). After stirring for 30 min, the reaction mixture was diluted with DMSO and purified by preparative HPLC to give Compound A-biotin conjugate (29 mg, 25%, two steps) as an off-white solid. ¹H NMR (500 MHz, DMSO-d₆) δ 12.61 (br s, 1H), 10.03 (s, 1H), 8.22 (br s, 1H), 7.89 (s, 1H), 7.87 (dd, J=7.9, 1.5 Hz, 1H), 7.83 (t, J=5.6 Hz, 1H), 7.66 (d, J=8.9 Hz, 2H), 7.61 (d, J=7.9 Hz, 1H), 7.49 (d, J=3.7 Hz, 1H), 7.27 (d, J=3.7 Hz, 1H), 7.14-7.09 (m, 3H), 6.93 (dd, J=8.9, 4.9 Hz, 1H), 6.86 (dd, J=9.2, 3.4 Hz, 1H), 6.41 (s, 1H), 6.36 (s, 1H), 6.33 (s, 1H), 4.63 (d, J=17.7 Hz, 1H), 4.30 (dd, J=7.6, 5.2 Hz, 1H), 4.15-4.09 (m, 1H), 4.01 (d, J=17.7 Hz, 1H), 3.92 (br s, 2H), 3.62 (t, J=6.4 Hz, 2H), 3.55-3.32 (m, 14H), 3.39 (t, J=6.0 Hz, 2H), 3.27-3.13 (m, 4H), 3.18 (q, J=5.8 Hz, 2H), 3.11-3.04 (m, 1H), 2.81 (dd, J=12.5, 5.2 Hz, 1H), 2.57 (d, J=12.2 Hz, 1H), 2.37 (t, J=6.4 Hz, 2H), 2.06 (t, J=7.3 Hz, 2H), 1.66-1.55 (m, 1H), 1.55-1.39 (m, 3H), 1.36-1.20 (m, 2H); LC/MS (ESI) m/z 1016.58 [M+H]⁺.

### Example 4: Biological/Biochemical Studies

### Cell Viability Assays

H3255GR cells were treated with increasing concentrations of inhibitors for 72 hours and growth or the inhibition of growth was assessed by MTS assay according to previously established methods (Engelman et al., 2006; Ercan et al., 2015; Zhou et al., 2009). All experimental points were set up in six technical replicates and all experiments were repeated at least three times.

### Western Blotting

To assess the effect of compounds on EGFR and its downstream pathways, NIH-3T3, H1975, H3255GR cells were treated for 4 hours before cells were lysed with NP40 lysis buffer, supplemented with protease and phosphatase inhibitors, followed by protein quantification. 20 μg of lysates were used for Western Blotting analyses. For experiments that examine the effect of Compound A in the presence of EGF, cells were treated with 10 ng/ml of EGF for 15 minutes before they were treated with drugs for 4 hours followed by lysis and protein quantification as described above. All experiments were done at least three times.

### Biotinylated Drug Pull Down Assay

For in vitro pull down assays, cells were treated with dose-escalated WZ-4002, Compound O for two hours before they were subjected to lysis and protein quantification. 15-20 μg of proteins lysates were aliquoted and loaded at the same time as the pull down assay to ensure the presence of EGFR protein, phospho-EGFR activity. Tubulin expression was assessed to ensure even loading of gels. 500 μg of protein was incubated with either biotinylated-linker (control) or with biotinylated Compound A for two hours before 50% NeutrAvidin agarose beads (Thermo Fisher Scientific) slurry was added for an hour to precipitate the EGFR that was associated to the biotinylated allosteric inhibitor. The beads were then washed three times with PBS containing 1% IGEPAL and an insulin syringe was used to remove extraneous buffer before the samples were suspended in 2×SDS sample preparation buffer for Western blotting analyses. All experiments were performed at least three times.

### ENU Mutagenesis

N-ethyl-N-nitrosourea (ENU) was purchased from Sigma Aldrich and mutagenesis studies were carried as previously described (Ercan et al., 2015). Briefly, 1×10⁶ cells/ml of L858R and L858R/T790M Ba/F3 cells were treated with 50 μg/ml of ENU for 24 hours before the cells were washed three times in RPMI media and expanded for 3 days. 1×10⁴ cells per well were plated in 96 wells and 5 plates were plated per condition. These cells were treated continuously with either DMSO, 1 μM gefitinib, 1 μM Compound 0, 10 μM Compound A alone or with gefitinib/Compound A or Compound O/Compound A drug combinations for 4 weeks with media and drug change once a week. Cell growth was monitored and number of resistant clones were counted and expanded.

### IncuCyte Studies

For cell confluency studies, H3255GR cells were treated with different inhibitors and monitored by the automated microscopy using the IncuCyte Live-Cell Imaging system (Essen Bioscience). Confluency was measured by averaging the percentage of area that the cells occupied from three images of a given well every two hours for 72 hours. For apoptosis studies, cells were treated with inhibitors incubated in media containing the CellEvent™ Caspase 3/7 Green ReadyProbes® reagent (Thermo Fisher Scientific) and monitored for change in green fluorescence activity using the aforementioned imaging system. The average number of objects that were stained with green from three images per well was counted as positive for Caspase 3/7, indicating apoptosis, and recorded every two hours for 72 hours. All experimental conditions were set up in at least six replicates and all experiments were performed at least three times.

### In Vivo Studies

All breeding, mouse husbandry, and in vivo experiments were performed with the approval of the Dana-Farber Cancer Institute (Boston, Mass.) Animal Care and Use Committee.

For the H1975 xenograft study, Nu/Nu mice were purchased from Charles River Laboratories International Inc. H1975 cells were detected as pathogen free at Charles River Laboratories International Inc. and were resuspended in serum-free medium mixed with an equal amount of Matrigel (BD Biosciences). Mice were injected at 2 locations per mouse in the flanks with 2 million cells per shot. The mice were randomly grouped, and treatment started when tumor size reached 100 to 200 mm³. Each cohort included at least 5 mice. Tumor sizes were monitored weekly, and volumes were calculated using the following formula: (mm³)=length×width×width 0.5.

To assess EGFR activity in the mice after the study was performed, tumors were taken 3 hours after the last dose for pharmacodynamic (PD) studies. Tumors were flash frozen in liquid nitrogen to preserve tissue integrity and homogenized in RIPA buffer supplemented with protease and phosphatase inhibitors. The protein was quantified and 20 μg of lysates were used for Western Blotting analyses.

In the H1975 xenograft study, Compound A was dissolved in 5% NMP (5% 1-methyl-2-pyrrolidinone: 95% PEG-300). Compound A was dosed at 100 mg/kg once daily orally. Compound O was dissolved in 0.5% HMPC (0.5% Hydroxypropyl methylcellulose: 99.5% 0.05N hydrogen chloride). Mice received 25 mg/kg Compound O once daily orally.

### EQUIVALENTS

Those skilled in the art will recognize, or be able to ascertain, using no more than routine experimentation, numerous equivalents to the specific embodiments described specifically herein. Such equivalents are intended to be encompassed in the scope of the following claims.

1. A pharmaceutical combination comprising an allosteric EGFR inhibitor and an ATP-competitive EGFR inhibitor, wherein:
the allosteric EGFR inhibitor is a compound of Formula I: 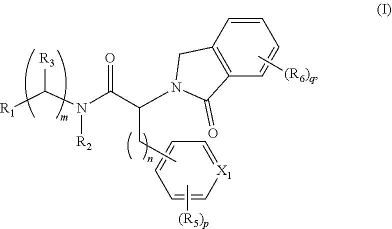
or a pharmaceutically acceptable salt, hydrate, or solvate thereof, and
the ATP-competitive EGFR inhibitor is a compound of Formula I′: 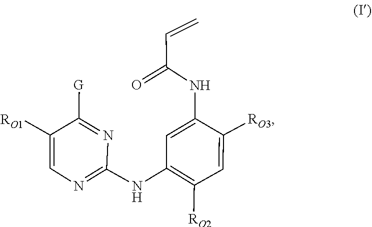
or a pharmaceutically acceptable salt, hydrate, or solvate thereof,
wherein:
R₁ is C₆-C₁₀ aryl, or heteroaryl comprising one or two 5- to 7-membered rings and 1-4 heteroatoms selected from N, O, and S, wherein the aryl or heteroaryl is optionally substituted with one or more R₁₁;
each R₁₁ is independently C₁-C₄ alkyl, C₁-C₄ haloalkyl, C₁-C₄ alkoxy, C₁-C₄ haloalkoxy, halogen, NO₂, OH, CN, C(O)R₁₃, C(O)OR₁₃, C(O)NR₁₃R₁₄, NR₁₃R₁₄, C₃-C₇ cycloalkyl, heterocyclyl comprising one 5- to 7-membered ring and 1-3 heteroatoms selected from N, O, and S, C₆-C₁₀ aryl, or heteroaryl comprising one or two 5- to 7-membered rings and 1-4 heteroatoms selected from N, O, and S, wherein the alkyl, cycloalkyl, heterocyclyl, aryl, or heteroaryl is optionally substituted with one or more R₁₂;
each R₁₂ is independently C₁-C₄ alkyl, C₁-C₄ haloalkyl, C₁-C₄ alkoxy, C₁-C₄ haloalkoxy, halogen, NO₂, OH, CN, C₃-C₇ cycloalkyl, heterocyclyl comprising one 5- to 7-membered ring and 1-3 heteroatoms selected from N, O, and S, C₆-C₁₀ aryl, or heteroaryl comprising one or two 5- to 7-membered rings and 1-4 heteroatoms selected from N, O, and S, wherein the aryl or heteroaryl is optionally substituted with one or more substituents independently selected from C₁-C₄ alkyl, C₁-C₄ alkoxy, C₁-C₄ haloalkyl, C₁-C₄ haloalkoxy, halogen, NH₂, NH(C₁-C₄) alkyl, N((C₁-C₄) alkyl)₂, C₃-C₇ cycloalkyl, and heterocyclyl comprising one 5- to 7-membered ring and 1-3 heteroatoms selected from N, O, and S;
each R₁₃ is independently 1, C₁-C₄ alkyl, C₃-C₇ cycloalkyl, or heterocyclyl comprising one 5- to 7-membered ring and 1-3 heteroatoms selected from N, O, and S, wherein the alkyl, cycloalkyl, or heterocyclyl is optionally substituted with one or more substituents independently selected from C₁-C₄ alkyl, halogen, OH, NH₂, NH(C₁-C₄) alkyl, N((C₁-C₄) alkyl)₂, and heterocyclyl comprising one 5- to 7-membered ring and 1-3 heteroatoms selected from N, O, and S;
each R₁₄ is independently H or C₁-C₃ alkyl;
R₂ is H or C₁-C₃ alkyl;
R₃ is H or C₁-C₃ alkyl;
X₁ is N or CR₄;
R₄ is H, C₁-C₄ alkyl, C₁-C₄ haloalkyl, C₁-C₄ alkoxy, C₁-C₄ haloalkoxy, halogen, NO₂, NH₂, OH, or CN;
each R₅ is independently C₁-C₄ alkyl, C₁-C₄ haloalkyl, C₁-C₄ alkoxy, C₁-C₄ haloalkoxy, halogen, NO₂, NH₂, OH, or CN;
each R₆ is independently halogen, C₃-C₇ cycloalkyl, C₄-C₇ cycloalkenyl, C₆-C₁₀ aryl, NH—(C₆-C₁₀) aryl, or heteroaryl comprising one or two 5- to 7-membered rings and 1-4 heteroatoms selected from N, O, and S, wherein the aryl or heteroaryl is optionally substituted with one or more R₇;
each R₇ is independently C₁-C₄ alkyl, C₁-C₄ alkoxy, C₁-C₄ haloalkyl, C₁-C₄ haloalkoxy, halogen, C(O)OH, C(O)O(C₁-C₄) alkyl, C(O)NR₈R₉, NF₂, OH, CN, O(CH₂)₀₋₃—(C₆-C₁₀) aryl, or (CH₂)₀₋₃-heterocyclyl which comprises one 5- to 7-membered ring and 1-3 heteroatoms selected from N, O, and S, wherein the heterocyclyl is optionally substituted with one or more substituents independently selected from C₁-C₄ alkyl, C₁-C₄ alkoxy, C₁-C₄ haloalkyl, C₁-C₄ haloalkoxy, halogen, and C(O)O((C₁-C₄) alkyl);
R₈ is H or C₁-C₃ alkyl;
R₉ is H or C₁-C₄ alkyl optionally substituted with one or more substituents independently selected from NH₂, NH(C₁-C₄) alkyl, N((C₁-C₄) alkyl)₂, and heterocyclyl comprising one 5- to 7-membered ring and 1-3 heteroatoms selected from N, O, and S; or
R₈ and R₉ together with the nitrogen atom to which they are attached form a 5- or 6-membered heterocyclyl optionally containing 1-2 additional heteroatoms selected from N O, and S;
in and n are each independently 0 or 1;
q is 0, 1, or 2; and
p is 0, 1, 2, 3 or 4,
provided that when m is 0, n is 0, p is 0, q is 0, and X₁ is CH, then R₁ is not 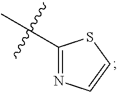 [SMILES: *c1nccs1]
and
that when p is 2, X₁ is CH, and one R₅ is 4-fluoro, then the other R₅ is not 2-hydroxy.
G is 4,5,6,7-tetrahydropyrazolo[1,5-a]pyridin-3-yl, 1H-indol-3-yl, 1-methyl-1H-indol-3-yl, or pyrazolo[1,5-a]pyridin-3-yl, and
R_{O1} is H, F, Cl, methyl, or CN;
R_{O2} is methoxy or methyl; and
R_{O3} is (3R)-3-(dimethylamino)pyrrolidin-1-yl, (3S)-3-(dimethylamino)pyrrolidin-1-yl, 3-(dimethylamino)azetidin-1-yl, (2-(dimethylamino)ethyl)-methylamino, (2-(methylamino)ethyl)-methylamino, 5-methyl-2,5-diazaspiro[3.4]oct-2-yl, (3aR,6aR)-5-methylhexahydro-pyrrolo[3,4-b]pyrrol-1(2H)-yl, 1-methyl-1,2,3,6-tetrahydropyridin-4-yl, 4-methylpiperazin-1-yl, 4-(2-(dimethylamino)-2-oxoethyl)piperazin-1-yl, methyl(2-(4-methylpiperazin-1-yl)ethyl)amino, methyl(2-(morpholin-4-yl)ethyl)amino, 1-amino-1,2,3,6-tetrahydropyridin-4-yl, or 4-((2S)-2-aminopropanoyl)piperazin-1-yl.

2. The pharmaceutical combination of claim 1, wherein R₂ is H.

3. The pharmaceutical combination of claim 1, wherein R₂ is C₁-C₃ alkyl.

4. The pharmaceutical combination of any one of claims 1-3, wherein R₃ is H.

5. The pharmaceutical combination of any one of claims 1-3, wherein R₃ is C₁-C₃ alkyl.

6. The pharmaceutical combination of any one of claims wherein X₁ is N.

7. The pharmaceutical combination of any one of claims 1-5, wherein X₁ is CR₄.

8. The pharmaceutical combination of any one of claims 1-5 and 7, wherein R₄ is H.

9. The pharmaceutical combination of any one of claims 1-5 and 7, wherein R₄ is C₁-C₄ alkyl or C₁-C₄ haloalkyl.

10. The pharmaceutical combination of any one of claims 1-5 and 7, wherein R₄ is C₁-C₄ alkoxy or C₁-C₄ haloalkoxy.

11. The pharmaceutical combination of any one of claims 1-5 and 7, wherein R₄ is halogen.

12. The pharmaceutical combination of any one of claims 1-5 and 7, wherein R₄ is NO₂, NH₂, OH, or CN.

13. The pharmaceutical combination of any one of claims 1-12, wherein at least one R₅ is C₁-C₄ alkyl or C₁-C₄ haloalkyl.

14. The pharmaceutical combination of any one of claims 1-12, wherein at least one R₅ is C₁-C₄ alkoxy or C₁-C₄ haloalkoxy.

15. The pharmaceutical combination of any one of claims 1-12, wherein at least one R₅ is halogen.

16. The pharmaceutical combination of any one of claims 1-12, wherein at least one R₅ is NO₂, NH₂, OH, or CN.

17. The pharmaceutical combination of any one of claims 1-12, wherein at least one R₅ is halogen and at least one R₅ is OH.

18. The pharmaceutical combination of any one of claims 1-12, wherein one R₅ is halogen and one R₅ is OH.

19. The pharmaceutical combination of any one of claims 1-18, wherein at least one R₆ is halogen.

20. The pharmaceutical combination of any one of claims 1-18, wherein at least one R₆ is C₃-C₇ cycloalkyl.

21. The pharmaceutical combination of any one of claims 1-18, wherein at least one R₆ is C₄-C₇ cycloalkenyl.

22. The pharmaceutical combination of any one of claims 1-18, wherein at least one R₆ is C₆-C₁₀ aryl optionally substituted with one or more R₇.

23. The pharmaceutical combination of any one of claims 1-18, wherein at least one R₆ is NH—(C₆-C₁₀) aryl optionally substituted with one or more R₇.

24. The pharmaceutical combination of any one of claims 1-18, wherein at least one R₆ is heteroaryl comprising one or two 5- to 7-membered rings and 1-4 heteroatoms selected from N, O, and S optionally substituted with one or more R₇.

25. The pharmaceutical combination of any one of claims 1-24, wherein at least one R₇ is C₁-C₄ alkyl or C₁-C₄ haloalkyl.

26. The pharmaceutical combination of any one of claims 1-24, wherein at least one R₇ is C₁-C₄ alkoxy or C₁-C₄ haloalkoxy.

27. The pharmaceutical combination of any one of claims 1-24, wherein at least one R₇ is halogen.

28. The pharmaceutical combination of any one of claims 1-24, wherein at least one R₇ is C(O)OH or C(O)O(C₁-C₄) alkyl.

29. The pharmaceutical combination of any one of claims 1-24, wherein at least one R₇ is NH₂, OH, or CN.

30. The pharmaceutical combination of any one of claims 1-24, wherein at least one R₇ is C(O)NR₈R₉.

31. The pharmaceutical combination of any one of claims 1-24, wherein at least one R₇ is O(CH₂)₀₋₃—(C₆-C₁₀) aryl.

32. The pharmaceutical combination of any one of claims 1-24, wherein at least one R₇ is (CH₂)₀₋₃-heterocyclyl which comprises one 5- to 7-membered ring and 1-3 heteroatoms selected from N, O, and S.

33. The pharmaceutical combination of any one of claims 1-32, wherein R₁ is C₆-C₁₀ aryl optionally substituted with one or more R₁₁.

34. The pharmaceutical combination of any one of claims 1-32, wherein R₁ is heteroaryl comprising one or two 5- to 7-membered rings and 1-4 heteroatoms selected from N, O, and S optionally substituted with one or more R₁₁.

35. The pharmaceutical combination of any one of claims 1-32, wherein R₁ is selected from: 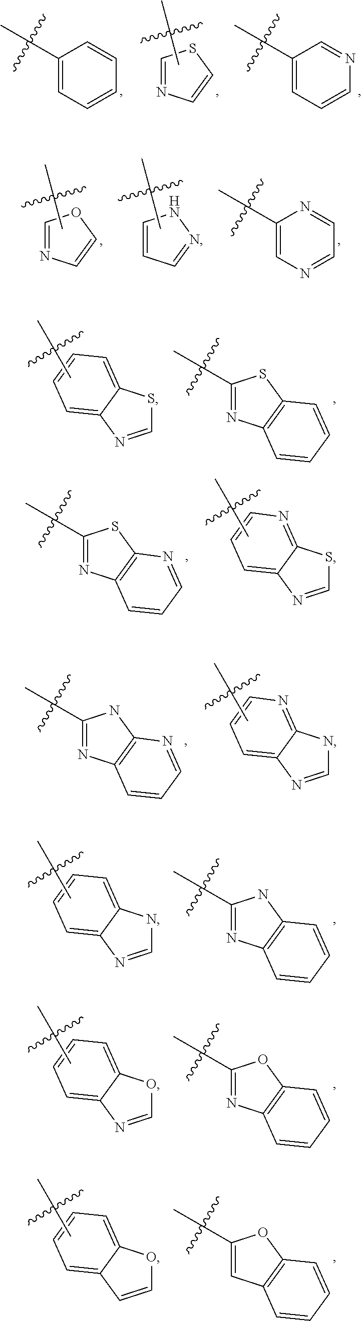 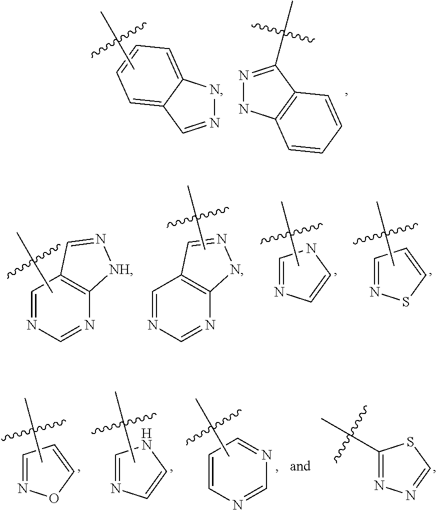
wherein each moiety is optionally substituted with one or more R₁₁.

36. The pharmaceutical combination of any one of claims 1-35, wherein m is 0.

37. The pharmaceutical combination of any one of claims 1-35, wherein m is 1.

38. The pharmaceutical combination of any one of claims 1-37, wherein n is 0.

39. The pharmaceutical combination of any one of claims 1-37, wherein n is 1.

40. The pharmaceutical combination of claim 1, wherein the allosteric EGFR inhibitor is a compound of Formula II or III: 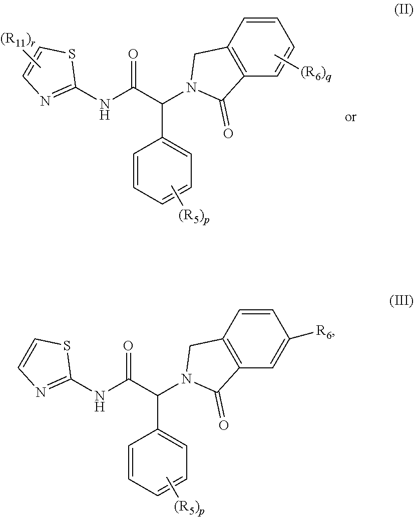
or a pharmaceutically acceptable salt, hydrate, or solvate thereof, wherein r is 0, 1, or 2.

41. The pharmaceutical combination of claim 1, wherein the allosteric EGFR inhibitor is Compound A: 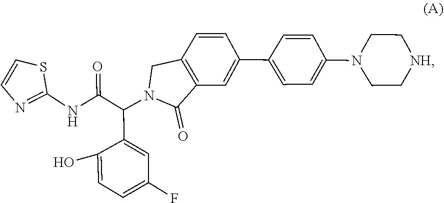 [SMILES: O=C(Nc1nccs1)C(c1cc(F)ccc1O)N1Cc2ccc(-c3ccc(N4CCNCC4)cc3)cc2C1=O]
or a pharmaceutically acceptable salt, hydrate, or solvate thereof.

42. The pharmaceutical combination of any one of claims 1-41, wherein G is 4,5,6,7-tetrahydropyrazolo[1,5-a]pyridin-3-yl.

43. The pharmaceutical combination of any one of claims 1-41, wherein G is 1H-indol-3-yl.

44. The pharmaceutical combination of any one of claims 1-41, wherein G is -methyl-1-1H-indol-3-yl.

45. The pharmaceutical combination of any one of claims 1-41, wherein G is pyrazolo[1,5-a]pyridin-3-yl.

46. The pharmaceutical combination of any one of claims 1-45, wherein R_{O1} is H, F, Cl, or methyl.

47. The pharmaceutical combination of any one of claims 1-45, wherein R_{O1} is H.

48. The pharmaceutical combination of any one of claims 1-45, wherein R_{O1} is F or Cl.

49. The pharmaceutical combination of any one of claims 1-45, wherein R_{O1} is methyl.

50. The pharmaceutical combination of any one of claims 1-49, wherein R_{O2} is methoxy.

51. The pharmaceutical combination of any one of claims 1-49, wherein R_{O2} is methyl.

52. The pharmaceutical combination of any one of claims 1-51, wherein R_{O3} is (3R)-3-(dimethylamino)pyrrolidin-1-yl, (3S)-3-(dimethylamino)pyrrolidin-1-yl, 3-(dimethylamino)azetidin-1-yl, 5-methyl-2,5-diazaspiro[3.4]oct-2-yl, (3aR,6aR)-5-methylhexahydro-pyrrolo[3,4-b]pyrrol-1(2H)-yl, 1-methyl-1,2,3,6-tetrahydropyridin-4-yl, 4-methylpiperizin-1-yl, 4-(2-(dimethylamino)-2-oxoethyl)piperazin-1-yl, 1-amino-1,2,3,6-tetrahydropyridin-4-yl, or 4-((2S)-2-aminopropanoyl)piperazin-1-yl.

53. The pharmaceutical combination of any one of claims 1-51, wherein R_{O3} is (2-(dimethylamino)ethyl)-methylamino, (2-(methylamino)ethyl)-methylamino, methyl(2-(4-methylpiperazin-1-yl)ethyl)amino, or methyl(2-(morpholin-4-yl)ethyl)amino.

54. The pharmaceutical combination of any one of claims 1-51, wherein R_{O3} is (2-(dimethylamino)ethyl)-methylamino or (2-(methylamino)ethyl)-methylamino.

55. The pharmaceutical combination of any one of claims 1-41, wherein the ATP-competitive EGFR inhibitor is a compound of Formula I′a or I′b: 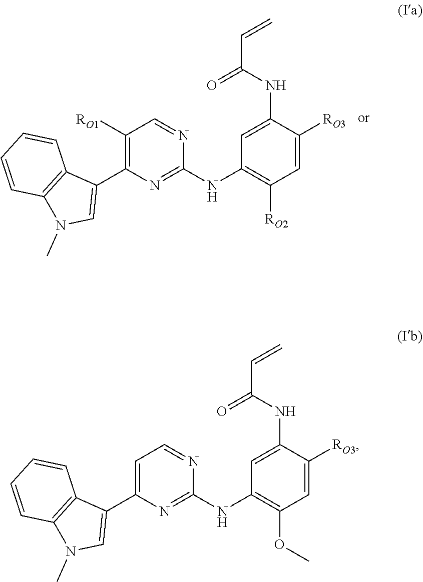
or a pharmaceutically acceptable salt, hydrate, or solvate thereof.

56. The pharmaceutical combination of any one of claims 1-41, wherein the ATP-competitive EGFR inhibitor is Compound O: 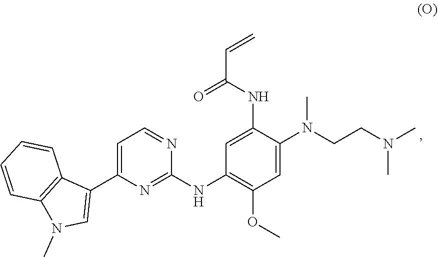 [SMILES: C=CC(=O)Nc1cc(Nc2nccc(-c3cn(C)c4ccccc34)n2)c(OC)cc1N(C)CCN(C)C]
or a pharmaceutically acceptable salt, hydrate, or solvate thereof.

57. A pharmaceutical combination comprising an allosteric EGFR inhibitor and an ATP-competitive EGFR inhibitor, wherein:
the allosteric EGFR inhibitor is Compound A: 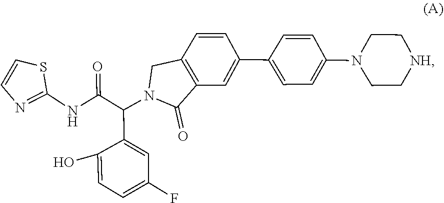 [SMILES: O=C(Nc1nccs1)C(c1cc(F)ccc1O)N1Cc2ccc(-c3ccc(N4CCNCC4)cc3)cc2C1=O]
or a pharmaceutically acceptable salt, hydrate, or solvate thereof,
the ATP-competitive EGFR inhibitor is Compound O: 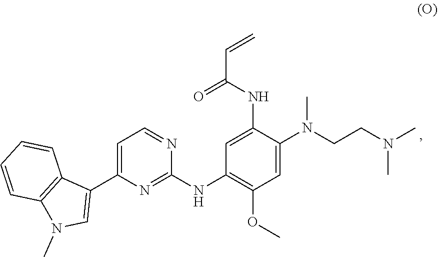 [SMILES: C=CC(=O)Nc1cc(Nc2nccc(-c3cn(C)c4ccccc34)n2)c(OC)cc1N(C)CCN(C)C]
or a pharmaceutically acceptable salt, hydrate, or solvate thereof,

58. A pharmaceutical composition comprising a pharmaceutical combination of any one of claims 1-57, and a pharmaceutically acceptable carrier, optionally further comprising a second agent that prevents EGFR dimer formation, and a pharmaceutically acceptable carrier.

59. A kit comprising an allosteric EGFR inhibitor of any one of claims 1-41 and 57 and an ATP-competitive EGFR inhibitor of any one of claims 1 and 42-57, optionally further comprising a second agent that prevents EGFR dimer formation.

60. A method of inhibiting a kinase, comprising administering to a subject in need thereof an effective amount of an allosteric EGFR inhibitor of any one of claims 1-41 and 57, in temporal proximity with an effective amount of an ATP-competitive EGFR inhibitor of any one of claims 1 and 42-57, or an effective amount of a pharmaceutical combination of any one of claims 1-57.

61. A method of treating or preventing a disease, a disease resistant to an EGFR targeted therapy, cancer wherein the cell of the cancer comprises an activated EGFR or an activated ERBB2, or cancer in a subject wherein the subject is identified as being in need of EGFR inhibition or ERBB2 inhibition for the treatment or prevention of cancer, comprising administering to a subject in need thereof an effective amount of an allosteric EGFR inhibitor of any one of claims 1-41 and 57, in temporal proximity with an effective amount of an ATP-competitive EGFR inhibitor of any one of claims 1 and 42-57, or an effective amount of a pharmaceutical combination of any one of claims 1-57.

62. The method of claim 61 or 62, further comprising administering a second agent that prevents EGFR dimer formation, and a pharmaceutically acceptable carrier.

63. An allosteric EGFR inhibitor according to any one of claims 1-41 and 57 for use in combination with an ATP-competitive EGFR inhibitor according to any one of claims 1 and 42-57, for
inhibiting a kinase in a subject in need thereof,
treating or preventing a disease in a subject in need thereof,
treating or preventing a disease resistant to an EGFR targeted therapy in a subject in need thereof,
treating or preventing cancer in a subject in need thereof, wherein the cell of the cancer comprises an activated EGFR or an activated ERBB2, or
treating or preventing cancer in a subject, wherein the subject is identified as being in need of EGFR inhibition or ERBB2 inhibition for the treatment or prevention of cancer.

64. Use of an allosteric EGFR inhibitor according to any one of claims 1-41 and 57 in combination with an ATP-competitive EGFR inhibitor according to any one of claims 1 and 42-57, for
inhibiting a kinase in a subject in need thereof,
treating or preventing a disease in a subject in need thereof,
treating or preventing a disease resistant to an EGFR targeted therapy in a subject in need thereof,
treating or preventing cancer in a subject in need thereof, wherein the cell of the cancer comprises an activated EGFR or an activated ERBB2, or
treating or preventing cancer in a subject, wherein the subject is identified as being in need of EGFR inhibition or ERBB2 inhibition for the treatment or prevention of cancer.

65. A combination of an allosteric EGFR inhibitor according to any one of claims 1-41 and 57 and an ATP-competitive EGFR inhibitor according to any one of claims 1 and 42-57, for
inhibiting a kinase in a subject in need thereof,
treating or preventing a disease in a subject in need thereof,
treating or preventing a disease resistant to an EGFR targeted therapy in a subject in need thereof,
treating or preventing cancer in a subject in need thereof, wherein the cell of the cancer comprises an activated EGFR or an activated ERBB2, or
treating or preventing cancer in a subject, wherein the subject is identified as being in need of EGFR inhibition or ERBB2 inhibition for the treatment or prevention of cancer.

66. Use of a combination of an allosteric EGFR inhibitor according to any one of claims 1-41 and 57 and an ATP-competitive EGFR inhibitor according to any one of claims 1 and 42-57, in
inhibiting a kinase in a subject in need thereof,
treating or preventing a disease in a subject in need thereof,
treating or preventing a disease resistant to an EGFR targeted therapy in a subject in need thereof,
treating or preventing cancer in a subject in need thereof, wherein the cell of the cancer comprises an activated EGFR or an activated ERBB2, or
treating or preventing cancer in a subject, wherein the subject is identified as being in need of EGFR inhibition or ERBB2 inhibition for the treatment or prevention of cancer.

67. A combination of an allosteric EGFR inhibitor according to any one of claims 1-41 and 57 and an ATP-competitive EGFR inhibitor according to any one of claims 1 and 42-57, for use in the manufacture of a medicament for
inhibiting a kinase in a subject in need thereof,
treating or preventing a disease in a subject in need thereof,
treating or preventing a disease resistant to an EGFR targeted therapy in a subject in need thereof,
treating or preventing cancer in a subject in need thereof, wherein the cell of the cancer comprises an activated EGFR or an activated ERBB2, or
treating or preventing cancer in a subject, wherein the subject is identified as being in need of EGFR inhibition or ERBB2 inhibition for the treatment or prevention of cancer.

68. Use of a combination of an allosteric EGFR inhibitor according to any one of claims 1-41 and 57 and an ATP-competitive EGFR inhibitor according to any one of claims 1 and 42-57, in the manufacture of a medicament for
inhibiting a kinase in a subject in need thereof,
treating or preventing a disease in a subject in need thereof,
treating or preventing a disease resistant to an EGFR targeted therapy in a subject in need thereof,
treating or preventing cancer in a subject in need thereof, wherein the cell of the cancer comprises an activated EGFR or an activated ERBB2, or
treating or preventing cancer in a subject, wherein the subject is identified as being in need of EGFR inhibition or ERBB2 inhibition for the treatment or prevention of cancer.

69. A pharmaceutical combination according to any one of claims 1-57 for
inhibiting a kinase in a subject in need thereof,
treating or preventing a disease in a subject in need thereof,
treating or preventing a disease resistant to an EGFR targeted therapy in a subject in need thereof,
treating or preventing cancer in a subject in need thereof, wherein the cell of the cancer comprises an activated EGFR or an activated ERBB2, or
treating or preventing cancer in a subject, wherein the subject is identified as being in need of EGFR inhibition or ERBB2 inhibition for the treatment or prevention of cancer.

70. Use of a pharmaceutical combination according to any one of claims 1-57 for
inhibiting a kinase in a subject in need thereof,
treating or preventing a disease in a subject in need thereof,
treating or preventing a disease resistant to an EGFR targeted therapy in a subject in need thereof,
treating or preventing cancer in a subject in need thereof, wherein the cell of the cancer comprises an activated EGFR or an activated ERBB2, or
treating or preventing cancer in a subject, wherein the subject is identified as being in need of EGFR inhibition or ERBB2 inhibition for the treatment or prevention of cancer.

71. A pharmaceutical combination according to any one of claims 1-57 for use in the manufacture of a medicament for
inhibiting a kinase in a subject in need thereof,
treating or preventing a disease in a subject in need thereof,
treating or preventing a disease resistant to an EGFR targeted therapy in a subject in need thereof,
treating or preventing cancer in a subject in need thereof, wherein the cell of the cancer comprises an activated EGFR or an activated ERBB2, or
treating or preventing cancer in a subject, wherein the subject is identified as being in need of EGFR inhibition or ERBB2 inhibition for the treatment or prevention of cancer.

72. Use of a pharmaceutical combination according to any one of claims 1-57 in the manufacture of a medicament for
inhibiting a kinase in a subject in need thereof,
treating or preventing a disease in a subject in need thereof,
treating or preventing a disease resistant to an EGFR targeted therapy in a subject in need thereof,
treating or preventing cancer in a subject in need thereof, wherein the cell of the cancer comprises an activated EGFR or an activated ERBB2, or
treating or preventing cancer in a subject, wherein the subject is identified as being in need of EGFR inhibition or ERBB2 inhibition for the treatment or prevention of cancer.
